(19) United States
(12) Patent Application Publication
(10) Pub. No.: US 2020/0122133 A1
(43) Pub. Date: Apr. 23, 2020

(54) HETEROGENEOUS CATALYSTS AND USES THEREOF

(71) Applicant: SCG CHEMICALS CO., LTD., Bangkok (TH)

(72) Inventors: Andrew St. John Weller, Oxford (GB); Frederick Mark Chadwick, Oxford (GB)

(21) Appl. No.: 16/463,592

(22) PCT Filed: Nov. 22, 2017
(86) PCT No.: PCT/GB2017/053514

§ 371 Date: May 23, 2019

(30) Foreign Application Priority Data
Nov. 24, 2016 (GB) 1619935.8
Sep. 7, 2017 (GB) 1714399.1

(51) Int. Cl. B01J 31/24; B01J 31/22; B01J 31/14; C07C 5/25; C07C 5/42; C07F 15/00

(52) U.S. Cl. CPC B01J 31/2409; B01J 31/2295; B01J 31/2291; B01J 31/146; B01J 31/143; C07C 5/2581; C07C 2531/22; C07F 15/0073; B01J 2531/822; B01J 2231/52; B01J 2231/766; B01J 2231/543; B01J 2231/32; C07C 5/42

(57) ABSTRACT

Catalytic processes employing rhodium complexes are disclosed, wherein the catalytic processes involve an initial step of activation of a C—H bond present within a hydrocarbon substrate. In contrast to prior art techniques, the catalytic processes of the invention can be conducted at low temperatures, and the catalytic compounds are themselves highly recyclable. Also disclosed are the rhodium complexes themselves and processes of making them.

### CROSS-REFERENCE TO RELATED APPLICATIONS

This application is a U.S. national stage filing, under 35 U.S.C. § 371(c), of International Application No. PCT/GB2017/053514, filed on Nov. 22, 2017, which claims priority to United Kingdom Application No. 1714399.1, filed on Sep. 7, 2017; and United Kingdom Application No. 1619935.8, filed on Nov. 24, 2016. The entire contents of each of the aforementioned applications are incorporated herein by reference.

### INTRODUCTION

The present invention relates to catalytic processes employing particular rhodium catalysts, as well as to the rhodium catalysts themselves. More specifically, the present invention relates to the alkene isomerisation, transfer dehydrogenation and dimerization catalytic processes employing the rhodium catalysts.

### BACKGROUND OF THE INVENTION

The transition-metal promoted isomerisation of alkenes is an atom efficient process that has many applications in industry and finechemicals synthesis;¹⁻³ such as the Shell Higher Olefin Process,⁴ olefin conversion technologies that produce propene from butene/ethene mixtures,⁵⁻⁸ and the isomerisation of functionalised alkenes.⁹ Homogenous processes are well-studied for a wide range of transition metal catalysts^{1, 9-11} and commonly, although by no means exclusively, use catalysts based upon later transition metals such as Co,¹² _{Ni,}^{13, 14} Ru,^{15, 16}Rh,¹⁷⁻¹⁹Ir,²⁰⁻²² which operate at relatively low temperatures (e.g. 120° C. or lower), sometimes at room temperature.^{18, 19, 23-25} Heterogeneous, or supported, systems are also wellestablished, but these often require higher temperatures to operate.^{26,27} Alkene isomerisation also plays a key role in alkane dehydrogenation,²⁸ and subsequent tandem upgrading processes such as metathesis²⁹ or dimerisation,^{30,31} where the kinetic product of dehydrogenation is a terminal alkene that can then undergo isomerization (Scheme 1).³²

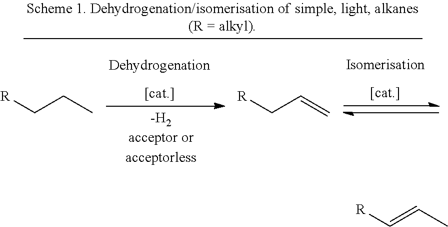

The dehydrogenation of light alkanes such as butane and pentane, and their subsequent isomerization is particularly interesting, as while these alkanes are unsuitable as transportation fuels or feedstock chemicals, their corresponding alkenes have myriad uses.^{30, 31, 33} The discovery of abundant sources of light alkanes in shale and offshore gas fields provides additional motivation to study their conversion into fuels and commodity chemicals.³⁴ As light alkanes are gaseous at, or close to, room temperature and pressure, the opportunity for solid/gas catalytic processes under these conditions is presented. Such conditions are also attractive due to physical separation of catalyst and substrates/product that they offer as well as opportunities to reduce catalyst decomposition through thermallyinduced processes.

Although heterogeneous solidgas systems for alkane dehydrogenation and alkene isomerization are well known,^{27, 35, 36} they often require high temperatures for their operation which leads to reductions in selectivity as well as catalyst deactivation through processes such a coking.

The present invention was devised with the foregoing in mind.

### SUMMARY OF THE INVENTION

According to a first aspect of the present invention there is provided a catalytic process comprising the step of:
a) activating one or more C—H bonds present within a C₄-C₁₀ hydrocarbon by contacting the C₄-C₁₀ hydrocarbon with a compound having a structure according to formula (I) shown below:

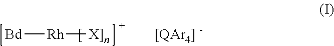

wherein
   Bd is a bidentate ligand bonded to Rh via two heteroatoms independently selected from P, N and S,
      wherein the two heteroatoms are independently optionally substituted with one or more substituents selected from iso-propyl, tert-butyl, sec-butyl, n-pentyl, iso-pentyl, neo-pentyl, tert-pentyl, sec-pentyl, 3-pentyl, iso-propoxy, tert-butoxy, sec-butoxy, n-pentoxy, iso-pentoxy, neo-pentoxy, tert-pentoxy, sec-pentoxy, 3-pentoxy, 6-8 membered carbocyclyl, 6-8 membered heterocyclyl, aryl or adamantyl, any of which may be optionally substituted with one or more substituents selected from halo, oxo, hydroxyl, (1-4C)alkyl, (2-4C)alkenyl, (2-4C)alkynyl, (1-4C)alkoxy, (1-4C)haloalkyl and N(Rₓ)(R_{y}),
         wherein Rₓ and R_{y} are each independently selected from hydrogen and (1-4C)alkyl;
   each X is independently a ligand that is weakly bound to Rh via one or more bond;
   n is 1 or 2;
   Q is selected from B, Al, In and Ga; and
   each Ar is independently
      i. a phenyl group substituted with one or more substituents selected from halo, (1-3C)alkyl and (1-3C)haloalkyl,
      ii. a (1-3C)alkoxy group substituted with one or more substituents selected from halo, (1-3C)alkyl and (1-3C)haloalkyl.

According to a further aspect of the present invention there is provided a catalytic process comprising the step of:
a) activating one or more C—H bonds present within a C₄-C₁₀ hydrocarbon by contacting the C₄-C₁₀ hydrocarbon with a compound having a structure according to formula (I) shown below:

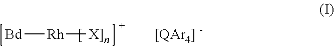

wherein
   Bd is a bidentate ligand bonded to Rh via two heteroatoms independently selected from P, N and S,
      wherein the two heteroatoms are independently optionally substituted with one or more substituents selected from iso-propyl, tert-butyl, sec-butyl, n-pentyl, iso-pentyl, neo-pentyl, tert-pentyl, sec-pentyl, 3-pentyl, iso-propoxy, tert-butoxy, sec-butoxy, n-pentoxy, iso-pentoxy, neo-pentoxy, tert-pentoxy, sec-pentoxy, 3-pentoxy, 6-8 membered carbocyclyl, 6-8 membered heterocyclyl, aryl or adamantyl, any of which may be optionally substituted with one or more substituents selected from halo, oxo, hydroxyl, (1-4C)alkyl, (2-4C)alkenyl, (2-4C)alkynyl, (1-4C)alkoxy, (1-4C)haloalkyl and N(Rₓ)(R_{y}),
      wherein Rₓ and R_{y} are each independently selected from hydrogen and (1-4C)alkyl;
   each X is independently a ligand that is weakly bound to Rh via one or more bond;
   n is 1 or 2;
   Q is selected from B, Al and In; and
   each Ar is independently a phenyl group substituted with one or more substituents selected from (1-3C)alkyl and (1-3C)haloalkyl.

According to a further aspect of the present invention there is provided a compound having a structure according to formula (Ia) shown below:

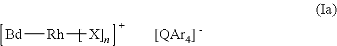

wherein
Bd is a bidentate ligand bonded to Rh via two heteroatoms independently selected from P, N and S,
   wherein the two heteroatoms are independently optionally substituted with one or more substituents selected from iso-propyl, tert-butyl, sec-butyl, n-pentyl, iso-pentyl, neo-pentyl, tert-pentyl, sec-pentyl, 3-pentyl, iso-propoxy, iso-butoxy, tert-butoxy, sec-butoxy, n-pentoxy, iso-pentoxy, neo-pentoxy, tert-pentoxy, sec-pentoxy, 3-pentoxy, 6-8 membered carbocyclyl, 6-8 membered heterocyclyl, aryl or adamantyl, any of which may be optionally substituted with one or more substituents selected from halo, oxo, hydroxyl, (1-4C)alkyl, (2-4C)alkenyl, (2-4C)alkynyl, (1-4C)alkoxy, (1-4C)haloalkyl and N(Rₓ)(R_{y}),
      wherein Rₓ and R_{y} are each independently selected from hydrogen and (1-4C)alkyl;
each X is independently a ligand that is weakly bound to Rh via one or more bond, wherein the total energy of coordination of Rh to each X is <130 KJmol⁻¹, and wherein each X is selected from hydrogen, dinitrogen, a linear or branched (2-10C)alkene, a 5-10 membered cycloalkene, a linear or branched (6-10C)alkane and a 8-10 membered cycloalkane, any of which may be optionally substituted with one or more substituents selected from halo, oxo, hydroxyl, (1-4C)alkyl, (2-4C)alkenyl, (2-4C)alkynyl, (1-4C)alkoxy, (1-4C)haloalkyl and —N(Rᵥ)(R_{w}),
   wherein Rᵥ and R_{w} are each independently selected from hydrogen and (1-4C)alkyl;
n is 1 or 2;
Q is selected from B, Al, In and Ga; and
each Ar is independently
   i. a phenyl group substituted with one or more substituents selected from halo, (1-3C)alkyl and (1-3C)haloalkyl, or
   ii. a (1-3C)alkoxy group substituted with one or more substituents selected from halo, (1-3C)alkyl and (1-3C)haloalkyl.

According to a further aspect of the present invention there is provided a compound having a structure according to formula (Ia) shown below:

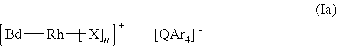

wherein
Bd is a bidentate ligand bonded to Rh via two heteroatoms independently selected from P, N and S,
   wherein the two heteroatoms are independently optionally substituted with one or more substituents selected from iso-propyl, tert-butyl, sec-butyl, n-pentyl, iso-pentyl, neo-pentyl, tert-pentyl, sec-pentyl, 3-pentyl, iso-propoxy, iso-butoxy, tert-butoxy, sec-butoxy, n-pentoxy, iso-pentoxy, neo-pentoxy, tert-pentoxy, sec-pentoxy, 3-pentoxy, 6-8 membered carbocyclyl, 6-8 membered heterocyclyl, aryl or adamantyl, any of which may be optionally substituted with one or more substituents selected from halo, oxo, hydroxyl, (1-4C)alkyl, (2-4C)alkenyl, (2-4C)alkynyl, (1-4C)alkoxy, (1-4C)haloalkyl and —N(Rₓ)(R_{y}),
      wherein Rₓ and R_{y} are each independently selected from hydrogen and (1-4C)alkyl;
each X is independently a ligand that is weakly bound to Rh via one or more bond, wherein the total energy of coordination of Rh to each X is <130 KJmol⁻¹, and wherein each X is selected from dinitrogen, a linear or branched (2-10C)alkene, a 5-10 membered cycloalkene, a linear or branched (6-10C)alkane and a 8-10 membered cycloalkane, any of which may be optionally substituted with one or more substituents selected from halo, oxo, hydroxyl, (1-4C)alkyl, (2-4C)alkenyl, (2-4C)alkynyl, (1-4C)alkoxy, (1-4C)haloalkyl and —N(Rᵥ)(R_{w}),
   wherein R, and R,, are each independently selected from hydrogen and (1-4C)alkyl;
n is 1 or 2;
Q is selected from B, Al and In; and
each Ar is independently a phenyl group substituted with one or more substituents selected from (1-3C)alkyl and (1-3C)haloalkyl.

### DETAILED DESCRIPTION OF THE INVENTION

### Definitions

Unless otherwise stated, the following terms used in the specification and claims have the following meanings set out below.

The term “(m-nC)” or “(m-nC) group” used alone or as a prefix, refers to any group having m to n carbon atoms.

The term “alkyl” includes both straight and branched chain alkyl groups. References to individual alkyl groups such as “propyl” are specific for the straight chain version only and references to individual branched chain alkyl groups such as “isopropyl” are specific for the branched chain version only. For example, “(1-6C)alkyl” includes (1-4C)alkyl, (1-3C)alkyl, propyl, isopropyl and t-butyl. A similar convention applies to other radicals, for example “phenyl(1-6C)alkyl” includes phenyl(1-4C)alkyl, benzyl, 1-phenylethyl and 2-phenylethyl.

The term “halo” refers to fluoro, chloro, bromo and iodo.

The term “haloalkyl” or “haloalkoxy” is used herein to refer to an alkyl or alkoxy group respectively in which one or more hydrogen atoms have been replaced by halogen (e.g. fluorine) atoms. Examples of haloalkyl and haloalkoxy groups include fluoroalkyl and fluoroalkoxy groups such as —CHF₂, —CH₂CF₃, or perfluoroalkyl/alkoxy groups such as —CF₃, —CF₂CF₃, —OCF₃, —OC(CF₃)₃ and —OCF₂CF₃.

The term “carbocyclyl”, “carbocyclic” or “carbocycle” means a non-aromatic, saturated or partially saturated monocyclic, or a fused, bridged, or spiro bicyclic ring system(s) based exclusively on carbon. Monocyclic carbocyclic rings contain from about 3 to 12 (suitably from 3 to 7) ring atoms. Bicyclic carbocycles contain from 7 to 17 carbon atoms in the rings, suitably 7 to 12 carbon atoms, in the rings. Bicyclic carbocyclic rings may be fused, spiro, or bridged ring systems.

The term “cycloalkyl” or “cycloalkane” means a saturated fused, bridged, or spiro bicyclic ring system(s) based exclusively on carbon. Monocyclic cycloalkanes contain from about 3 to 12 (suitably from 3 to 7) ring atoms. Bicyclic cycloalkanes contain from 7 to 17 carbon atoms in the rings, suitably 7 to 12 carbon atoms, in the rings. Bicyclic cycloalkanes may be fused, spiro, or bridged ring systems.

The term “cycloalkenyl” or “cycloalkene” means an unsaturated fused, bridged, or spiro bicyclic ring system(s) based exclusively on carbon. Monocyclic cycloalkenes contain from about 6 to 12 (suitably from 6 to 7) ring atoms. Bicyclic cycloalkenes contain from 7 to 17 carbon atoms in the rings, suitably 7 to 12 carbon atoms, in the rings. Bicyclic cycloalkenes may be fused, spiro, or bridged ring systems.

The term “heterocyclyl”, “heterocyclic” or “heterocycle” means a non-aromatic saturated or partially saturated monocyclic, fused, bridged, or spiro bicyclic heterocyclic ring system(s). Monocyclic heterocyclic rings contain from about 3 to 12 (suitably from 3 to 7) ring atoms, with from 1 to 5 (suitably 1, 2 or 3) heteroatoms selected from nitrogen, oxygen or sulfur in the ring. Bicyclic heterocycles contain from 7 to 17 member atoms, suitably 7 to 12 member atoms, in the ring. Bicyclic heterocyclic(s) rings may be fused, spiro, or bridged ring systems. Examples of heterocyclic groups include cyclic ethers such as oxiranyl, oxetanyl, tetrahydrofuranyl, dioxanyl, and substituted cyclic ethers. Heterocycles containing nitrogen include, for example, azetidinyl, pyrrolidinyl, piperidinyl, piperazinyl, tetrahydrotriazinyl, tetrahydropyrazolyl, and the like. Typical sulfur containing heterocycles include tetrahydrothienyl, dihydro-1,3-dithiol, tetrahydro-2H-thiopyran, and hexahydrothiepine. Other heterocycles include dihydro-oxathiolyl, tetrahydro-oxazolyl, tetrahydro-oxadiazolyl, tetrahydrodioxazolyl, tetrahydro-oxathiazolyl, hexahydrotriazinyl, tetrahydro-oxazinyl, morpholinyl, thiomorpholinyl, tetrahydropyrimidinyl, dioxolinyl, octahydrobenzofuranyl, octahydrobenzimidazolyl, and octahydrobenzothiazolyl. For heterocycles containing sulfur, the oxidized sulfur heterocycles containing SO or SO₂ groups are also included. Examples include the sulfoxide and sulfone forms of tetrahydrothienyl and thiomorpholinyl such as tetrahydrothiene 1,1-dioxide and thiomorpholinyl 1,1-dioxide. A suitable value for a heterocyclyl group which bears 1 or 2 oxo (═O) or thioxo (═S) substituents is, for example, 2-oxopyrrolidinyl, 2-thioxopyrrolidinyl, 2-oxoimidazolidinyl, 2-thioxoimidazolidinyl, 2-oxopiperidinyl, 2,5-dioxopyrrolidinyl, 2,5-dioxoimidazolidinyl or 2,6-dioxopiperidinyl. Particular heterocyclyl groups are saturated monocyclic 3 to 7 membered heterocyclyls containing 1, 2 or 3 heteroatoms selected from nitrogen, oxygen or sulfur, for example azetidinyl, tetrahydrofuranyl, tetrahydropyranyl, pyrrolidinyl, morpholinyl, tetrahydrothienyl, tetrahydrothienyl 1,1-dioxide, thiomorpholinyl, thiomorpholinyl 1,1-dioxide, piperidinyl, homopiperidinyl, piperazinyl or homopiperazinyl. As the skilled person would appreciate, any heterocycle may be linked to another group via any suitable atom, such as via a carbon or nitrogen atom. Suitably, the term “heterocyclyl”, “heterocyclic” or “heterocycle” will refer to 4, 5, 6 or 7 membered monocyclic rings as defined above. In a particular embodiment, heterocyclyl is tetrahydropyranyl.

The term “heteroaryl” or “heteroaromatic” means an aromatic mono-, bi-, or polycyclic ring incorporating one or more (for example 1-4, particularly 1, 2 or 3) heteroatoms selected from nitrogen, oxygen or sulfur. Examples of heteroaryl groups are monocyclic and bicyclic groups containing from five to twelve ring members, and more usually from five to ten ring members. The heteroaryl group can be, for example, a 5- or 6-membered monocyclic ring or a 9- or 10-membered bicyclic ring, for example a bicyclic structure formed from fused five and six membered rings or two fused six membered rings. Each ring may contain up to about four heteroatoms typically selected from nitrogen, sulfur and oxygen. Typically the heteroaryl ring will contain up to 3 heteroatoms, more usually up to 2, for example a single heteroatom. In one embodiment, the heteroaryl ring contains at least one ring nitrogen atom. The nitrogen atoms in the heteroaryl rings can be basic, as in the case of an imidazole or pyridine, or essentially non-basic as in the case of an indole or pyrrole nitrogen. In general the number of basic nitrogen atoms present in the heteroaryl group, including any amino group substituents of the ring, will be less than five. Suitably, the term “heteroaryl” or “heteroaromatic” will refer to 5 or 6 membered monocyclic hetyeroaryl rings as defined above.

The term “aryl” means a cyclic or polycyclic aromatic ring having from 5 to 12 carbon atoms. The term aryl includes both monovalent species and divalent species. Examples of aryl groups include, but are not limited to, phenyl, biphenyl, naphthyl and the like. Typically, aryl is phenyl.

The term “optionally substituted” refers to either groups, structures, or molecules that are substituted and those that are not substituted. It will be understood that substitutions may only occur at sites where it is chemically feasible to do so.

Where optional substituents are chosen from “one or more” groups it is to be understood that this definition includes all substituents being chosen from one of the specified groups or the substituents being chosen from two or more of the specified groups.

Catalytic processes of the invention

As described hereinbefore, the present invention provides a catalytic process comprising the step of:
a) activating one or more C—H bonds present within a C₄-C₁₀ hydrocarbon by contacting the C₄-C₁₀ hydrocarbon with a compound having a structure according to formula (I) shown below:

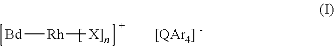

wherein
   Bd is a bidentate ligand bonded to Rh via two heteroatoms independently selected from P, N and S,
      wherein the two heteroatoms are independently optionally substituted with one or more substituents selected from iso-propyl, tert-butyl, sec-butyl, n-pentyl, iso-pentyl, neo-pentyl, tert-pentyl, sec-pentyl, 3-pentyl, iso-propoxy, tert-butoxy, sec-butoxy, n-pentoxy, iso-pentoxy, neo-pentoxy, tert-pentoxy, sec-pentoxy, 3-pentoxy, 6-8 membered carbocyclyl, 6-8 membered heterocyclyl, aryl or adamantyl, any of which may be optionally substituted with one or more substituents selected from halo, oxo, hydroxyl, (1-4C)alkyl, (2-4C)alkenyl, (2-4C)alkynyl, (1-4C)alkoxy, (1-4C)haloalkyl and —N(Rₓ)(R_{y}),
      wherein Rₓ and R_{y} are each independently selected from hydrogen and (1-4C)alkyl;
   each X is independently a ligand that is weakly bound to Rh via one or more bond;
   n is 1, 2 or 3;
   Q is selected from B, Al, In and Ga; and
   each Ar is independently
   i. a phenyl group substituted with one or more substituents selected from halo, (1-3C)alkyl and (1-3C)haloalkyl, or
      ii. a (1-3C)alkoxy group substituted with one or more substituents selected from halo, (1-3C)alkyl and (1-3C)haloalkyl.

The numerous benefits of heterogeneous catalytic systems (wherein the catalyst is provided in the solid state, with the reagent being provided in a liquid or gaseous state) are well documented. As alluded to hereinbefore, although various heterogeneous solidgas catalytic systems for catalytic processes involving C—H bond activation (e.g. alkane dehydrogenation and alkene isomerization) are known,^{27, 35, 36} they often require high temperatures for their operation. Industrially, this is sub-optimal for a variety of reasons. Not only does the requirement for high temperatures have environmental consequences, but the elevated temperatures can themselves hamper catalytic performance (e.g. by loss of selectivity), as well as shorten the lifetime of the catalyst by thermally-induced decomposition (e.g. by coking). Hence, the poor recyclability of such catalysts, coupled to the high temperatures required for their operation, can result in high operating costs on an industrial scale.

When compared with prior art C—H bond activation catalytic processes, the catalytic processes of the invention offer a number of advantages. Chiefly, the solid-phase compounds of formula (I) have been demonstrated to be catalytically active in catalytic processes involving C—H bond activation at temperatures significantly lower than currently available techniques. In particular, the compounds of formula (I) have been shown to exhibit significant catalytic activity in alkene isomerisation, alkane transfer dehydrogenation, and alkene dimerization reactions at room temperature. Moreover, the compounds of formula (I) exhibit remarkable long-term stability, as well as notable catalytic recyclability.

In an embodiment,
Bd is a bidentate ligand bonded to Rh via two heteroatoms independently selected from P, N and S, wherein the two heteroatoms are independently optionally substituted with one or more substituents selected from iso-propyl, tert-butyl, sec-butyl, n-pentyl, iso-pentyl, neo-pentyl, tea-pentyl, sec-pentyl, 3-pentyl, iso-propoxy, tert-butoxy, sec-butoxy, n-pentoxy, iso-pentoxy, neo-pentoxy, tert-pentoxy, sec-pentoxy, 3-pentoxy, 6-8 membered carbocyclyl, 6-8 membered heterocyclyl, aryl or adamantyl, any of which may be optionally substituted with one or more substituents selected from halo, oxo, hydroxyl, (1-4C)alkyl, (2-4C)alkenyl, (2-4C)alkynyl, (1-4C)alkoxy, (1-4C)haloalkyl and —N(Rₓ)(R_{y}),

wherein Rₓ and R_{y} are each independently selected from hydrogen and (1-4C)alkyl;
each X is independently a ligand that is weakly bound to Rh via one or more bond;
n is 1, 2 or 3;
Q is selected from B, Al and In; and
each Ar is independently a phenyl group substituted with one or more substituents selected from (1-3C)alkyl and (1-3C)haloalkyl.

In an embodiment, the two heteroatoms of Bd are independently substituted with one or more substituents selected from iso-propyl, tert-butyl, sec-butyl, iso-propoxy, tert-butoxy, sec-butoxy, 6-8 membered carbocyclyl, 6-8 membered heterocyclyl (e.g. tetrahydropyranyl), aryl or adamantyl, any of which may be optionally substituted with one or more substituents selected from halo, hydroxyl, (1-4C)alkyl, (1-4C)alkoxy, and —N(Rₓ)(R_{y}),

wherein Rₓ and R_{y} are each independently selected from hydrogen and (1-4C)alkyl.

In an embodiment, the two heteroatoms of Bd are independently substituted with one or more substituents selected from iso-propyl, tert-butyl, 6-8 membered carbocyclyl, aryl or adamantyl, any of which may be optionally substituted with one or more substituents selected from halo, hydroxyl, (1-4C)alkyl and (1-4C)alkoxy.

In an embodiment, the two heteroatoms of Bd are independently substituted with one or more substituents selected from iso-propyl, 6-8 membered carbocyclyl, aryl or adamantyl, any of which may be optionally substituted with one or more substituents selected from halo, hydroxyl, (1-4C)alkyl and (1-4C)alkoxy.

In an embodiment, the two heteroatoms of Bd are independently substituted with one or more substituents selected from iso-propyl, cyclohexyl or aryl, any of which may be optionally substituted with one or more substituents selected from halo, hydroxyl, (1-4C)alkyl and (1-4C)alkoxy.

In an embodiment, the two heteroatoms of Bd are independently substituted with one or more cyclohexyl substituents, any of which may be substituted with one or more substituents selected from halo, hydroxyl, (1-4C)alkyl and (1-4C)alkoxy.

In an embodiment, the two heteroatoms of Bd are each substituted with two cyclohexyl substituents.

In an embodiment, Bd is a bis-phosphine or bis-amine bidentate ligand.

In an embodiment, Bd is a bis-amine bidentate ligand.

In an embodiment, Bd is a bis-amine bidentate ligand selected from ethylenediamine, 1,4-diazadiene, 1,1′-bipyridine, 1,10-phenanthroline and ethylenediaminetetraacetate, wherein one or more of the N atoms is independently optionally substituted with one or more substituents as defined hereinbefore in respect of Bd.

In an embodiment, Bd is a bis-phosphine bidentate ligand. The bis-phosphine bidentate ligand may have a structure according to formula (II) shown below:

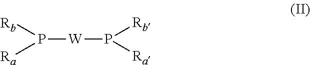

wherein

Rₐ, Rₐ′, R_{b} and R_{b}′ are each independently iso-propyl, tert-butyl, sec-butyl, n-pentyl, iso-pentyl, neo-pentyl, tert-pentyl, sec-pentyl, 3-pentyl, iso-propoxy, tert-butoxy, sec-butoxy, n-pentoxy, iso-pentoxy, neo-pentoxy, tert-pentoxy, sec-pentoxy, 3-pentoxy, 6-8 membered carbocyclyl, 6-8 membered heterocyclyl, aryl or adamantyl, any of which may be optionally substituted with one or more substituents selected from halo, oxo, hydroxyl, (1-4C)alkyl, (2-4C)alkenyl, (2-4C)alkynyl, (1-4C)alkoxy, (1-4C)haloalkyl and —N(Rₓ)(R_{y}),
wherein Rₓ and R_{y} are each independently selected from hydrogen and (1-4C)alkyl; and

W is a (1-5C)alkylene linking group, wherein one or more of the carbon atoms may be replaced with a heteroatom selected from N, O and S, and wherein W is optionally substituted with one or more groups R_{c}, wherein each R_{c} is independently selected from halo, oxo, hydroxyl, (1-4C)alkyl, (2-4C)alkenyl, (2-4C)alkynyl, (1-4C)alkoxy, (1-4C)haloalkyl and aryl,
and/or two groups R_{c} may be linked, such that when taken with the atoms to which they are attached, they form a phenyl group optionally fused to a phenyl or 5-6-membered heteroaryl, wherein any or all of the rings are optionally substituted with one or more substituents selected from halo and (1-4C)alkyl.

In an embodiment, the bis-phosphine bidentate ligand has a structure according to formula (II), wherein W is a (1-5C)alkylene linking group optionally substituted with one or more groups R_{c}, wherein each R_{c} is independently (1-4C)alkyl or (1-4C)alkoxy, and/or two groups R_{c} may be linked, such that when taken with the atoms to which they are attached, they form a phenyl group optionally substituted with one or more substituents selected from halo, (1-4C)alkyl and (1-4C)alkoxy.

In an embodiment, the bis-phosphine bidentate ligand has a structure according to formula (II), wherein W is a (1-5C)alkylene linking group optionally substituted with one or more groups R_{c}, wherein each R_{c} is independently (1-4C)alkyl, and/or two groups R_{c} may be linked, such that when taken with the atoms to which they are attached, they form a phenyl group optionally substituted with one or more substituents selected from halo and (1-4C)alkyl.

In an embodiment, the bis-phosphine bidentate ligand has a structure according to formula (II), wherein W has any of the following structures:

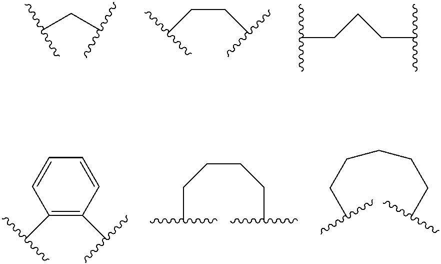

In an embodiment, the bis-phosphine bidentate ligand has a structure according to formula (II), wherein W has any of the following structures:

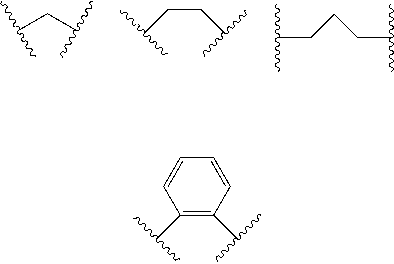

In an embodiment, the bis-phosphine bidentate ligand has a structure according to formula (II), wherein W is ethylene, propylene, butylene or pentylene.

In an embodiment, the bis-phosphine bidentate ligand has a structure according to formula (II), wherein W is ethylene.

In an embodiment, the bis-phosphine bidentate ligand has a structure according to formula (II), wherein Rₐ, Rₐ′, R_{b} and R_{b}′ are each independently iso-propyl, tert-butyl, sec-butyl, iso-propoxy, tert-butoxy, sec-butoxy, 6-8 membered carbocyclyl, 6-8 membered heterocyclyl (e.g. tetrahydropyranyl), aryl or adamantyl, any of which may be optionally substituted with one or more substituents selected from halo, hydroxyl, (1-4C)alkyl, (1-4C)alkoxy, and N(Rₓ)(R_{y}), wherein Rₓ and R_{y} are each independently selected from hydrogen and (1-4C)alkyl.

In an embodiment, the bis-phosphine bidentate ligand has a structure according to formula (II), wherein Rₐ, Rₐ′, R_{b} and R_{b}′ are each independently iso-propyl, tert-butyl, 6-8 membered carbocyclyl, aryl or adamantyl, any of which may be optionally substituted with one or more substituents selected from halo, hydroxyl, (1-4C)alkyl and (1-4C)alkoxy.

In an embodiment, the bis-phosphine bidentate ligand has a structure according to formula (II), wherein Rₐ, Rₐ′, R_{b} and R_{b}′ are each independently iso-propyl, 6-8 membered carbocyclyl, aryl or adamantyl, any of which may be optionally substituted with one or more substituents selected from halo, hydroxyl, (1-4C)alkyl and (1-4C)alkoxy.

In an embodiment, the bis-phosphine bidentate ligand has a structure according to formula (II), wherein Rₐ, Rₐ′, R_{b} and R_{b}′ are each independently iso-propyl, cyclohexyl or aryl, any of which may be optionally substituted with one or more substituents selected from halo, hydroxyl, (1-4C)alkyl and (1-4C)alkoxy.

In an embodiment, the bis-phosphine bidentate ligand has a structure according to formula (II), wherein Rₐ, Rₐ′, R_{b} and R_{b}′ are cyclohexyl, any of which may be substituted with one or more substituents selected from halo, hydroxyl, (1-4C)alkyl and (1-4C)alkoxy.

In an embodiment, the bis-phosphine bidentate ligand has a structure according to formula (II), wherein Rₐ, Rₐ′, R_{b} and R_{b}′ are cyclohexyl.

Each X is a weakly bound ligand. It will be appreciated by those of skill in the art that the strength of binding between Rh and X has important implications for the catalytic processes of the invention. In particular, it will be appreciated that a weakly bound ligand X is one that can be displaced by the C₄-C₁₀ hydrocarbon during step a) of the catalytic process. In an embodiment, each X is independently a ligand weakly bound to Rh via one or more bond, wherein the total energy of coordination of Rh to each X is <130 KJ mol⁻¹. Suitably, each X is independently a ligand weakly bound to Rh via one or more bond, wherein the total energy of coordination of Rh to each X is 5-130 KJ mol⁻¹. More suitably, each X is independently a ligand weakly bound to Rh via one or more bond, wherein the total energy of coordination of Rh to each X is 5-125 KJ mol⁻¹. Yet more suitably, each X is independently a ligand weakly bound to Rh via one or more bond, wherein the total energy of coordination of Rh to each X is 5-122 KJ mol⁻¹. Most suitably, each X is independently a ligand weakly bound to Rh via one or more bond, wherein the total energy of coordination of Rh to each X is 5-118 KJ mol⁻¹.

In an embodiment, each X is hydrogen, an alkane, an alkene or dinitrogen

In an embodiment, each X is an alkane, an alkene or dinitrogen.

In an embodiment, each X is selected from hydrogen, dinitrogen, a linear or branched (2-10C)alkene, a 5-10 membered cycloalkene, a linear or branched (2-10C)alkane and a 5-10 membered cycloalkane, any of which may be optionally substituted with one or more substituents selected from halo, oxo, hydroxyl, (1-4C)alkyl, (2-4C)alkenyl, (2-4C)alkynyl, (1-4C)alkoxy, (1-4C)haloalkyl and —N(Rₓ)(R_{y}),

wherein Rₓ and R_{y} are each independently selected from hydrogen and (1-4C)alkyl.

In an embodiment, each X is selected from hydrogen, dinitrogen, a linear or branched (2-10C)alkene, a monounsaturated 5-10 membered cycloalkene, a branched (2-10C)alkane and a 5-10 membered cycloalkane, any of which may be optionally substituted with one or more substituents selected from halo, oxo, hydroxyl, (1-4C)alkyl, (2-4C)alkenyl, (2-4C)alkynyl, (1-4C)alkoxy and (1-4C)haloalkyl.

In an embodiment, each X is selected from hydrogen, dinitrogen, a linear or branched (2-8C)alkene, a monounsaturated 5-8 membered cycloalkene, a branched (6-10C)alkane and a 5-10 membered cycloalkane, any of which may be optionally substituted with one or more substituents selected from halo, oxo, hydroxyl, (1-4C)alkyl, (2-4C)alkenyl, (2-4C)alkynyl, (1-4C)alkoxy and (1-4C)haloalkyl.

In an embodiment, each X is selected from dinitrogen, a linear or branched (2-10C)alkene, a 5-10 membered cycloalkene, a linear or branched (2-10C)alkane and a 5-10 membered cycloalkane, any of which may be optionally substituted with one or more substituents selected from halo, oxo, hydroxyl, (1-4C)alkyl, (2-4C)alkenyl, (2-4C)alkynyl, (1-4C)alkoxy, (1-4C)haloalkyl and —N(Rₓ)(R_{y}),

wherein Rₓ and R_{y} are each independently selected from hydrogen and (1-4C)alkyl.

In an embodiment, each X is selected from dinitrogen, a linear or branched (2-10C)alkene, a monounsaturated 5-10 membered cycloalkene, a branched (2-10C)alkane and a 5-10 membered cycloalkane, any of which may be optionally substituted with one or more substituents selected from halo, oxo, hydroxyl, (1-4C)alkyl, (2-4C)alkenyl, (2-4C)alkynyl, (1-4C)alkoxy and (1-4C)haloalkyl.

In an embodiment, each X is selected from dinitrogen, a linear or branched (2-8C)alkene, a monounsaturated 5-8 membered cycloalkene, a branched (6-10C)alkane and a 5-10 membered cycloalkane, any of which may be optionally substituted with one or more substituents selected from halo, oxo, hydroxyl, (1-4C)alkyl, (2-4C)alkenyl, (2-4C)alkynyl, (1-4C)alkoxy and (1-4C)haloalkyl.

Exemplary linear alkenes include ethene, propene, butene and hexene.

Exemplary 5-10 membered cycloalkenes include cycloheptene and cyclooctene. Other exemplary 5-10 membered cycloalkenes include norbornene.

Exemplary 5-10 membered cycloalkanes are illustrated below:

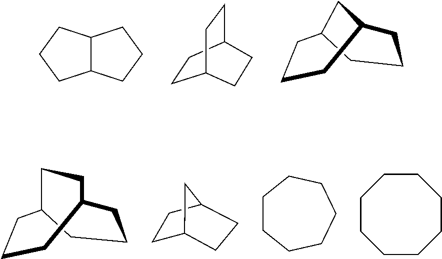

In an embodiment, each X is selected from hydrogen, ethene, propene, butene, hexane, cyclooctene and norbornane. Suitably, each X is ethene or norbornane.

In an embodiment, each X is selected from ethene, propene, butene, hexene and norbornane. Suitably, each X is ethene or norbornane.

It will be understood that the nature of bonding between Rh and X will depend on the nature of X. When X is ethene, each ethene ligand may be η² coordinated to Rh. When X is norbornane, the norbornane ligand is coordinated to Rh by a 3-centre 2-electron sigma interaction between the C—H bond of the norbornane and the metal centre.

It will be understood that the value of n depends on the nature of X. For smaller X ligands (e.g. ethene or hydrogen), Rh can accommodate two or three X ligands (e.g. n=2 or 3). For larger X ligands (e.g. norbornane), Rh can accommodate only one X ligand (e.g. n=1). Suitably, n is 1 or 2.

In an embodiment, Q is boron or aluminium.

In an embodiment, Q is boron.

In an embodiment, each Ar is either i) a phenyl group substituted at the 3-, 4- and/or 5-position with one or more substituents selected from halo (1-3C)alkyl and (1-3C)haloalkyl, or ii) a (1-3C)alkoxy group substituted with one or more substituents selected from halo (1-3C)alkyl and (1-3C) haloalkyl.

In an embodiment, each Ar is either i) a phenyl group substituted at the 3- and/or 5-position with one or more substituents selected from fluoro, chloro, (1-3C)alkyl and (1-3C)haloalkyl, or ii) a (1-3C)alkoxy group substituted with one or more substituents selected from fluoro, chloro and (1-2C)haloalkyl.

In an embodiment, each Ar is either i) a phenyl group substituted at the 3- and/or 5-position with one or more substituents selected from fluoro, chloro, (1-2C)alkyl and (1-2C)fluoroalkyl, or ii) a (1-2C)alkoxy group substituted with one or more substituents selected from fluoro, chloro and (1-2C)haloalkyl.

In an embodiment, each Ar is a phenyl group substituted at the 3-, 4- and/or 5-position with one or more substituents selected from (1-3C)alkyl and (1-3C)haloalkyl.

In an embodiment, each Ar is a phenyl group substituted at the 3- and/or 5-position with one or more substituents selected from (1-3C)alkyl and (1-3C)haloalkyl.

In an embodiment, each Ar is a phenyl group substituted at the 3- and/or 5-position with one or more substituents selected from (1-2C)alkyl and (1-2C)fluoroalkyl.

In an embodiment, each Ar is a phenyl group substituted at both the 3- and 5-position with a substituent selected from (1-2C)alkyl and (1-2C)fluoroalkyl.

In an embodiment, each Ar is a phenyl group substituted at both the 3- and 5-position with trifluoromethyl.

In an embodiment, [QAr₄] has any of the following structures:

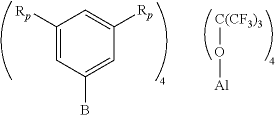

wherein Rₚ is fluoro, chloro, difluoromethyl or trifluromethyl. Suitably, Rₚ is fluoro, chloro or trifluromethyl.

In a particular embodiment, the compound of formula (I) has any of the following structures:

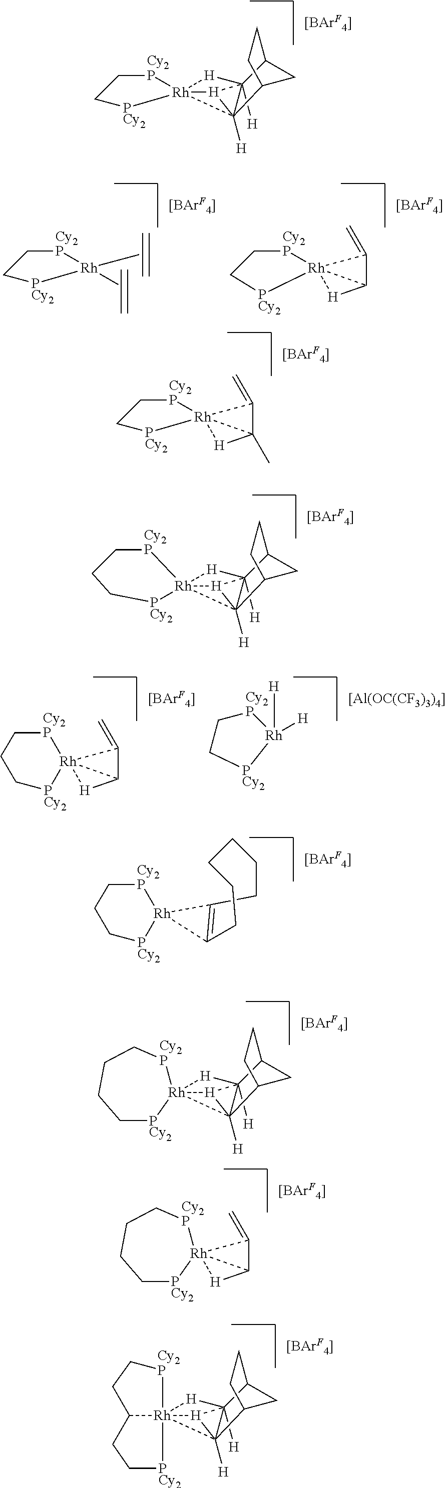

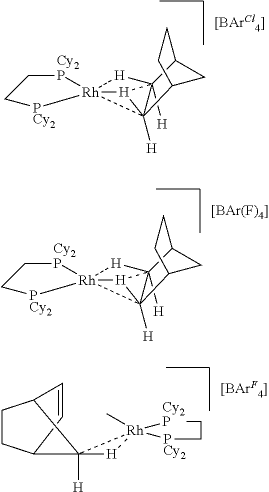

wherein ‘Cy’ denotes cyclohexyl,
‘Ar^{F}’ denotes 3,5-(CF₃)₂C₆H₃,
‘Ar^{a}’ denotes 3,5-(CI)₂C₆H₃, and
‘Ar(F)’ denotes 3,5-(F)₂C₆H₃.

In a particular embodiment, the compound of formula (I) has any of the following structures:

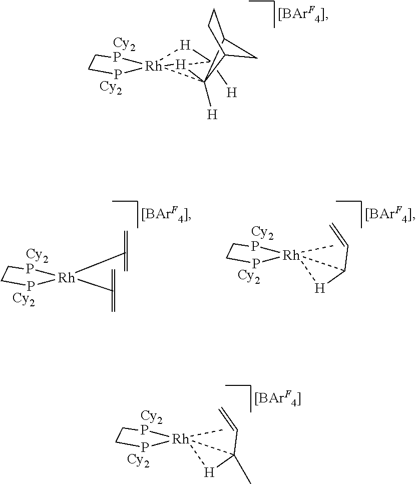

wherein ‘Cy’ denotes cyclohexyl and ‘Ar^{F}’ denotes 3,5-(CF₃)₂C₆H₃.

In a particular embodiment, the compound of formula (I) has either of the following structures:

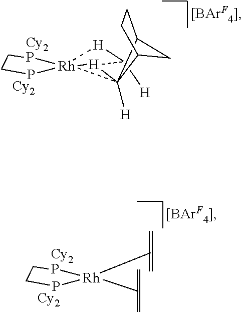

wherein ‘Cy’ denotes cyclohexyl and ‘Ar^{F}’ denotes 3,5-(CF₃)₂C₆H₃.

In an embodiment, the compound of formula (I) is a solid. Suitably, the compound of formula (I) is crystalline.

In an embodiment, the compound of formula (I) is unsupported. By virtue of their crystalline morphology, the compounds of formula (I) are themselves suitable for direct use in heterogeneous catalytic systems, without the need for being supported on a separate solid support (e.g. silica or alumina).

In an embodiment, the compound of formula (I) is

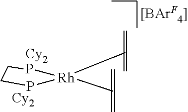

wherein ‘Cy’ denotes cyclohexyl and ‘Ar^{F}’ denotes 3,5-(CF₃)₂C₆H₃, and wherein the compounds has octahedral crystal morphology. The space groups is 02/c (No. 15 International Tables). Suitably, the X-ray powder diffraction pattern for the compound exhibits strong peaks at 2theta=9.1953 and 19.1186°.

In an embodiment, the compound of formula (I) is

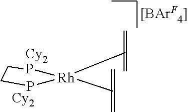

wherein ‘Cy’ denotes cyclohexyl and ‘Ar^{F}’ denotes 3,5-(CF₃)₂C₆H₃, and wherein the compounds has hexagonal crystal morphology. The space groups is P6₃22 (No. 182 International Tables). Suitably, the X-ray powder diffraction pattern for the compound exhibits strong peaks at 2theta=3.9514 and 6.8133°.

In an embodiment, the C₄-C₁₀ hydrocarbon is in the form of a liquid or gas, and the catalytic process is conducted in the heterogeneous state (the compound of formula (I) being a solid).

The concept of C—H bond activation will be readily understood by one of ordinary skill in the art. In particular, it will be appreciated that C—H bond activation refers to the cleavage of unreacted C—H bonds in hydrocarbons by transition metal complexes to form products containing one or more M-C bond (when M is the transition metal). A variety of catalytic processes employing transition metal-containing catalysts proceed via an initial step of activation of a C-H bond within a hydrocarbon substrate. Such catalytic processes include, but are not limited to, alkene isomerisation, alkane transfer dehydrogenation and alkene dimerization.

In an embodiment, the C₄-C₁₀ hydrocarbon is an alkene comprising one or more C═C bonds, and step a) results in the migration of the one or more C═C bonds within the alkene. In such embodiments, the catalytic process is an alkene isomerisation process.

It will be appreciated that within the alkene isomerisation process, the alkene may contain one or more double bonds. When the alkene contains more than one double bond, step a) may result in the migration of one or more double bonds. It will also be understood that the alkene may be linear or branched, and may be substituted with one or more substituents selected from halo, oxo, hydroxyl and amino. Suitably, the alkene is a terminal alkene (e.g. 1-butene) or an internal alkene (e.g. 2-butene). Depending on the nature of the 0₄-C₁₀ hydrocarbon, step a) may result in the formation of a terminal alkene, an internal alkene, or a mixture of both.

In an embodiment, the C₄-C₁₀ hydrocarbon is an alkene comprising one or more C═C bonds, and step a) results in the migration of the one or more C═C bonds within the alkene, wherein the alkene is a C₄-C₈ alkene.

In an embodiment, the C₄-C₁₀ hydrocarbon is an alkene comprising one or more C═C bonds, and step a) results in the migration of the one or more C═C bonds within the alkene, wherein the alkene is selected from 1-butene and 2-butene.

In an embodiment, the C₄-C₁₀ hydrocarbon is 1-butene and the process results in the conversion of the 1-butene to 2-butene. The process results in a mixture of cis and trans 2-butene isomers.

In an embodiment, the catalytic process is an alkene isomerisation process and step a) is conducted at a temperature of 0-100° C. Suitably, step a) is conducted at a temperature of 0-50° C. More suitably, step a) is conducted at a temperature of 0-30° C. Most suitably, step a) is conducted at a temperature of 18-30° C.

In an embodiment, the catalytic process is an alkene isomerisation process, wherein the molar ratio of the compound of formula (I) to the C₄-C₁₀ hydrocarbon in step a) is 1:1 to 1:100000. Suitably, the molar ratio of the compound of formula (I) to the C₄-C₁₀ hydrocarbon in step a) is 1:40 to 1:1000.

In another embodiment, the C₄-C₁₀ hydrocarbon is an alkane, and step a) is conducted in the presence of a hydrogen acceptor, and wherein step a) results in the dehydrogenation of the alkane and the hydrogenation of the hydrogen acceptor. In such embodiments, the catalytic process is an alkane transfer dehydrogenation process.

It will be appreciated that within the alkane transfer dehydrogenation process, the alkane may be linear or branched, and may be substituted with one or more substituents selected from halo, oxo, hydroxyl and amino. The hydrogen acceptor may be any suitable hydrogen acceptor. Suitably, the hydrogen acceptor is an alkene (e.g. ethene).

In an embodiment, the C₄-C₁₀ hydrocarbon is a C₄-C₅ alkane and the hydrogen acceptor is a C₂-C₆ alkene.

In an embodiment, the C₄-C₁₀ hydrocarbon is butane the hydrogen acceptor is ethene, and where step a) results in the conversion of the butane into 1-butene or 2-butene. It will be appreciated that when butane is dehydrogenated to 1-butene, the 1-butene may subsequently undergo isomerisation to 2-butene (as described above).

In an embodiment, step a) of the transfer dehydrogenation process is conducted at a temperature of 0-100° C. Suitably, step a) is conducted at a temperature of 0-50° C. More suitably, step a) is conducted at a temperature of 0-30° C. Most suitably, step a) is conducted at a temperature of 18-30° C.

In an embodiment, the catalytic process is an alkane transfer dehydrogenation process, wherein the molar ratio of the C₄-C₁₀ hydrocarbon to the hydrogen acceptor is 0.1:1 to 1:6. Suitably, the molar ratio of the C₄-C₁₀ hydrocarbon to the hydrogen acceptor is 1:1 to 1:6. More suitably, molar ratio of the C₄-C₁₀ hydrocarbon to the hydrogen acceptor is 1:1.5 to 1:2.5

In another embodiment, step a) results in the dimerization of two molecules of the C₄-C₁₀ hydrocarbon, wherein the C₄-C₁₀ hydrocarbon is an alkene. In such embodiments, the catalytic process is an alkene dimerization process.

In an embodiment, the C₄-C₁₀ hydrocarbon is a C₂-C₅ alkene. Suitably, the C₄-C₁₀ hydrocarbon is ethene and the process results in the generation of 1-butene and/or 2-butene.

### Compounds of the Invention

As described hereinbefore, the present invention also provides a compound having a structure according to formula (Ia) shown below:

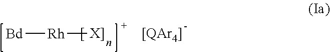

wherein
Bd is a bidentate ligand bonded to Rh via two heteroatoms independently selected from P, N and S,
   wherein the two heteroatoms are independently optionally substituted with one or more substituents selected from iso-propyl, tert-butyl, sec-butyl, n-pentyl, iso-pentyl, neo-pentyl, tert-pentyl, sec-pentyl, 3-pentyl, iso-propoxy, iso-butoxy, tert-butoxy, sec-butoxy, n-pentoxy, iso-pentoxy, neo-pentoxy, tert-pentoxy, sec-pentoxy, 3-pentoxy, 6-8 membered carbocyclyl, 6-8 membered heterocyclyl, aryl or adamantyl, any of which may be optionally substituted with one or more substituents selected from halo, oxo, hydroxyl, (1-4C)alkyl, (2-4C)alkenyl, (2-4C)alkynyl, (1-4C)alkoxy, (1-4C)haloalkyl and —N(Rₓ)(R_{y}),
      wherein Rₓ and R_{y} are each independently selected from hydrogen and (1-4C)alkyl;
each X is independently a ligand that is weakly bound to Rh via one or more bond, wherein the total energy of coordination of Rh to each X is <130 KJmol⁻¹, and wherein each X is selected from hydrogen, dinitrogen, a linear or branched (2-10C)alkene, a 5-10 membered cycloalkene, a linear or branched (6-10C)alkane and a 8-10 membered cycloalkane, any of which may be optionally substituted with one or more substituents selected from halo, oxo, hydroxyl, (1-4C)alkyl, (2-4C)alkenyl, (2-4C)alkynyl, (1-4C)alkoxy, (1-4C)haloalkyl and —N(Rᵥ)(R_{w}),
   wherein Rᵥ and R_{w} are each independently selected from hydrogen and (1-4C)alkyl;
n is 1, 2 or 3;
Q is selected from B, Al, In and Ga; and
each Ar is independently
   i. a phenyl group substituted with one or more substituents selected from halo, (1-3C)alkyl and (1-3C)haloalkyl, or
   ii. a (1-3C)alkoxy group substituted with one or more substituents selected from halo, (1-3C)alkyl and (1-3C)haloalkyl.

As alluded to hereinbefore, although various heterogeneous solidgas catalytic systems for catalytic processes involving C—H bond activation (e.g. alkane dehydrogenation and alkene isomerization) are known,^{27, 35, 36} they often require high temperatures for their operation. Industrially, this is sub-optimal for a variety of reasons. Not only does the requirement for high temperatures have environmental consequences, but the elevated temperatures can themselves shorten the lifetime of the catalyst by thermally-induced decomposition (e.g. by coking). Hence, the poor recyclability of such catalysts, coupled to the high temperatures required for their operation, can result in high operating costs on an industrial scale.

When compared with prior art catalysts useful in catalytic processes involving C—H bond activation the compounds of the invention offer a number of advantages. Chiefly, the compounds of formula (Ia) have been demonstrated to be catalytically active in catalytic processes involving C—H bond activation at temperatures significantly lower than currently available techniques. In particular, the compounds of formula (Ia) have been shown to exhibit significant catalytic activity in alkene isomerisation, alkane transfer dehydrogenation, and alkene dimerization reactions at room temperature. Moreover, the compounds of formula (Ia) exhibit remarkable long-term stability, as well as notable catalytic recyclability.

In an embodiment, Bd is a bidentate ligand bonded to Rh via two heteroatoms independently selected from P, N and S, wherein the two heteroatoms are independently optionally substituted with one or more substituents selected from iso-propyl, tert-butyl, sec-butyl, n-pentyl, iso-pentyl, neo-pentyl, tert-pentyl, sec-pentyl, 3-pentyl, iso-propoxy, iso-butoxy, tert-butoxy, sec-butoxy, n-pentoxy, iso-pentoxy, neo-pentoxy, tert-pentoxy, sec-pentoxy, 3-pentoxy, 6-8 membered carbocyclyl, 6-8 membered heterocyclyl, aryl or adamantyl, any of which may be optionally substituted with one or more substituents selected from halo, oxo, hydroxyl, (1-4C)alkyl, (2-4C)alkenyl, (2-4C)alkynyl, (1-4C)alkoxy, (1-4C)haloalkyl and —N(Rₓ)(R_{y}),

wherein Rₓ and R_{y} are each independently selected from hydrogen and (1-4C)alkyl;
each X is independently a ligand that is weakly bound to Rh via one or more bond, wherein the total energy of coordination of Rh to each X is <130 KJmol⁻¹, and wherein each X is selected from dinitrogen, a linear or branched (2-10C)alkene, a 5-10 membered cycloalkene, a linear or branched (6-10C)alkane and a 8-10 membered cycloalkane, any of which may be optionally substituted with one or more substituents selected from halo, oxo, hydroxyl, (1-4C)alkyl, (2-4C)alkenyl, (2-4C)alkynyl, (1-4C)alkoxy, (1-4C)haloalkyl and —N(Rᵥ)(R_{w}),

wherein Rᵥ and R_{w} are each independently selected from hydrogen and (1-4C)alkyl;
n is 1, 2 or 3;
Q is selected from B, Al and In; and
each Ar is independently a phenyl group substituted with one or more substituents selected from (1-3C)alkyl and (1-3C)haloalkyl.

In an embodiment, the two heteroatoms of Bd are independently substituted with one or more substituents selected from iso-propyl, tert-butyl, sec-butyl, iso-propoxy, tert-butoxy, sec-butoxy, 6-8 membered carbocyclyl, 6-8 membered heterocyclyl (e.g. tetrahydropyranyl), aryl or adamantyl, any of which may be optionally substituted with one or more substituents selected from halo, hydroxyl, (1-4C)alkyl, (1-4C)alkoxy, and —N(Rₓ)(R_{y}),

wherein Rₓ and R_{y} are each independently selected from hydrogen and (1-4C)alkyl.

In an embodiment, the two heteroatoms of Bd are independently substituted with one or more substituents selected from iso-propyl, tert-butyl, 6-8 membered carbocyclyl, aryl or adamantyl, any of which may be optionally substituted with one or more substituents selected from halo, hydroxyl, (1-4C)alkyl and (1-4C)alkoxy.

In an embodiment, the two heteroatoms of Bd are independently substituted with one or more substituents selected from iso-propyl, 6-8 membered carbocyclyl, aryl or adamantyl, any of which may be optionally substituted with one or more substituents selected from halo, hydroxyl, (1-4C)alkyl and (1-4C)alkoxy.

In an embodiment, the two heteroatoms of Bd are independently substituted with one or more substituents selected from iso-propyl, cyclohexyl or aryl, any of which may be optionally substituted with one or more substituents selected from halo, hydroxyl, (1-4C)alkyl and (1-4C)alkoxy.

In an embodiment, the two heteroatoms of Bd are independently substituted with one or more cyclohexyl substituents, any of which may be substituted with one or more substituents selected from halo, hydroxyl, (1-4C)alkyl and (1-4C)alkoxy.

In an embodiment, the two heteroatoms of Bd are each substituted with two cyclohexyl substituents.

In an embodiment, Bd is a bis-phosphine or bis-amine bidentate ligand.

In an embodiment, Bd is a bis-amine bidentate ligand.

In an embodiment, Bd is a bis-amine bidentate ligand selected from ethylenediamine, 1,4-diazadiene, 1,1′-bipyridine, 1,10-phenanthroline and ethylenediaminetetraacetate, wherein one or more of the N atoms is independently optionally substituted with one or more substituents as defined hereinbefore in respect of Bd.

In an embodiment, Bd is a bis-phosphine bidentate ligand. The bis-phosphine bidentate ligand may have a structure according to formula (IIa) shown below:

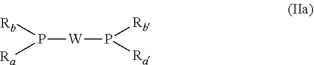

wherein

Rₐ, Rₐ′, R_{b} and R_{b}′ are each independently iso-propyl, tert-butyl, sec-butyl, n-pentyl, iso-pentyl, neo-pentyl, tert-pentyl, sec-pentyl, 3-pentyl, iso-propoxy, tert-butoxy, sec-butoxy, n-pentoxy, iso-pentoxy, neo-pentoxy, tert-pentoxy, sec-pentoxy, 3-pentoxy, 6-8 membered carbocyclyl, 6-8 membered heterocyclyl, aryl or adamantyl, any of which may be optionally substituted with one or more substituents selected from halo, oxo, hydroxyl, (1-4C)alkyl, (2-4C)alkenyl, (2-4C)alkynyl, (1-4C)alkoxy, (1-4C)haloalkyl and —N(Rₓ)(R_{y}),
wherein Rₓ and R_{y} are each independently selected from hydrogen and (1-4C)alkyl; and

W is a (1-5C)alkylene linking group, wherein one or more of the carbon atoms may be replaced with a heteroatom selected from N, O and S, and wherein W is optionally substituted with one or more groups R_{c},
wherein each R_{c} is independently selected from halo, oxo, hydroxyl, (1-4C)alkyl, (2-4C)alkenyl, (2-4C)alkynyl, (1-4C)alkoxy, (1-4C)haloalkyl and aryl,
and/or two groups R_{c} may be linked, such that when taken with the atoms to which they are attached, they form a phenyl group optionally fused to a phenyl or 5-6-membered heteroaryl, wherein any or all of the rings are optionally substituted with one or more substituents selected from halo and (1-4C)alkyl.

In an embodiment, the bis-phosphine bidentate ligand has a structure according to formula (IIa), wherein W is a (1-5C)alkylene linking group optionally substituted with one or more groups R_{c}, wherein each R_{c} is independently (1-4C)alkyl or (1-4C)alkoxy, and/or two groups R_{c} may be linked, such that when taken with the atoms to which they are attached, they form a phenyl group optionally substituted with one or more substituents selected from halo, (1-4C)alkyl and (1-4C)alkoxy.

In an embodiment, the bis-phosphine bidentate ligand has a structure according to formula (IIa), wherein W is a (1-5C)alkylene linking group optionally substituted with one or more groups R_{c}, wherein each R_{c} is independently (1-4C)alkyl, and/or two groups R_{c} may be linked, such that when taken with the atoms to which they are attached, they form a phenyl group optionally substituted with one or more substituents selected from halo and (1-4C)alkyl.

In an embodiment, the bis-phosphine bidentate ligand has a structure according to formula (II), wherein W has any of the following structures:

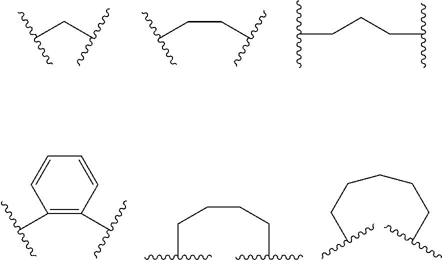

In an embodiment, the bis-phosphine bidentate ligand has a structure according to formula (IIa), wherein W has any of the following structures:

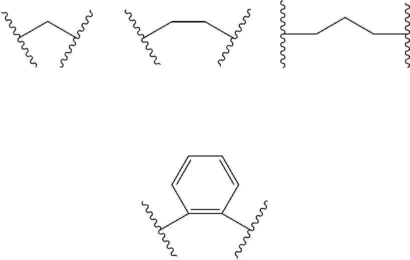

In an embodiment, the bis-phosphine bidentate ligand has a structure according to formula (II), wherein W is ethylene, propylene, butylene or pentylene.

In an embodiment, the bis-phosphine bidentate ligand has a structure according to formula (IIa), wherein W is ethylene.

In an embodiment, the bis-phosphine bidentate ligand has a structure according to formula (IIa), wherein Rₐ, Rₐ′, R_{b} and R_{b}′ are each independently iso-propyl, tert-butyl, sec-butyl, iso-propoxy, tert-butoxy, sec-butoxy, 6-8 membered carbocyclyl, 6-8 membered heterocyclyl (e.g. tetrahydropyranyl), aryl or adamantyl, any of which may be optionally substituted with one or more substituents selected from halo, hydroxyl, (1-4C)alkyl, (1-4C)alkoxy, and —N(Rₓ)(R_{y}),

wherein Rₓ and R_{y} are each independently selected from hydrogen and (1-4C)alkyl.

In an embodiment, the bis-phosphine bidentate ligand has a structure according to formula (IIa), wherein Rₐ, Rₐ′, R_{b} and R_{b}′ are each independently iso-propyl, tert-butyl, 6-8 membered carbocyclyl, aryl or adamantyl, any of which may be optionally substituted with one or more substituents selected from halo, hydroxyl, (1-4C)alkyl and (1-4C)alkoxy.

In an embodiment, the bis-phosphine bidentate ligand has a structure according to formula (IIa), wherein Rₐ, Rₐ′, R_{b} and R_{b}′ are each independently iso-propyl, 6-8 membered carbocyclyl, aryl or adamantyl, any of which may be optionally substituted with one or more substituents selected from halo, hydroxyl, (1-4C)alkyl and (1-4C)alkoxy.

In an embodiment, the bis-phosphine bidentate ligand has a structure according to formula (IIa), wherein Rₐ, Rₐ′, R_{b} and R_{b}′ are each independently iso-propyl, cyclohexyl or aryl, any of which may be optionally substituted with one or more substituents selected from halo, hydroxyl, (1-4C)alkyl and (1-4C)alkoxy.

In an embodiment, the bis-phosphine bidentate ligand has a structure according to formula (IIa), wherein Rₐ, Rₐ′, R_{b} and R_{b}′ are cyclohexyl, any of which may be substituted with one or more substituents selected from halo, hydroxyl, (1-4C)alkyl and (1-4C)alkoxy.

In an embodiment, the bis-phosphine bidentate ligand has a structure according to formula (IIa), wherein Rₐ, Rₐ′, R_{b} and R_{b}′ are cyclohexyl.

Each X is a weakly bound ligand. It will be appreciated by those of skill in the art that the strength of binding between Rh and X has important implications for the catalytic activity of the compounds. In particular, it will be appreciated that a weakly bound ligand X is one that can be displaced by the C₄-C₁₀ hydrocarbon used during step a) of the catalytic process of the invention. In an embodiment, each X is independently a ligand weakly bound to Rh via one or more bond, wherein the total energy of coordination of Rh to each X is 5-130 KJ mol⁻¹. More suitably, each X is independently a ligand weakly bound to Rh via one or more bond, wherein the total energy of coordination of Rh to each X is 5-125 KJ mol⁻¹. Yet more suitably, each X is independently a ligand weakly bound to Rh via one or more bond, wherein the total energy of coordination of Rh to each X is 5-122 KJ mol⁻¹. Most suitably, each X is independently a ligand weakly bound to Rh via one or more bond, wherein the total energy of coordination of Rh to each X is 5-118 KJ mol⁻¹.

In an embodiment, each X is hydrogen, an alkane, an alkene or dinitrogen.

In an embodiment, each X is an alkane, an alkene or dinitrogen.

In an embodiment, each X is selected from hydrogen, dinitrogen, a linear or branched (2-10C)alkene, a 5-10 membered cycloalkene, a linear or branched (6-10C)alkane and a 8-10 membered cycloalkane, any of which may be optionally substituted with one or more substituents selected from halo, oxo, hydroxyl, (1-4C)alkyl, (2-4C)alkenyl, (2-4C)alkynyl, (1-4C)alkoxy, (1-4C)haloalkyl and N(Rₓ)(R_{y}),

wherein Rₓ and R_{y} are each independently selected from hydrogen and (1-4C)alkyl

In an embodiment, each X is selected from hydrogen, dinitrogen, a linear or branched (2-10C)alkene, a monounsaturated 5-10 membered cycloalkene, a branched (6-10C)alkane and a 8-10 membered cycloalkane, any of which may be optionally substituted with one or more substituents selected from halo, oxo, hydroxyl, (1-4C)alkyl, (2-4C)alkenyl, (2-4C)alkynyl, (1-4C)alkoxy and (1-4C)haloalkyl.

In an embodiment, each X is selected from hydrogen, dinitrogen, a linear or branched (2-8C)alkene, a monounsaturated 5-8 membered cycloalkene, a branched (6-10C)alkane and a 8-10 membered cycloalkane, any of which may be optionally substituted with one or more substituents selected from halo, oxo, hydroxyl, (1-4C)alkyl, (2-4C)alkenyl, (2-4C)alkynyl, (1-4C)alkoxy and (1-4C)haloalkyl.

In an embodiment, each X is selected from dinitrogen, a linear or branched (2-10C)alkene, a 5-10 membered cycloalkene, a linear or branched (6-10C)alkane and a 8-10 membered cycloalkane, any of which may be optionally substituted with one or more substituents selected from halo, oxo, hydroxyl, (1-4C)alkyl, (2-4C)alkenyl, (2-4C)alkynyl, (1-4C)alkoxy, (1-4C)haloalkyl and —N(Rₓ)(R_{y}),

wherein Rₓ and R_{y} are each independently selected from hydrogen and (1-4C)alkyl

In an embodiment, each X is selected from dinitrogen, a linear or branched (2-10C)alkene, a monounsaturated 5-10 membered cycloalkene, a branched (6-10C)alkane and a 8-10 membered cycloalkane, any of which may be optionally substituted with one or more substituents selected from halo, oxo, hydroxyl, (1-4C)alkyl, (2-4C)alkenyl, (2-4C)alkynyl, (1-4C)alkoxy and (1-4C)haloalkyl.

In an embodiment, each X is selected from dinitrogen, a linear or branched (2-8C)alkene, a monounsaturated 5-8 membered cycloalkene, a branched (6-10C)alkane and a 8-10 membered cycloalkane, any of which may be optionally substituted with one or more substituents selected from halo, oxo, hydroxyl, (1-4C)alkyl, (2-4C)alkenyl, (2-4C)alkynyl, (1-4C)alkoxy and (1-4C)haloalkyl.

Exemplary linear alkenes include ethene, propene, butene and hexene.

Exemplary 5-10 membered cycloalkenes include cycloheptene and cyclooctene.

Exemplary 8-10 membered cycloalkanes are illustrated below:

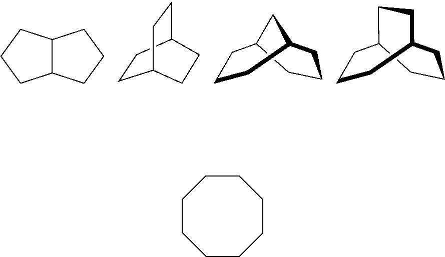

In an embodiment, each X is selected from hydrogen, ethene, propene, butane, hexane and cyclooctene.

In an embodiment, each X is selected from ethene, propene, butene and hexene. Suitably, each X is ethene.

It will be understood that the nature of bonding between Rh and X will depend on the nature of X. When X is ethene, each ethene ligand may be rᵢg coordinated to Rh. When X is an alkane, the alkane ligand is coordinated to Rh by a 3-centre 2-electron sigma interaction between the CH bond of the alkane and the metal centre.

It will be understood that the value of n depends on the nature of X. For smaller X ligands (e.g. hydrogen and ethene), Rh can accommodate two or three X ligands (e.g. n=2 or 3). For larger X ligands (e.g. butane), Rh can accommodate only one X ligand (e.g. n=1). Suitably, n is 1 or 2.

In an embodiment, Q is boron or aluminium.

In an embodiment, Q is boron.

In an embodiment, each Ar is either i) a phenyl group substituted at the 3-, 4- and/or 5-position with one or more substituents selected from halo (1-3C)alkyl and (1-3C)haloalkyl, or ii) a (1-3C)alkoxy group substituted with one or more substituents selected from halo (1-3C)alkyl and (1-3C) haloalkyl.

In an embodiment, each Ar is either i) a phenyl group substituted at the 3-, and/or 5-position with one or more substituents selected from fluoro, chloro, (1-3C)alkyl and (1-3C)haloalkyl, or ii) a (1-3C)alkoxy group substituted with one or more substituents selected from fluoro, chloro and (1-2C)haloalkyl.

In an embodiment, each Ar is either i) a phenyl group substituted at the 3-, and/or 5-position with one or more substituents selected from fluoro, chloro, (1-2C)alkyl and (1-2C)fluoroalkyl, or ii) a (1-2C)alkoxy group substituted with one or more substituents selected from fluoro, chloro and (1-2C)haloalkyl.

In an embodiment, each Ar is a phenyl group substituted at the 3-, 4- and/or 5-position with one or more substituents selected from (1-3C)alkyl and (1-3C)haloalkyl.

In an embodiment, each Ar is a phenyl group substituted at the 3- and/or 5-position with one or more substituents selected from (1-3C)alkyl and (1-3C)haloalkyl.

In an embodiment, each Ar is a phenyl group substituted at the 3- and/or 5-position with one or more substituents selected from (1-2C)alkyl and (1-2C)fluoroalkyl.

In an embodiment, each Ar is a phenyl group substituted at both the 3- and 5-position with a substituent selected from (1-2C)alkyl and (1-2C)fluoroalkyl.

In an embodiment, each Ar is a phenyl group substituted at both the 3- and 5-position with trifluoromethyl.

In an embodiment, [QAr₄] has any of the following structures:

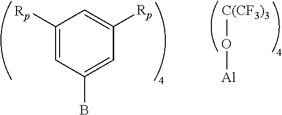

wherein Rₚ is fluoro, chloro, difluoromethyl or trifluromethyl. Suitably, Rₚ is fluoro, chloro or trifluromethyl.

In a particular embodiment, the compound of formula (I) has any of the following structures:

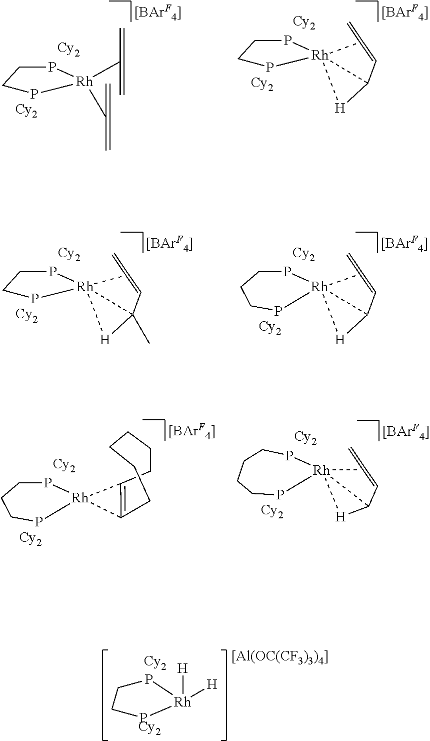

wherein ‘Cy’ denotes cyclohexyl and ‘Ar^{F}’ denotes 3,5-(CF₃)₂C₆H₃.

In a particular embodiment, the compound of formula (Ia) has any of the following structures:

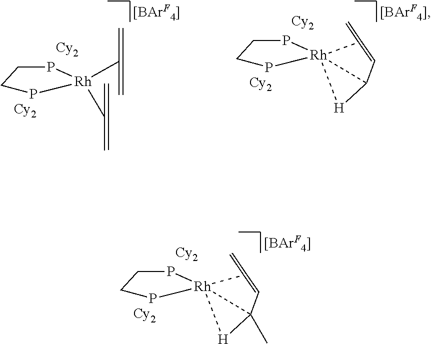

wherein ‘Cy’ denotes cyclohexyl and ‘Ar^{F}’ denotes 3,5(CF₃)₂C₆H₃.

In a particular embodiment, the compound of formula (Ia) has either of the following structures:

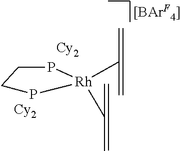

wherein ‘Cy’ denotes cyclohexyl and ‘Ar^{F}’ denotes 3,5-(CF₃)₂C₆H₃.

In an embodiment, the compound of formula (Ia) is a solid. Suitably, the compound of formula (Ia) is crystalline.

In an embodiment, the compound of formula (Ia) is unsupported. By virtue of their crystalline morphology, the compounds of formula (Ia) are themselves suitable for direct use in heterogeneous catalytic systems, without the need for being supported on a separate solid support (e.g. silica or alumina).

In an embodiment, the compound of formula (Ia) is

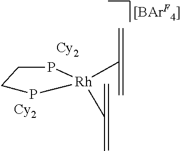

wherein ‘Cy’ denotes cyclohexyl and ‘Ar^{F}’ denotes 3,5-(CF₃)₂C₆H₃, and wherein the compounds has octahedral crystal morphology. The space groups is C2/c (No. 15 International Tables). Suitably, the X-ray powder diffraction pattern for the compound exhibits strong peaks at 2theta=9.1953 and 19.1186°.

In an embodiment, the compound of formula (Ia) is

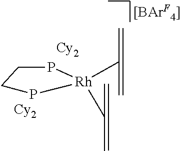

wherein ‘Cy’ denotes cyclohexyl and ‘Ar^{F}’ denotes 3,5-(CF₃)₂C₆H₃, and wherein the compounds has hexagonal crystal morphology. The space groups is P6₃22 (No. 182 International Tables). Suitably, the X-ray powder diffraction pattern for the compound exhibits strong peaks at 2theta=3.9514 and 6.8133°.

In another aspect, the present invention provides a compound having a structure according to formula (Ia) described hereinbefore, wherein Bd, n, Q and Ar have any of the definitions appearing hereinbefore, and each X is independently a ligand that is weakly bound to Rh via one or more bond, each bond having a bond energy of <130 KJmol⁻¹, with the proviso that X is not norbornane or n-pentane.

### Preparation of Compounds of Invention

The compounds of the invention can be prepared by any suitable means known in the art.

In one aspect, the compounds of formula (Ia) are prepared by a process comprising the following steps:
a) providing a compound having a structure according to formula (Ia′) below:

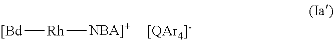

wherein
Bd, Q and Ar are as defined for formula (Ia); and
NBA is norbornane,
b) contacting the compound of formula (Ia′) with a ligand X as defined in respect of formula (Ia).

It will be appreciated that Bd, Q, Ar and X may have any of the definitions appearing hereinbefore in respect of the compounds of formula (Ia).

Suitably, the compound of formula (Ia′) is a solid, and step b) is conducted in the solid phase (i.e. not in solution). More suitably, in step b), X is provided as a gas.

In a particular embodiment, the compound of formula (Ia) is:

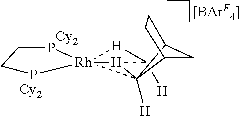

wherein ‘Cy’ denotes cyclohexyl and ‘Ar^{F}’ denotes 3,5-(CF₃)₂C₆H₃;

and step b) comprises contacting the compound of formula (Ia′) with ethene. Such a process results in the formation of [1-(ethene)₂][BAr^{F}₄] having octahedral crystal morphology.

In a particular embodiment, the compound of formula (Ia) is:

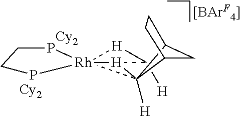

wherein ‘Cy’ denotes cyclohexyl and ‘Ar^{F}’ denotes 3,5-(CF₃)₂C₆I⁻1₃;
step b) comprises contacting the compound of formula (Ia′) with ethene; and
the process further comprises a step c) of recrystallizing the product resulting from step b) from a mixture of CH₂Cl₂ and pentane under an atmosphere of ethene at a temperature of −80° C. Such a process results in the formation of [1-(ethene)₂][BAr^{F}₄] having hexagonal crystal morphology.

The person skilled in the art will be able to select appropriate reaction conditions (e.g. temperatures, pressures, and durations) for carrying out the processes described herein.

In another aspect, the present invention provides a compound of formula (Ia) obtainable, obtained or directly obtained by a process described herein.

### EXAMPLES

Examples of the invention will now be described, for the purpose of illustration only, with reference to the accompanying figures, in which:

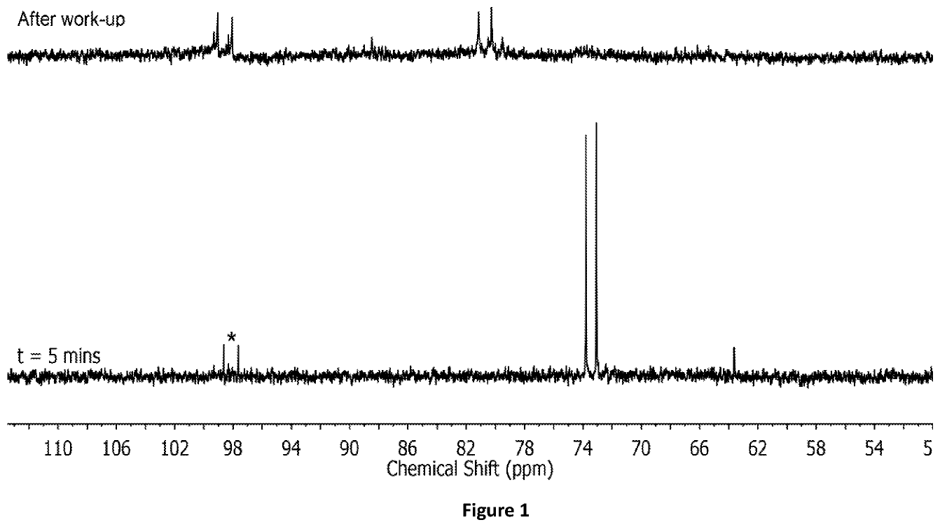

FIG. 1 shows the ³¹ Pe H} solution NMR spectrum of the attempted solution phase synthesis of [1-(ethene)₂][BAr^{F}₄]. The bottom spectrum is measured after 5 minutes reaction, the top spectrum is after attempted work up.

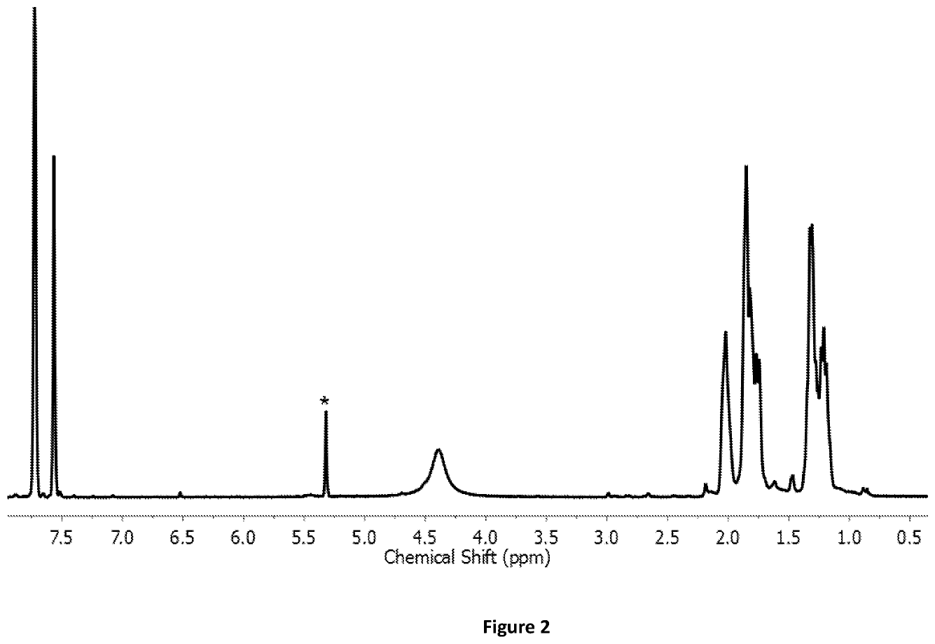

FIG. 2 shows the solution ¹H NMR spectrum (CD₂Cl₂) of [1-(ethene)₂][BAr^{F}₄] (from dissolved [1-(ethene)₂][BAr^{F}₄]-Oct (where “Oct” refers to the C2/c space group material) measured at room temperature.

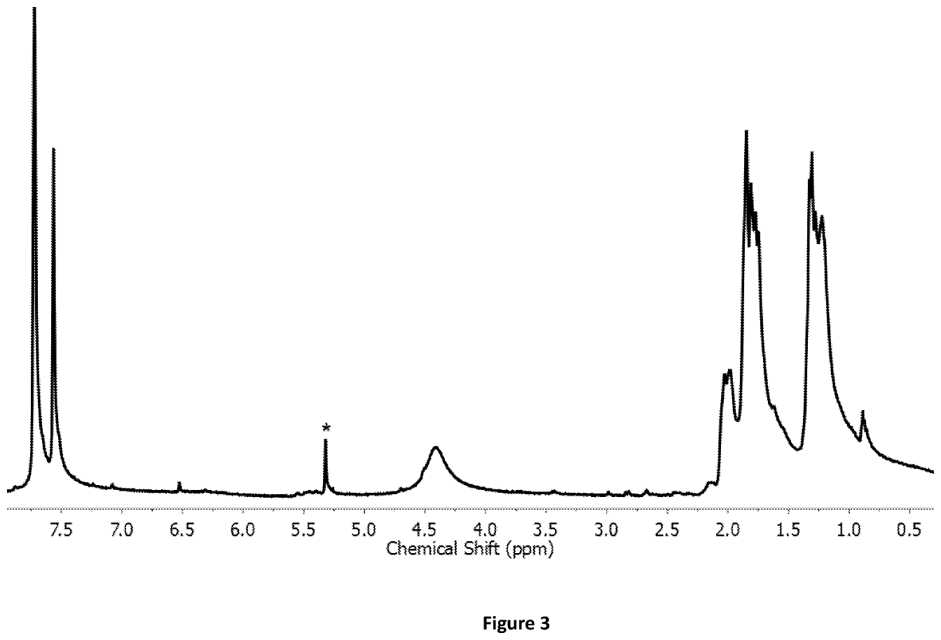

FIG. 3 shows the solution ¹H NMR spectrum (CD₂Cl₂) of [1-(ethene)₂][BAr^{F}₄] (from dissolved [1-(ethene)₂][BAr^{F}₄]-Hex, where “Hex” refers to the P6₃22 space group material) measured at room temperature. The resonance marked * is due to residual protio solvent.

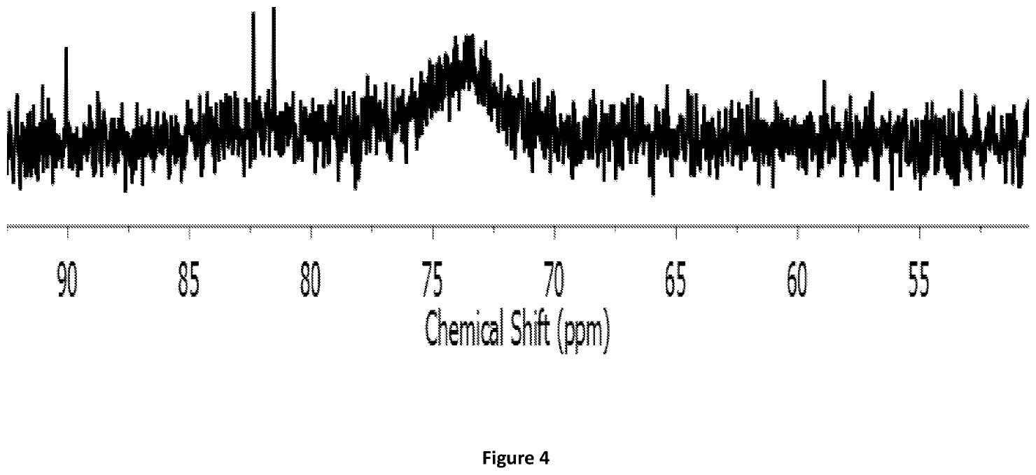

FIG. 4 shows the solution ³¹P{¹H} NMR spectrum (CD₂Cl₂) of [1-(ethene)₂][BAr^{F}₄] (from dissolved [1-(ethene)₂][BAr^{F}₄]-Oct measured at room temperature.

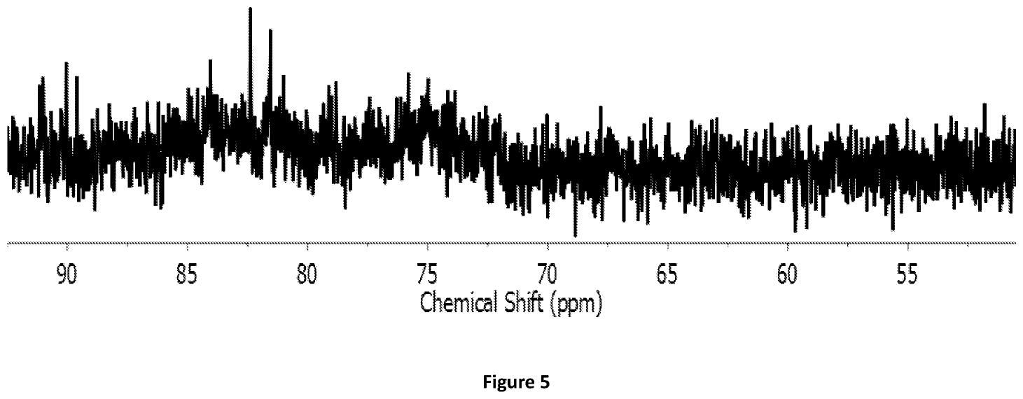

FIG. 5 shows the solution ³¹ Pe ¹H} NMR spectrum (CD₂Cl₂) of [1-(ethene)₂][BAr^{F}₄] (from dissolved [1-(ethene)₂][BAr^{F}₄]-Hex) measured at room temperature.

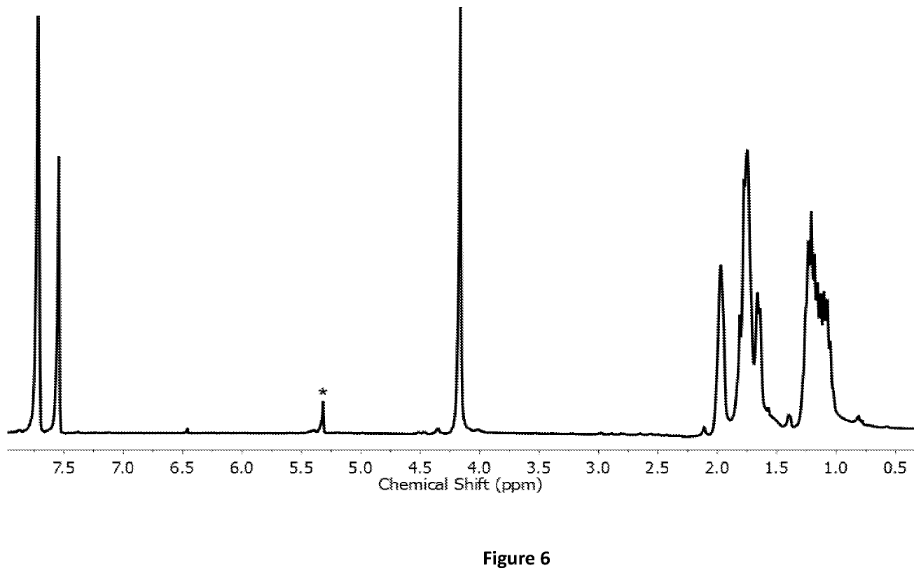

FIG. 6 shows the solution ¹H NMR (CD₂Cl₂) spectrum of [1-(ethene)₂][BAr^{F}₄] (from dissolved 1-(ethene)₂][BAr^{F}₄]-Oct measured at 193 K.

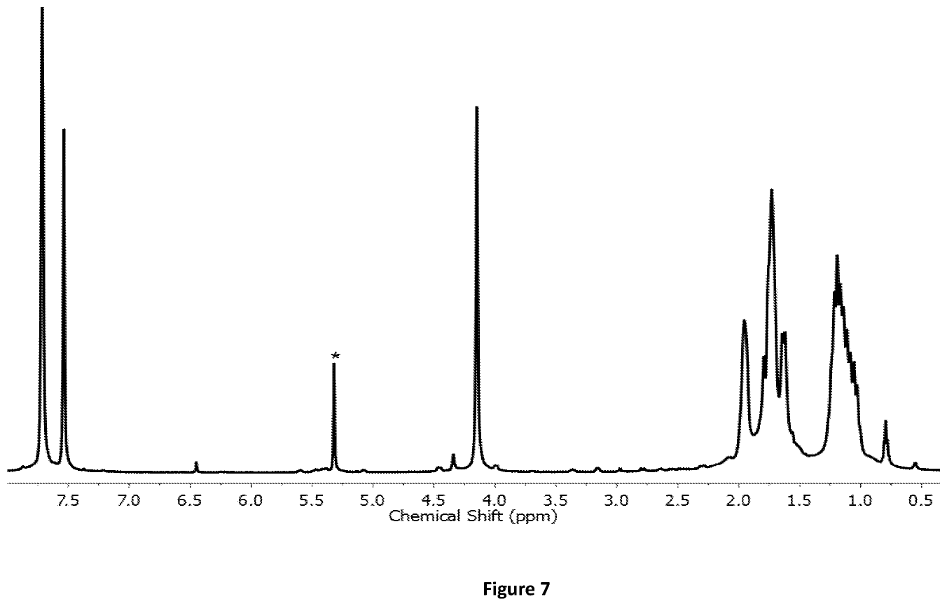

FIG. 7 shows the solution ¹H NMR (CD₂Cl₂) spectrum of [1-(ethene)₂][BAr^{F}₄] (from dissolved [1-(ethene)₂][BAr^{F}₄]-Hex) measured at 193 K.

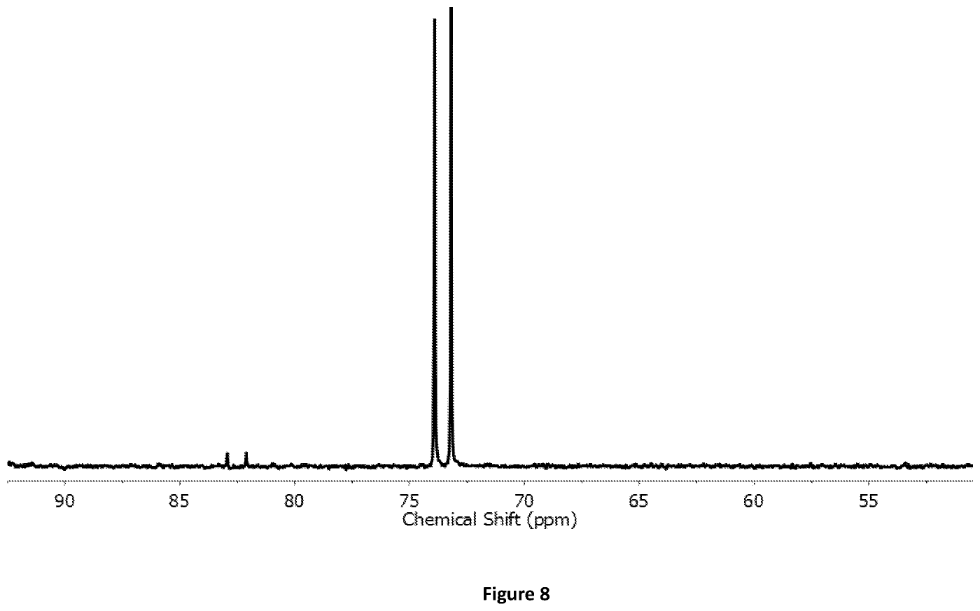

FIG. 8 shows the solution ³¹P{¹H} NMR (CD₂Cl₂) spectrum of 1-(ethene)₂][BAr^{F}₄] (from dissolved [1-(ethene)₂][BAr^{F}₄]-Oct measured at 193 K.

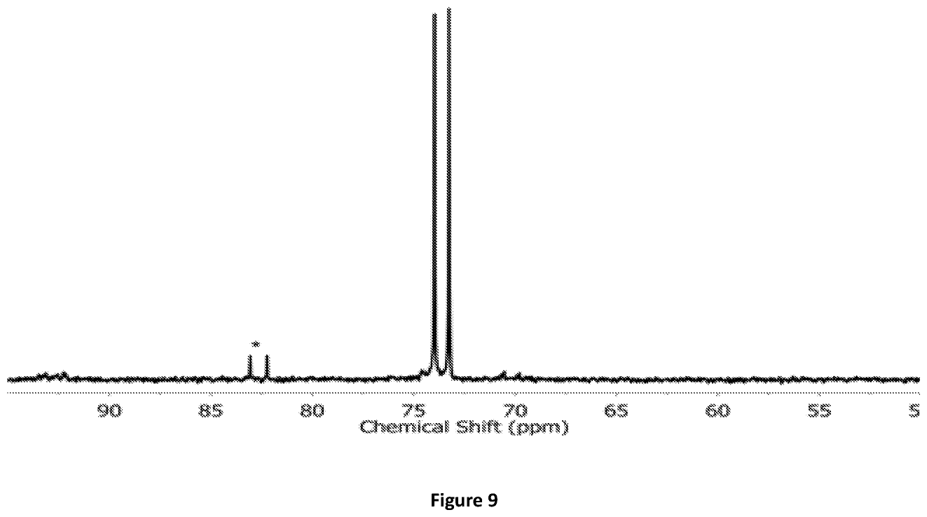

FIG. 9 shows the solution ³¹ P{¹H} NMR (CD₂Cl₂) spectrum of [1-(ethene)₂][BAr^{F}₄] (from dissolved [1-(ethene)₂][BAr^{F}₄]-Hex) measured at 193 K.

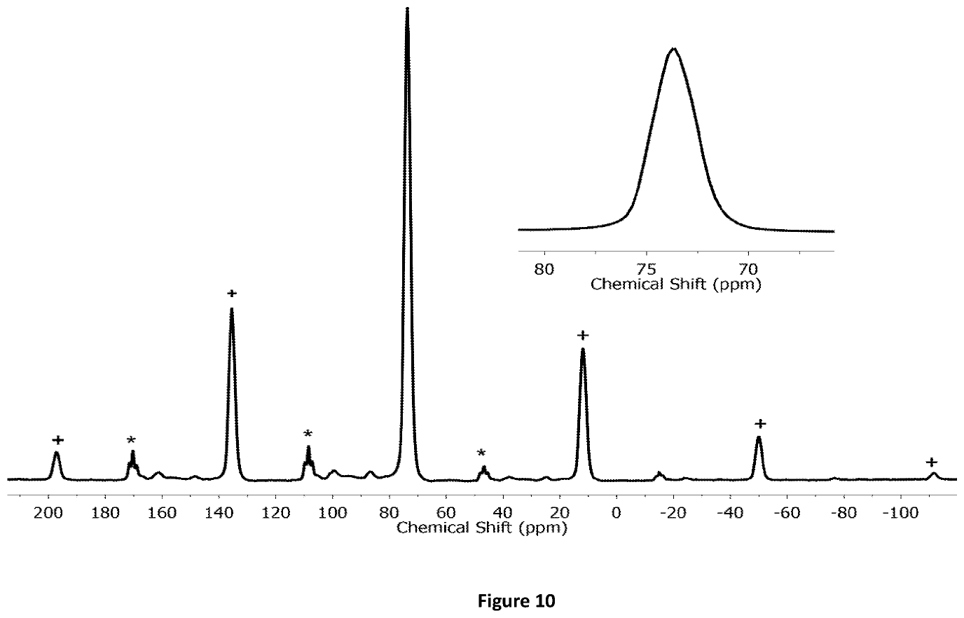

FIG. 10 shows the solid state ³¹P{¹H} NMR of [1-(ethene)₂][BAr^{F}₄]-Oct, made by the exposure of [1-NBA][BAr^{F}₄] to ethylene (1 bar), in a solid state NMR rotor, for 2 hours.

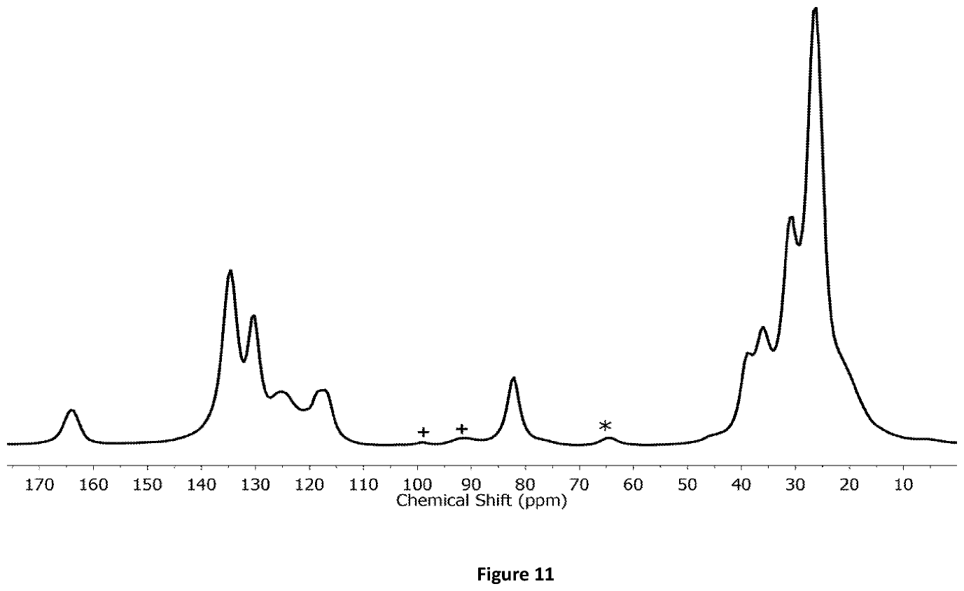

FIG. 11 shows the solid state ¹³C{¹H} NMR of [1-(ethene)₂][BAr^{F}₄]-Oct, made by the exposure of [1-NBA][BAr^{F}₄] to ethylene (2 bar), in a solid state NMR rotor, for 2 hours.

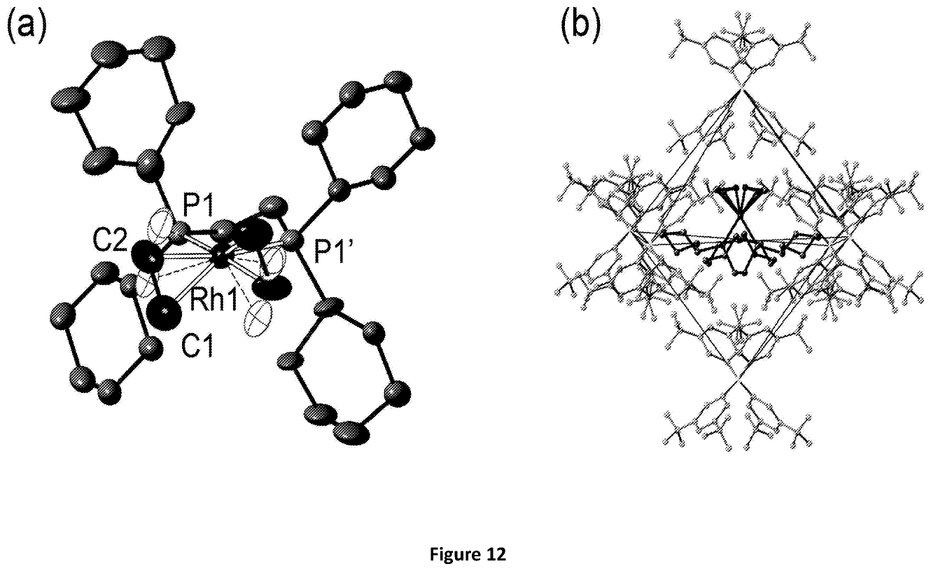

FIG. 12 shows the solidstate structure of [1-(ethene)₂][BAr^{F}₄]Oct. (a) Cation showing the numbering scheme, displacement ellipsoids shown at the 50% probability level, 50% disorder component shown as open ellipsoids. (b) Local environment around the cation showing the arrangement of [BAr^{F}₄]⁻ anions. H-atoms are omitted.

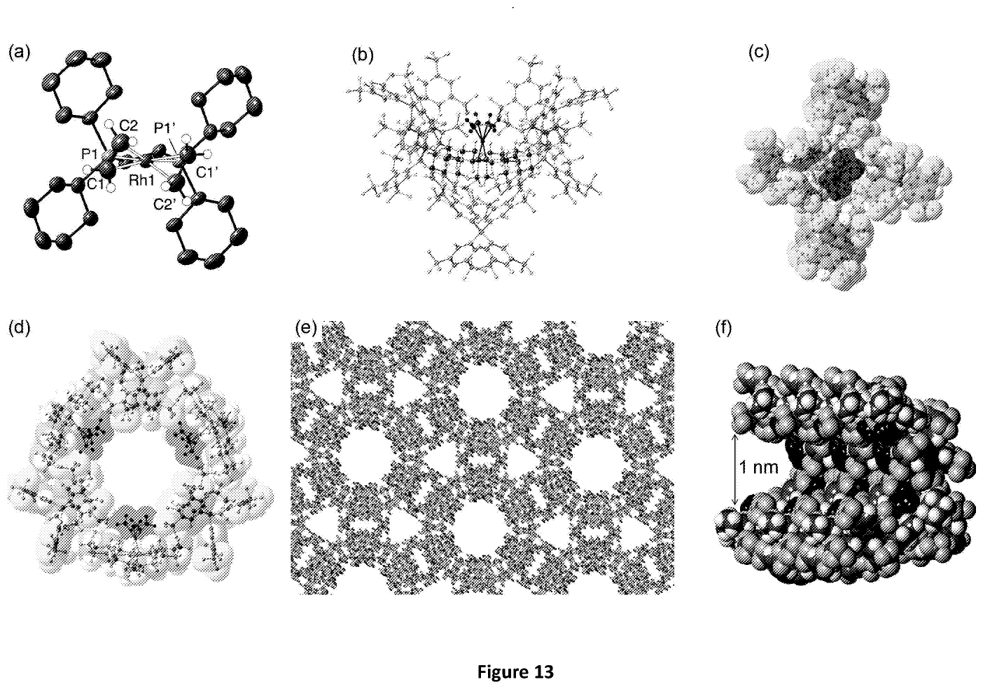

FIG. 13 shows the solidstate structure of [1-(ethene)₂][BAr^{F}₄]-Hex with the ethene groups coloured in red [(b) to (f)] to highlight their positions. (a) Cation showing the numbering scheme, displacement ellipsoids shown at the 50% probability level; (b) Local environment around the cation showing the arrangement of [BAr^{F}₄]⁻ anions; (c) Van der Waals radii spacefilling representation of (b) showing an alternate view highlighting the {Rh(ethene)₂}⁺ fragment; (d) Van der Waals radii spacefilling representation showing the showing the packing arrangement leading to a solventaccessible channel, as viewed down the c-axis; (e) Extended structure viewed down the c-axis; (f) Detail of a channel shown at the Van der Waals radii highlighting the arrangement of {Rh(ethene)₂}⁺ fragments.

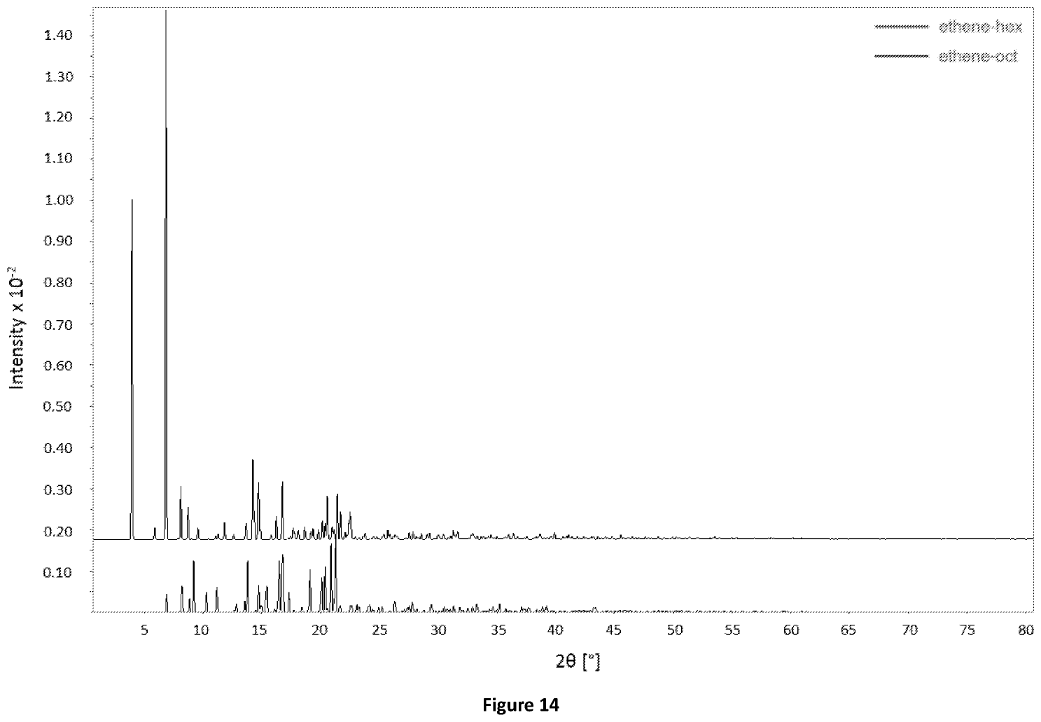

FIG. 14 shows the simulated (as calculated from the single crystal diffraction data using CrystalMaker software) X-ray powder diffraction patterns for [1-(ethene)₂][BAr^{F}₄]Oct and for [1-(ethene)₂][BAr^{F}₄]-Hex.

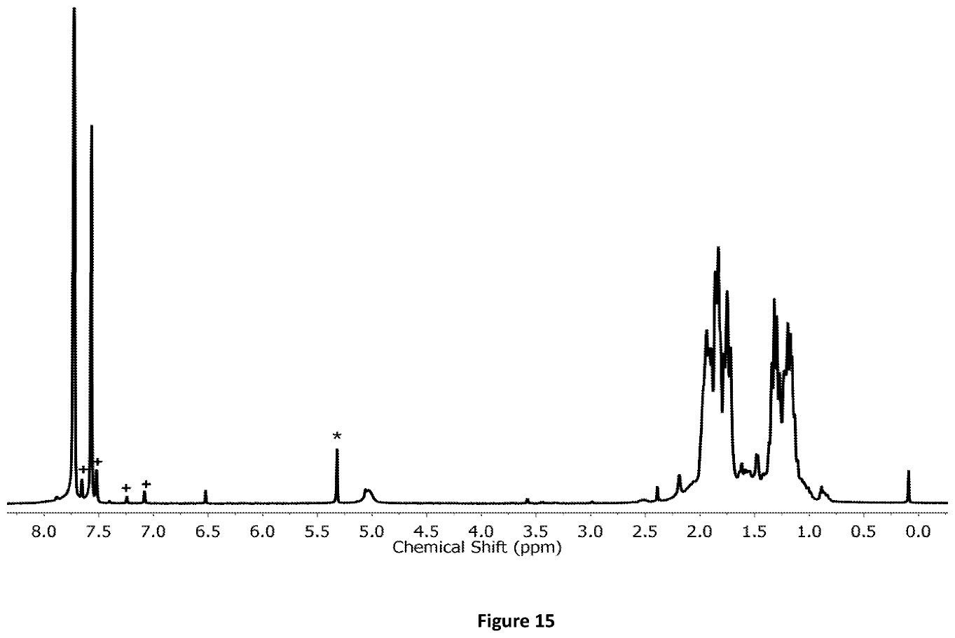

FIG. 15 shows the solution ¹H NMR spectrum (CD₂Cl₂) of [1-propene][BAr^{F}₄], measured at room temperature, measured immediately upon dissolution.

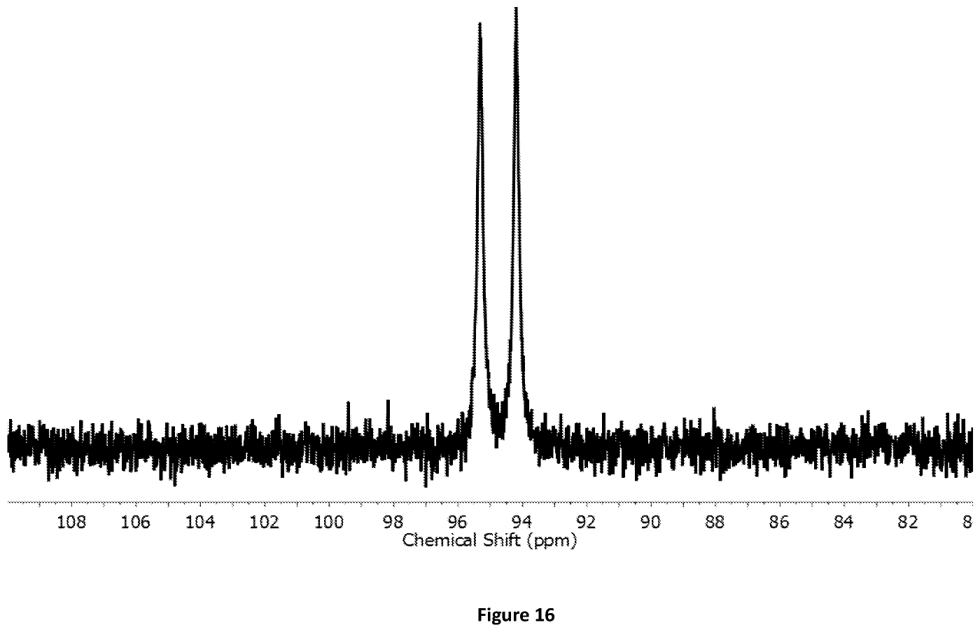

FIG. 16 shows the solution ³¹P{¹H} spectrum of [1-propene][BAr^{F}₄] (CD₂Cl₂) measured at room temperature (immediately upon dissolution).

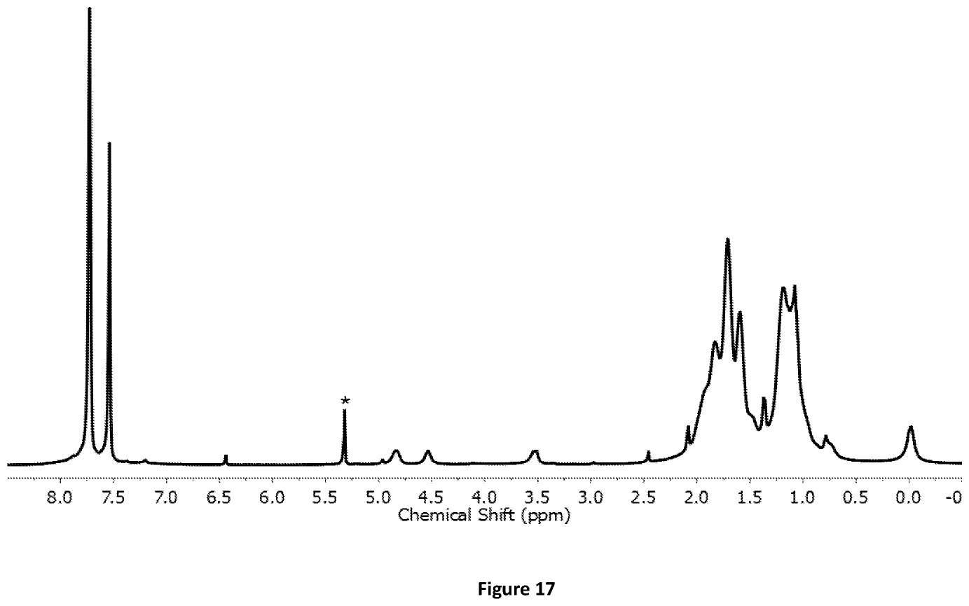

FIG. 17 shows the solution ¹H NMR spectrum of [1-propene][BAr^{F}₄] measured at 193 K, measured immediately on dissolution and using a pre-cooled spectrometer.

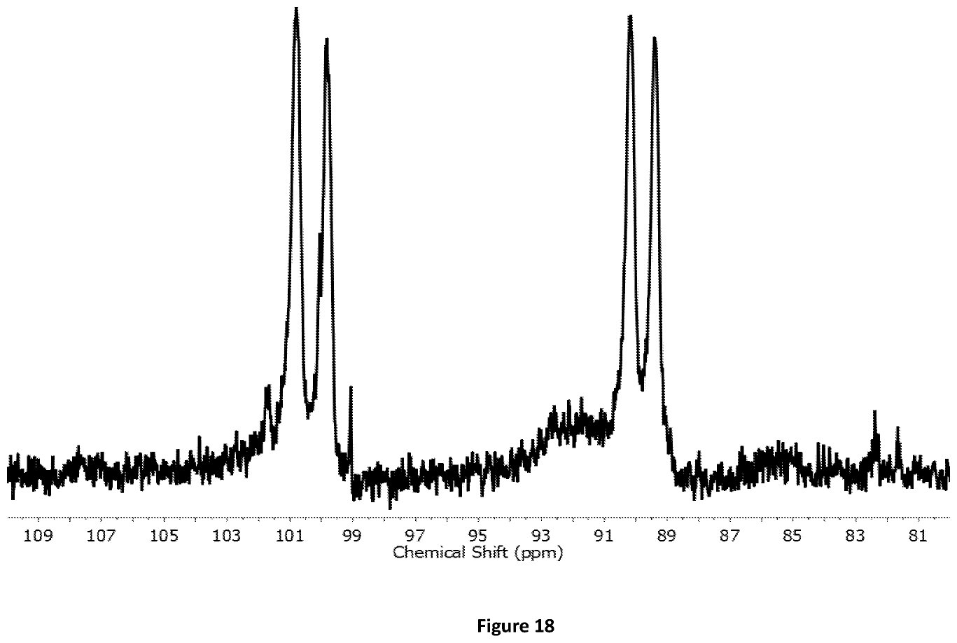

FIG. 18 shows the solution ³¹P{¹H} spectrum of [1-propene][BAr^{F}₄] (CD₂Cl₂) measured at 193 K (immediately upon dissolution).

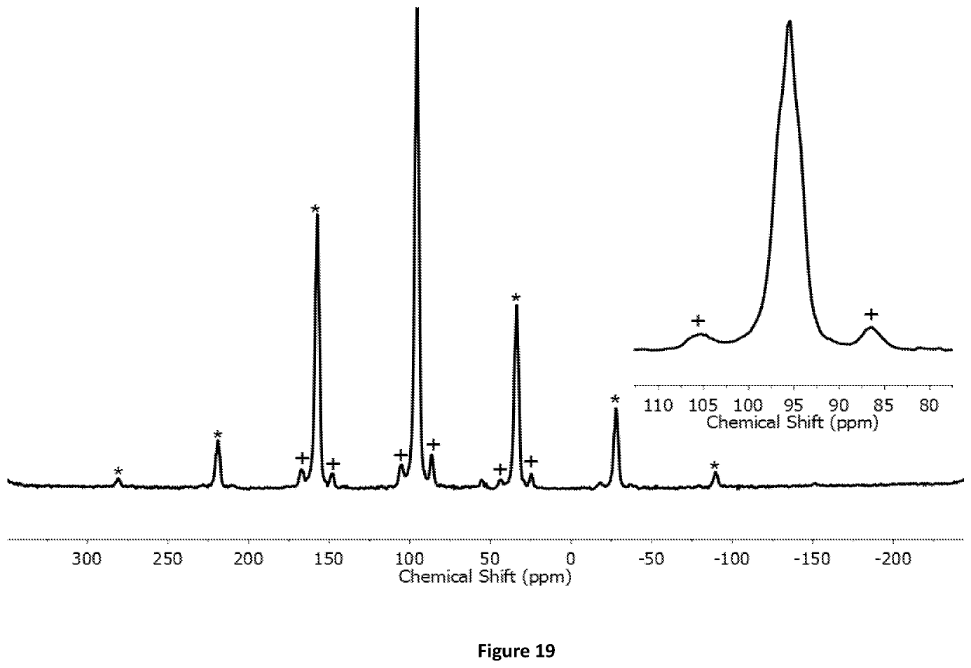

FIG. 19 shows the ³¹P{¹H} solid state NMR of [1-propene][BAr^{F}₄] complex, measured at room temperature.

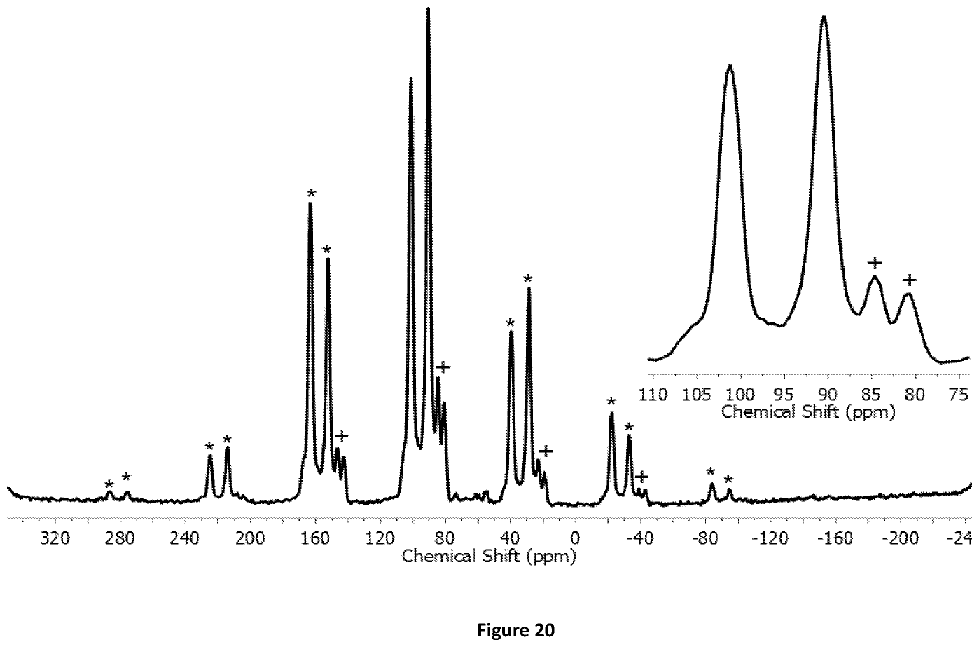

FIG. 20 shows the ³¹ P{¹H} solid state NMR of [1-propene][BAr^{F}₄] complex, measured at 158 K.

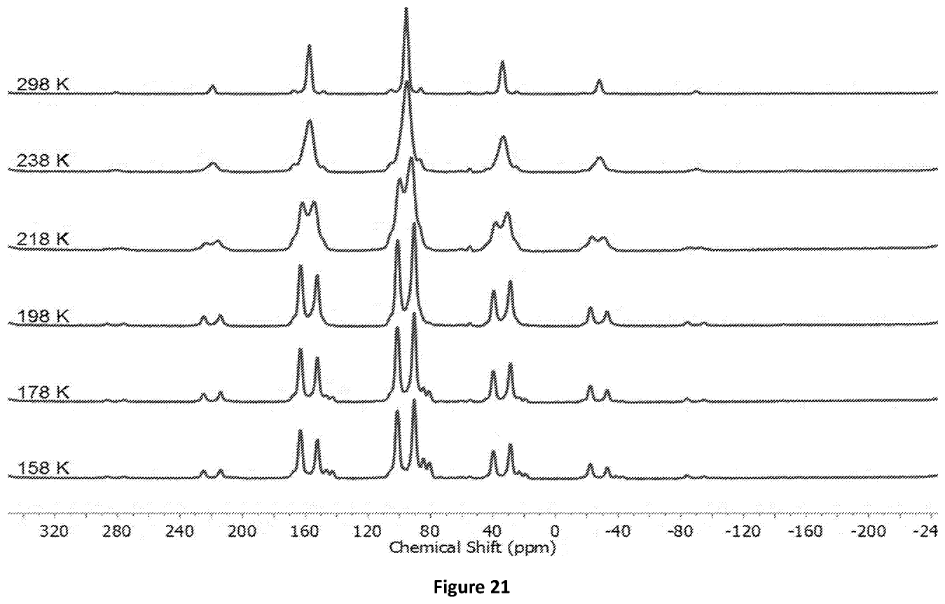

FIG. 21 shows a stack plot of the variable temperature solid-state ³¹ P{¹H} NMR, demonstrating the coalescence of central resonance.

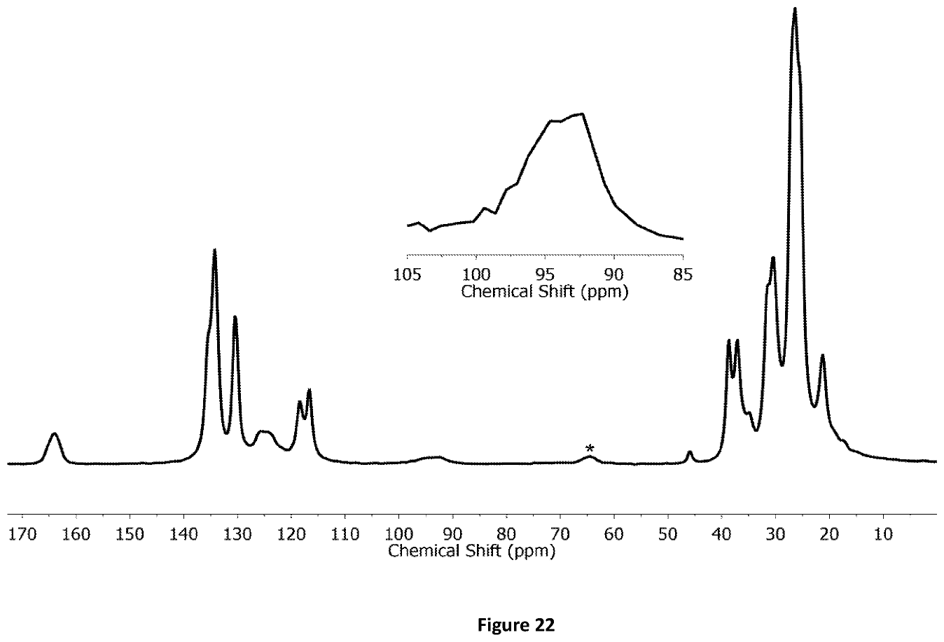

FIG. 22 shows the ¹³C{¹H} solid state NMR spectrum of [1-propene][BAr^{F}₄], measured at room temperature.

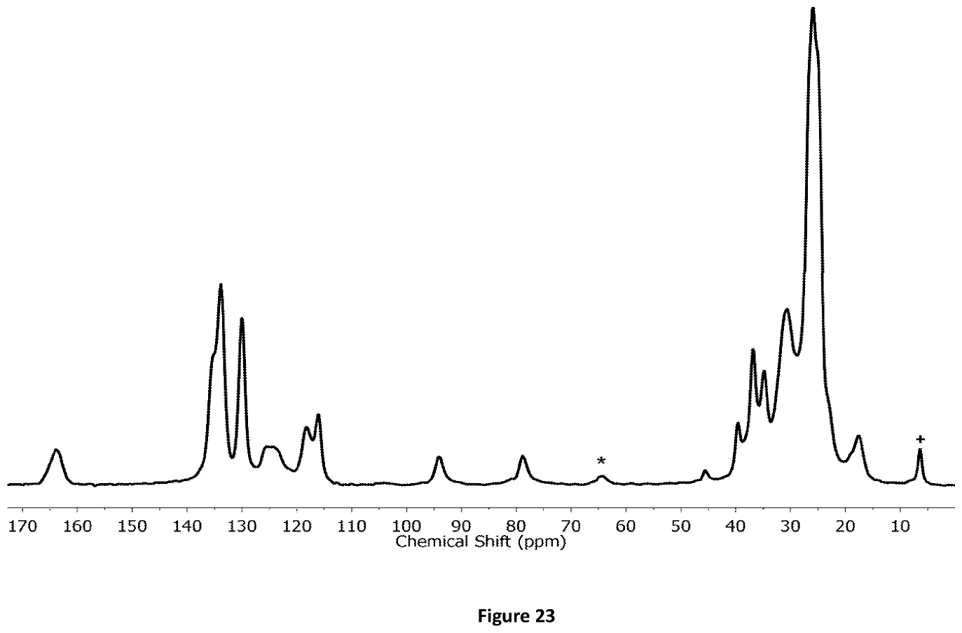

FIG. 23 shows the ¹³C{¹H} solid state NMR spectrum of [1-propene][BAr^{F}₄], measured at 158 K.

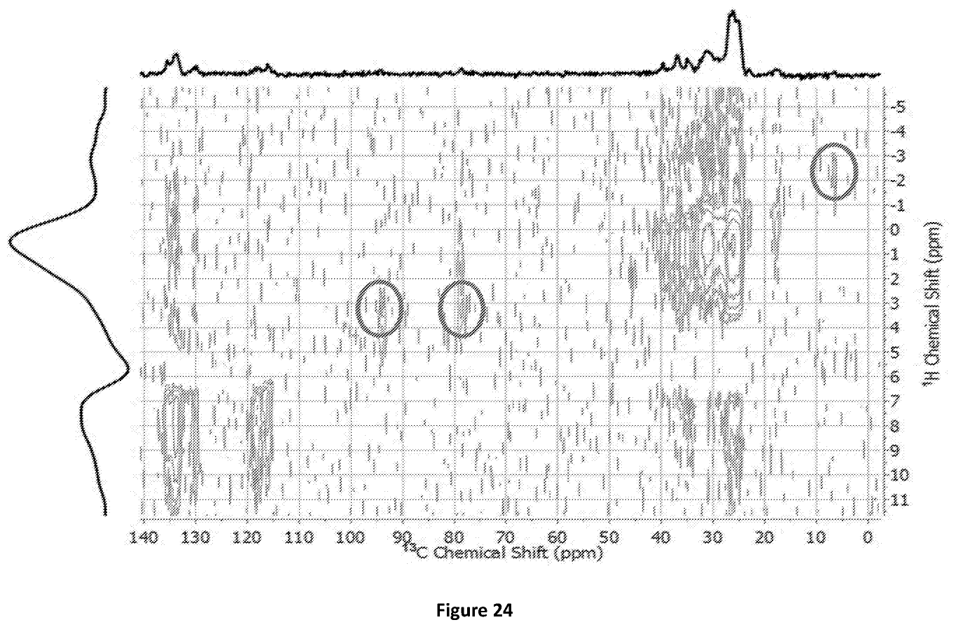

FIG. 24 shows the solid-state fslg-HETCOR 13C/1H spectrum of [1-propene][BAr^{F}₄] the propene complex, measured at 158 K.

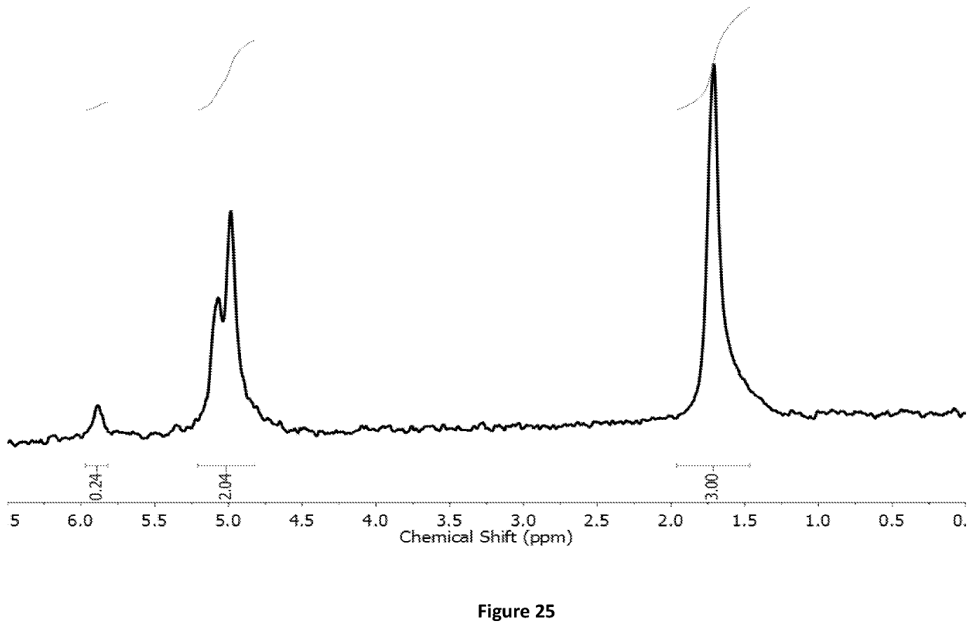

FIG. 25 shows the gas phase ²H{¹H} NMR of the headspace of the reaction of [1-NBA][BAr^{F}₄] with propene-D₃ after 1 hour.

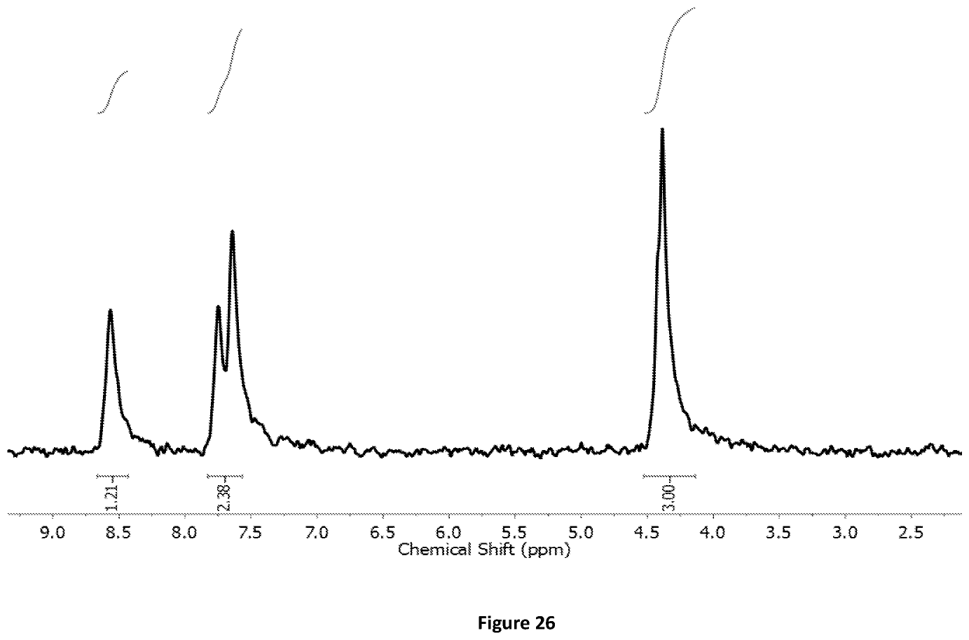

FIG. 26 shows the gas phase ²H{¹H} NMR of the headspace of the reaction of [1-NBA][BAr^{F}₄] with propene-D₃ after 16 hour.

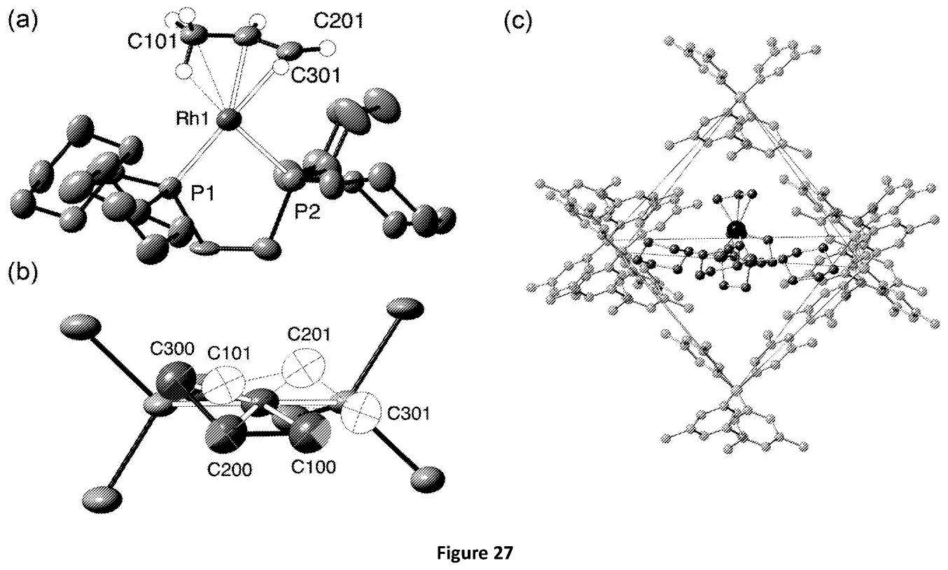

FIG. 27 shows the solidstate structure of [1propene][BAr^{F}₄]. Displacement ellipsoids are shown at the 30% probability level. (a) Cation with selected hydrogen atoms shown; (b) Disordered propene ligand (with the two components shown in red and white); (c) Packing of the [BAr^{F}₄] anions with fluorine atoms omitted for clarity.

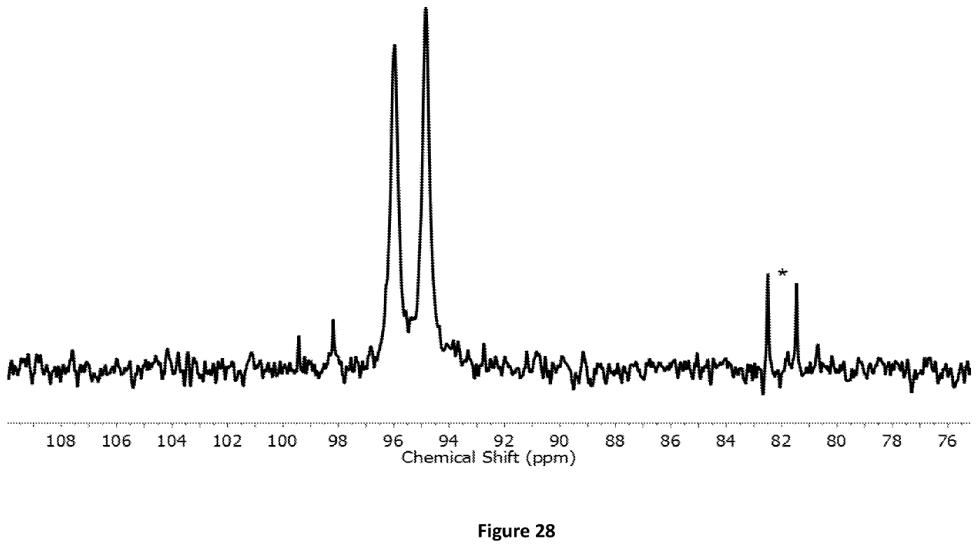

FIG. 28 shows the solution ³¹ P{¹H} NMR spectrum of [1-butene][BAr^{F}₄] after addition of butane to [1-NBA][BAr^{F}₄] in the solid-state,

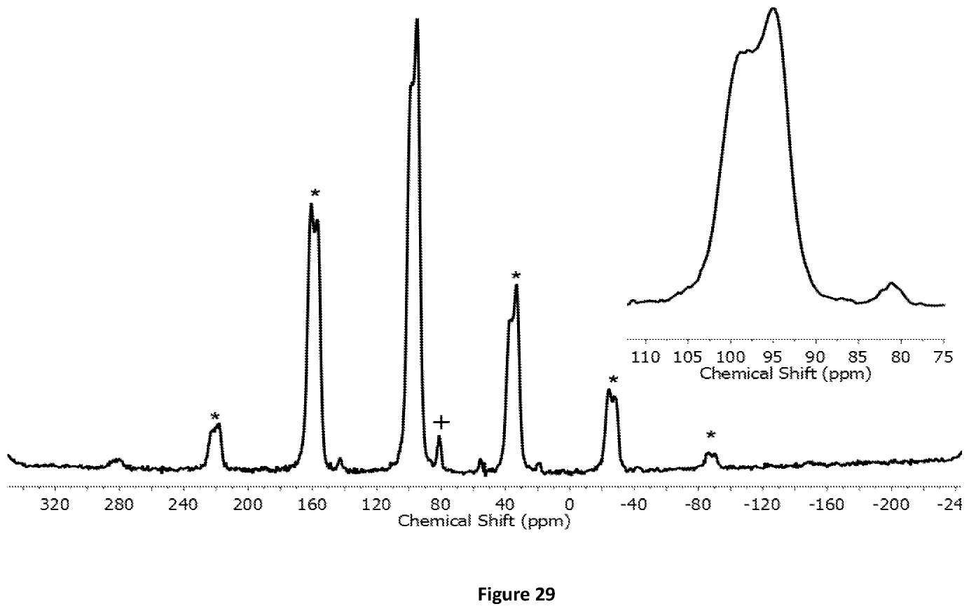

FIG. 29 shows the ³¹P{¹H} solid state NMR spectrum of [1-butene][BAr^{F}₄], 40 minutes after addition of butane to [1-NBA][BAr^{F}₄] at room temperature direct in the NMR rotor.

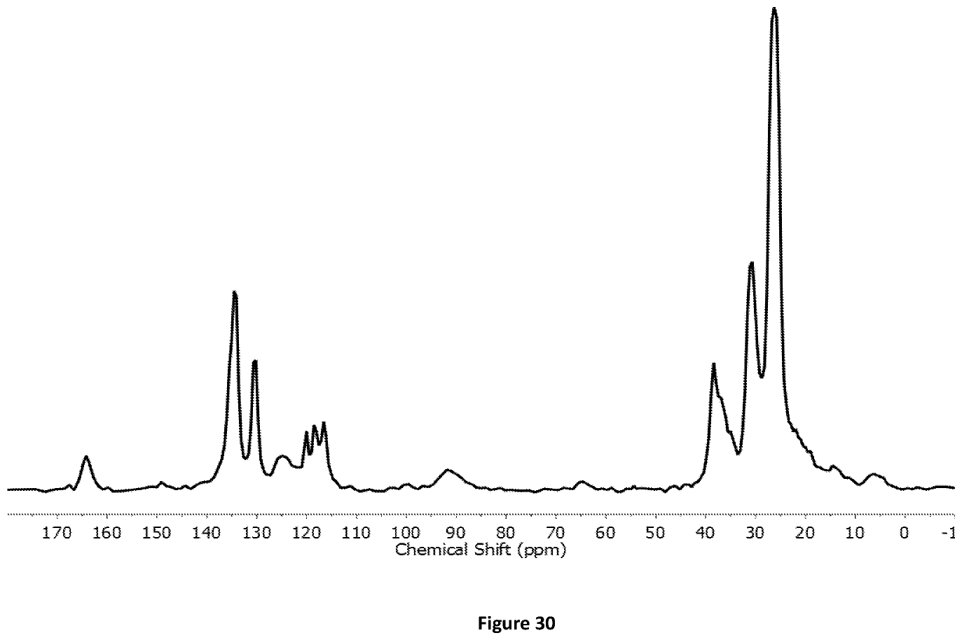

FIG. 30 shows the solid state ¹³C{¹H} NMR spectrum of [1-butene][BAr^{F}₄], 40 minutes after addition of butane to [1-NBA][BAr^{F}₄] at room temperature direct in the NMR rotor.

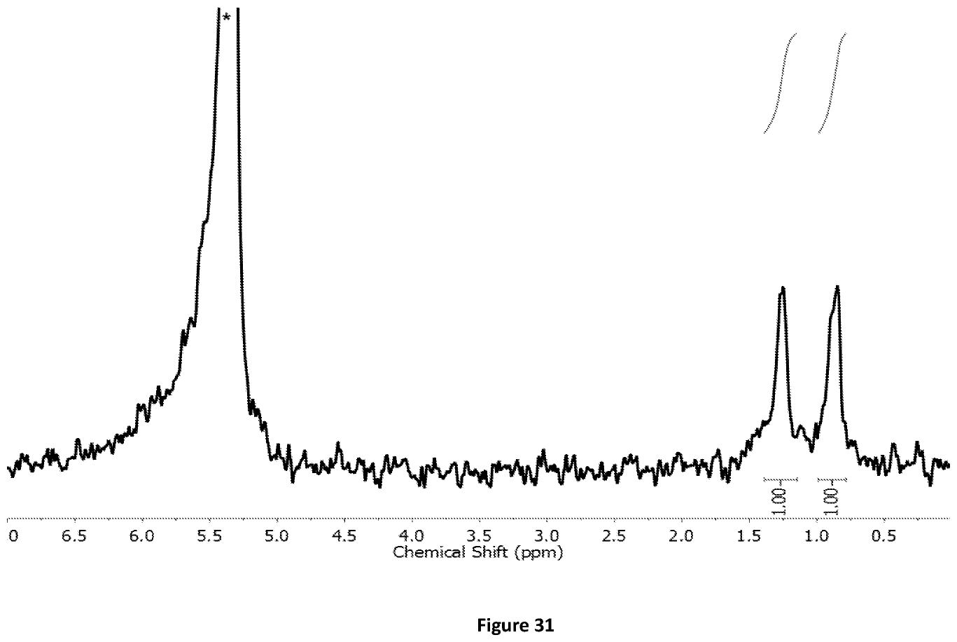

FIG. 31 shows the solution ²H{¹H} NMR of the product of D₂ addition to the in-situ formed [1-butene][BAr^{F}₄] complex.

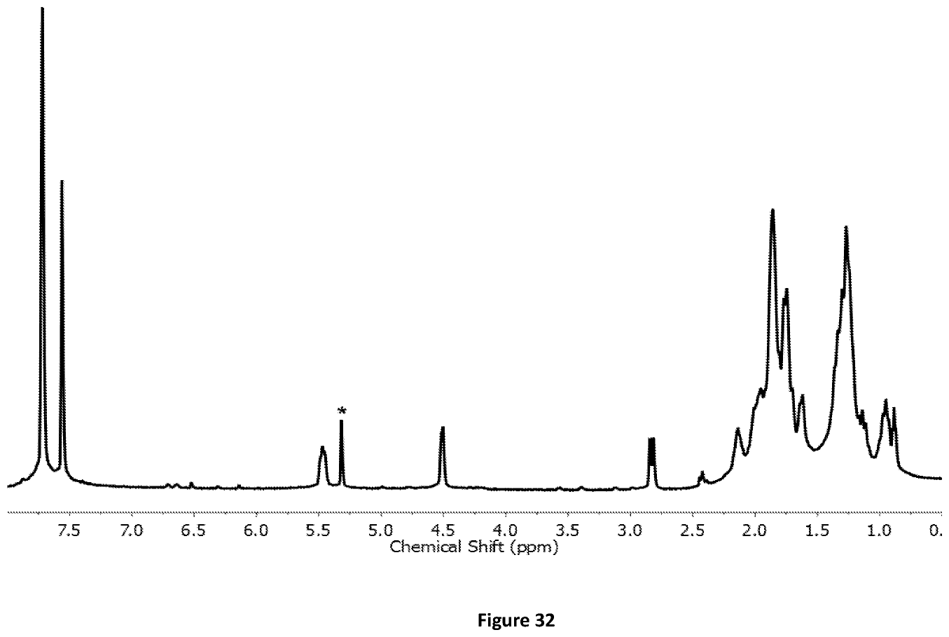

FIG. 32 shows the solution phase 1H NMR spectrum of [1butadiene][BAr^{F}₄] (CD2Cl2, measured at 298 K).

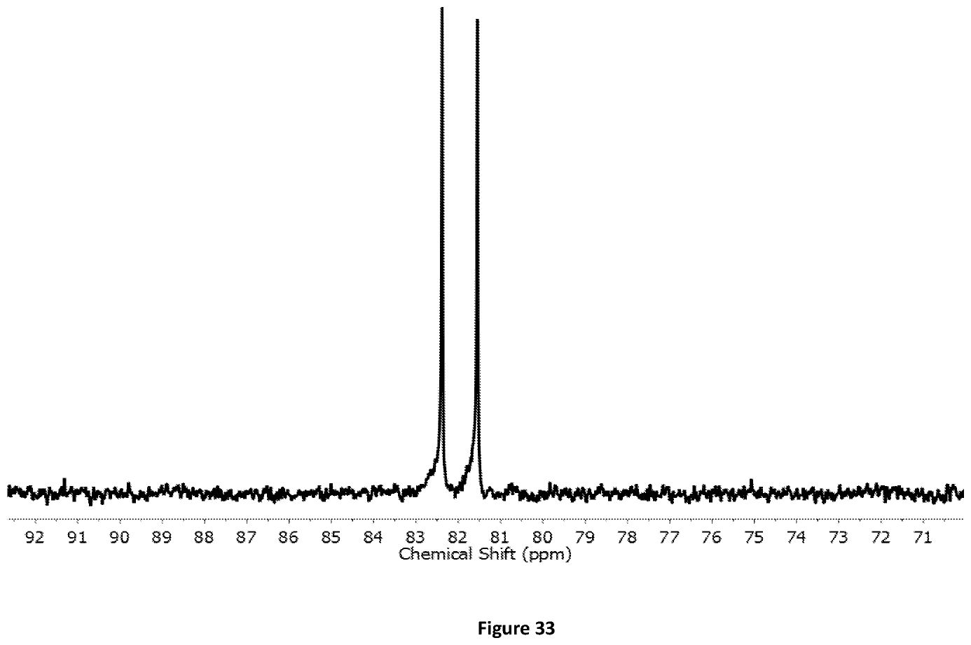

FIG. 33 shows the solution phase 31P{1H} NMR spectrum of [1butadiene][BAr^{F}₄] measured at 298 K.

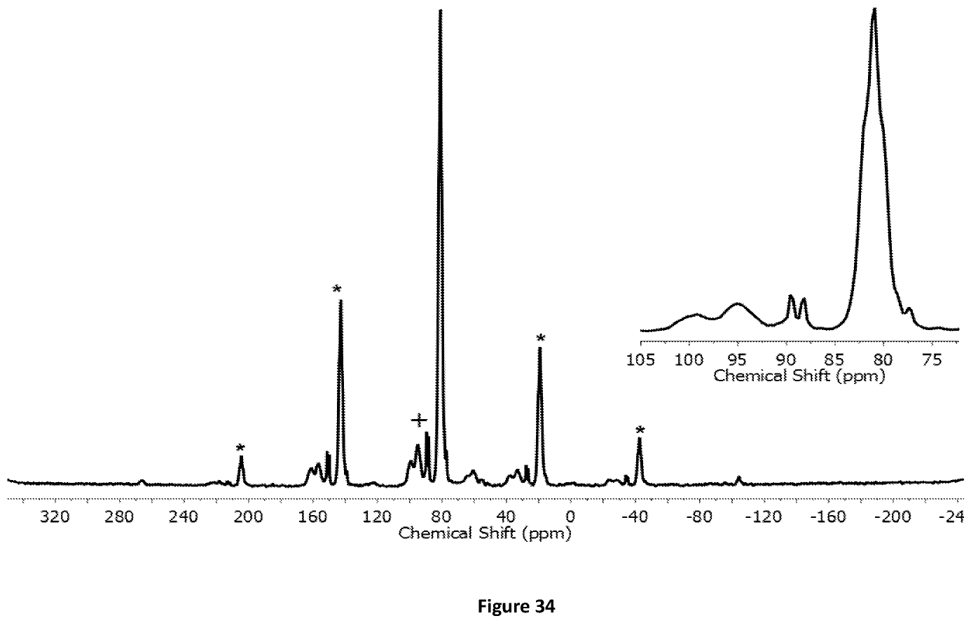

FIG. 34 shows the solid state 31P{1H} NMR spectrum of [1butadiene][BAr^{F}₄] formed after 6 hours addition of 1-butene to [1-NBA][BAr^{F}₄].

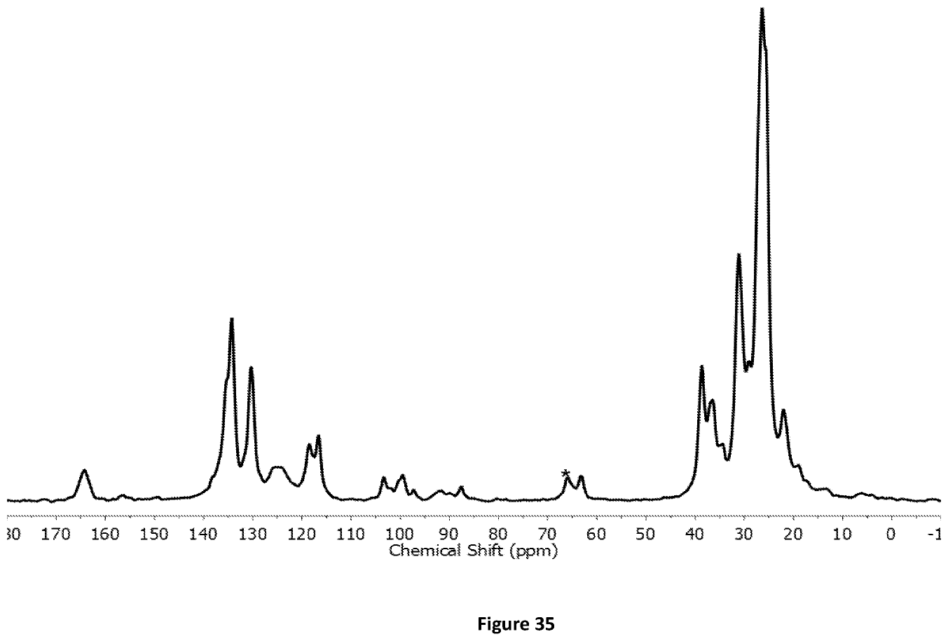

FIG. 35 shows the 13C{1 H} NMR solid state spectrum of [1-butadiene][BAr^{F}₄].

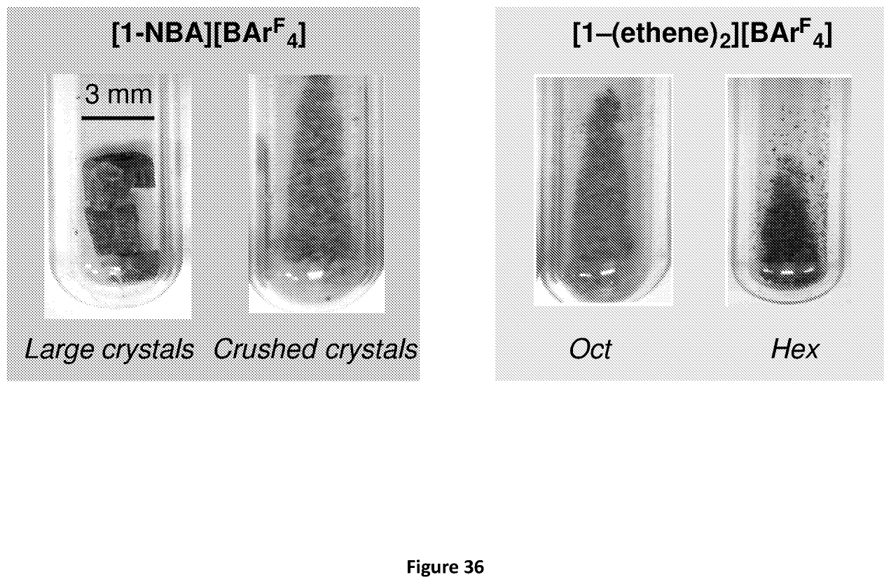

FIG. 36 shows the physical forms of [1-NBA][BAr^{F}₄] (big crystals ca, 1×1×2 mm), [1-NBA][BAr^{F}₄] (crushed crystals ca. 0.1×0.1×0.1 mm), [1-(ethene)₂][BAr^{F}₄]-Oct (crushed crystals ca. 0.1×0.1×0.1 mm) and [1-(ethene)₂][BAr^{F}₄]-Hex (crushed crystals ca. 0.1×0.1×0.1 mm) used for the gas/solid isomerization of 1-butene to trans and cis 2-butane.

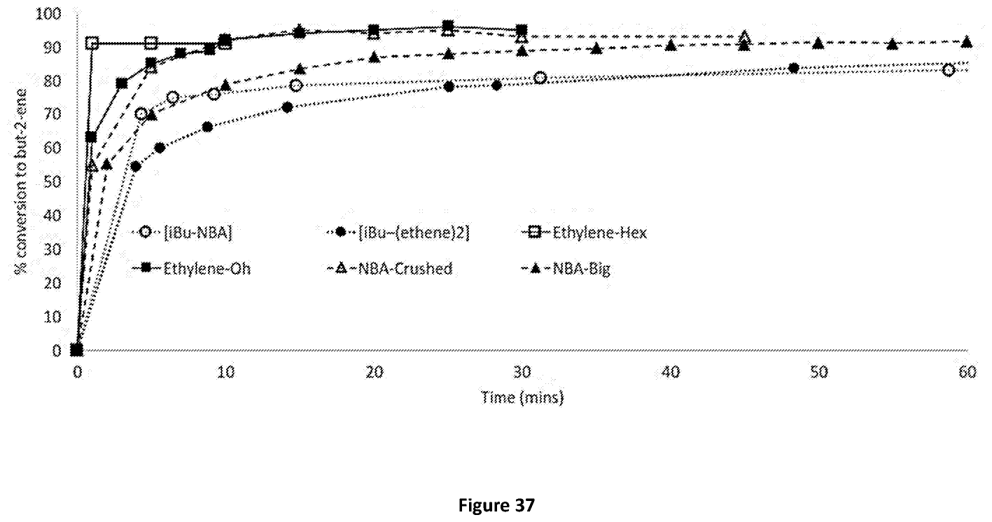

FIG. 37 shows a comparison of [1-NBA][BAr^{F}₄] (big crystals), [1-NBA][BAr^{F}₄] (crushed crystals), [1-(ethene)₂][BAr^{F}₄]-Oct (crushed crystals) and [1-(ethene)₂][BAr^{F}₄]-Hex (crushed crystals) in the isomerization of 1-butene to 2-butene as measured by gas phase NMR spectroscopy. ['Bu-NBA] and ['Bu-(ethene)₂] are comparative examples. All catalysts=˜3 mg sample (˜2 μmol), except [1-(ethene)₂][BAr^{F}₄]-Hex=6 mg sample (4 μmol). 1-butene=15 psi (83 μmol at 298 K).

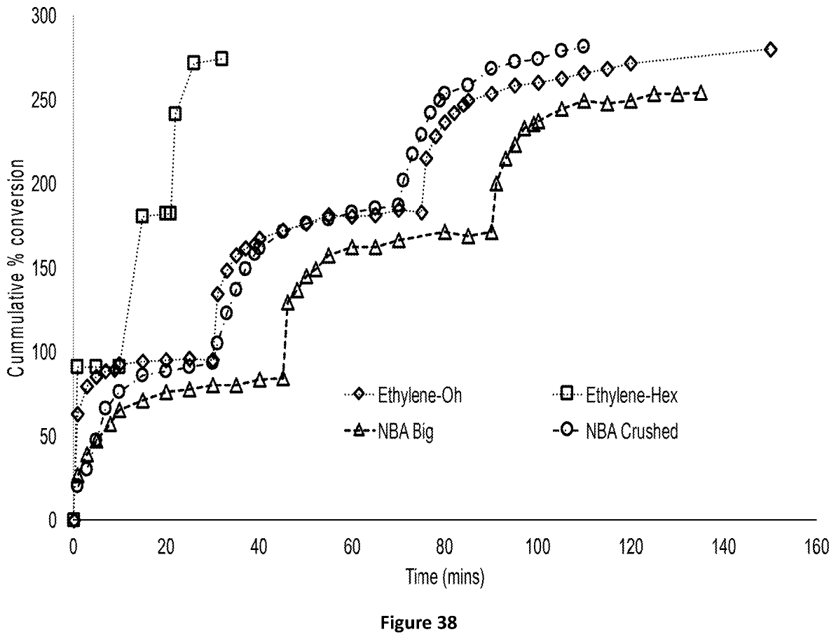

FIG. 38 shows a comparison of recycling of [1-NBA][BAr^{F}₄] (big crystals), [1-NBA][BAr^{F}₄] (crushed crystals), [1-(ethene)₂][BAr^{F}₄]-Oct (crushed crystals) and [1-(ethene)₂][BAr^{F}₄]-Hex (crushed crystals) in the isomerization of 1-butene to 2-butene as measured by gas phase NMR spectroscopy. Conditions as FIG. 37. Lines are drawn to guide the eye.

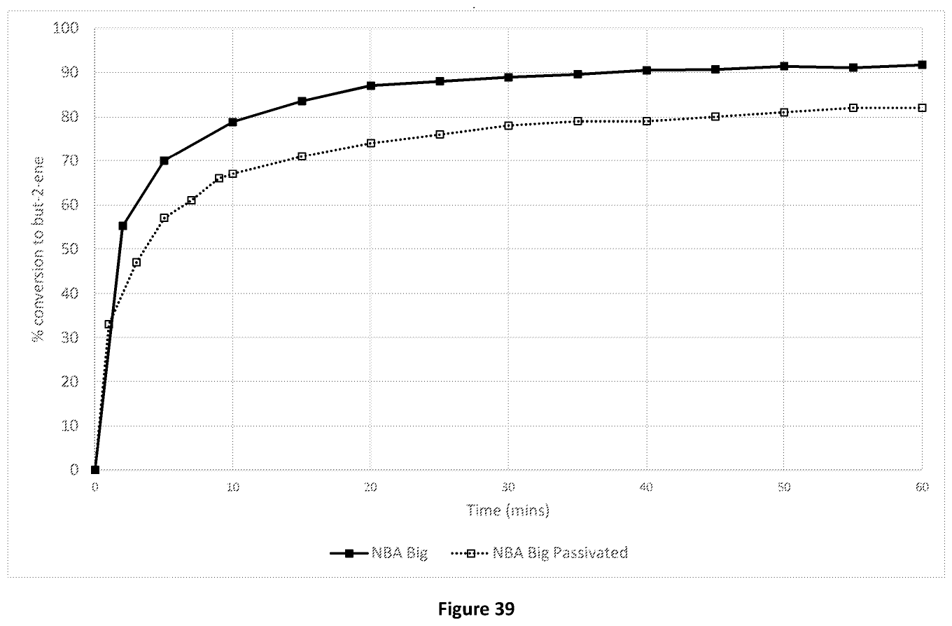

FIG. 39 shows time/conversion behaviour for [1-NBA][BAr^{F}₄] (big crystals) and CO-passivated [1-NBA][BAr^{F}₄] (big crystals) in the conversion of 1-butene to 2-butene.

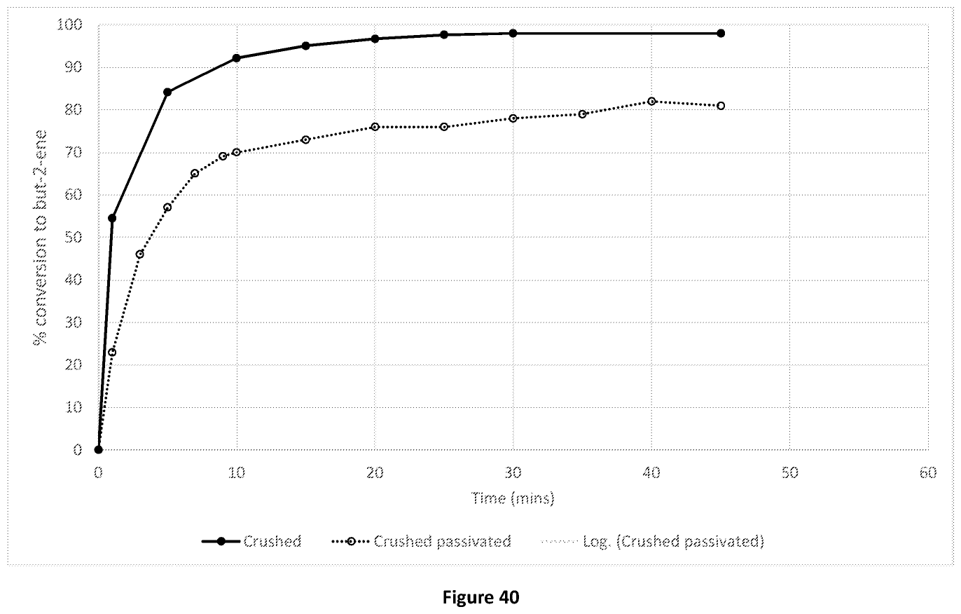

FIG. 40 shows time/conversion behaviour for [1-NBA][BAr^{F}₄] (crushed crystals) and CO-passivated [1-NBA][BAr^{F}₄] (crushed crystals) in the conversion of 1-butene to 2-butene.

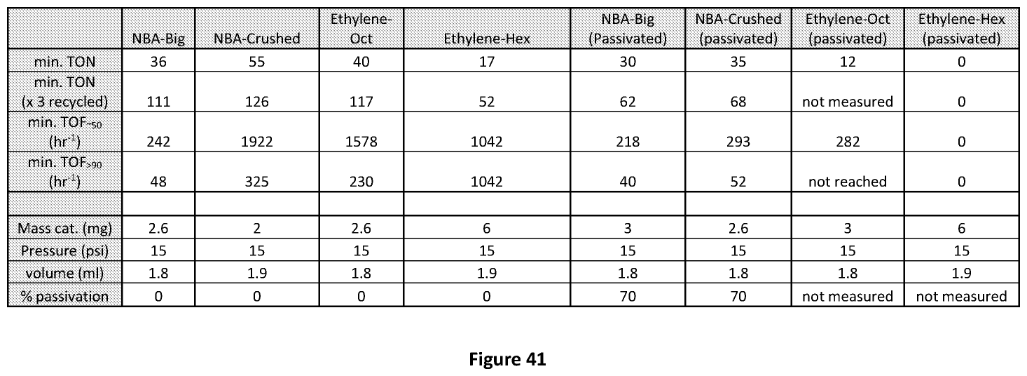

FIG. 41 provides an overview of the catalytic properties of the various exemplary catalysts.

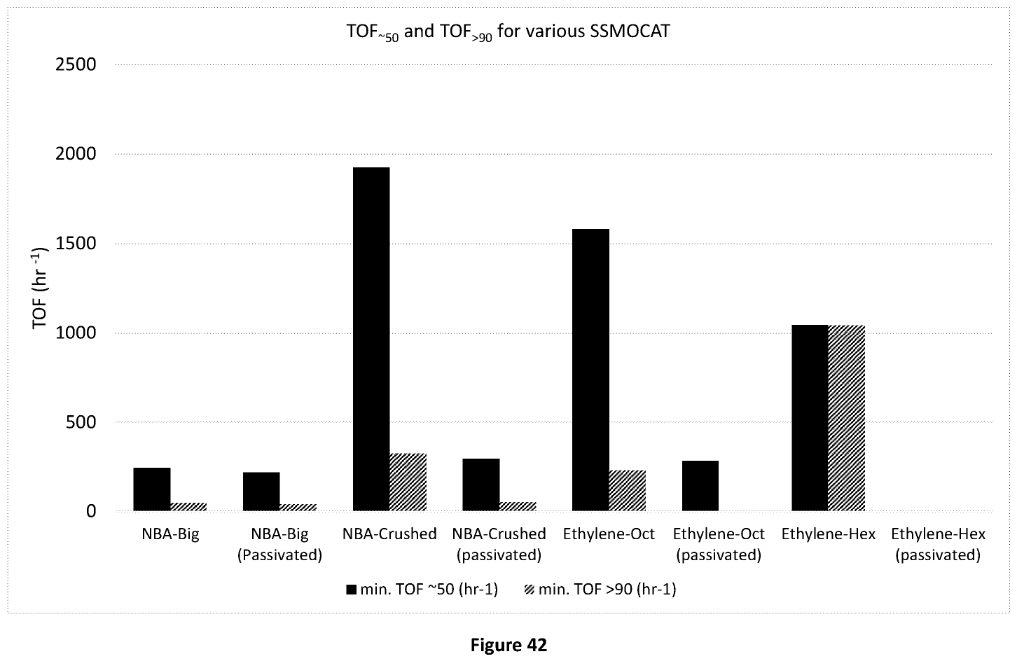

FIG. 42 provides an overview of the TOF_{˜50} and TOF_{>90} for the various exemplary catalysts.

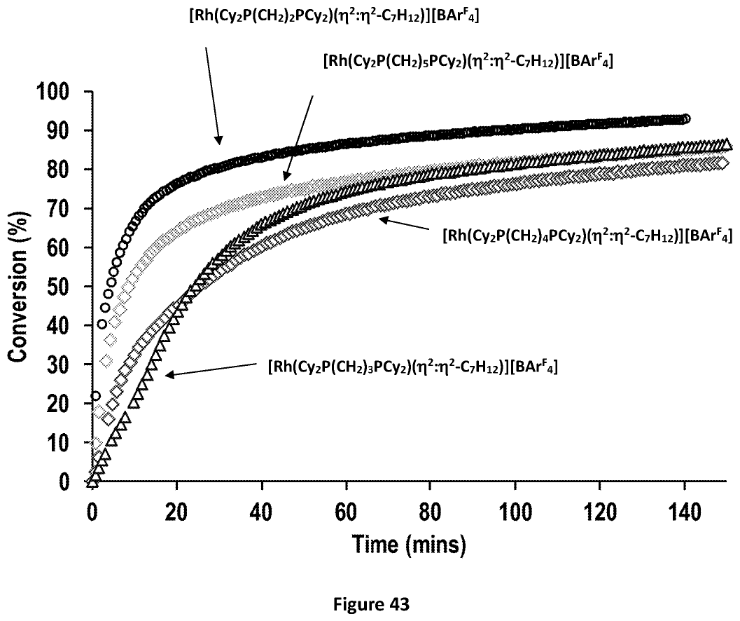

FIG. 43 shows data for the catalytic isomerization of but-1-ene to but-2-ene by crystals off [Rh(Cy₂P(CH₂)₂PCy₂)(η²:η²-C₇H₁₂)][BAr^{F}₄] (circles), [Rh(Cy₂P(CH₂)₅PCy₂)(η²:η²-C₇H₁₂)][BAr^{F}₄] [Rh(Cy₂P(CH₂)₄PCY₂)(η²:η²-C₇H₁₂)_{][BAr}^{F}₄] (rhomboids), (squares) and [Rh(Cy₂P(CH₂)₃PCY₂)(η²:η²-C₇H₁₂)][BAr^{F}₄] (triangles), demonstrating the influence of the phosphine linker. The conversion was measured by gas phase ¹H NMR spectroscopy comparing the integrals corresponding to 1-butene and 2-butene.

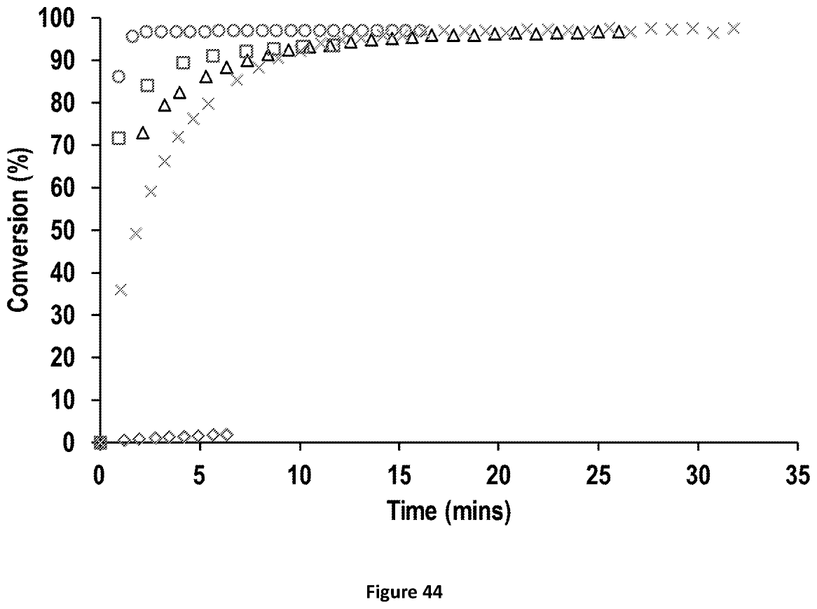

FIG. 44 shows data for the catalytic isomerization of but-1-ene to but-2-ene by crushed [Rh(Cy₂P(CH₂)₂PCy₂)(η²:η²-C₇H₁₂)][BAr^{F}₄] (circles), [Rh(Cy₂P(CH₂)₂PCy₂)(H)₂][Al{OC(CF₃)₃}₄] (squares), [Rh(Cy₂P(CH₂)₂PCy₂)(η²:η²-C₇H₁₂)][BAr^{c1}₄] (triangles), [Rh(Cy₂P(CH₂)₂PCy₂)(η²:η²-C₇H₁₂)][BAr(F)₄] (crosses) and [Rh(Cy₂P(CH₂)₂PCy₂)(η²:η²-C₇H₁₂)][BAr^{H}₄] (rhomboids), demonstrating the influence of cation. The conversion was measured by gas phase ¹H NMR spectroscopy comparing the integrals corresponding to 1-butene and 2-butene.

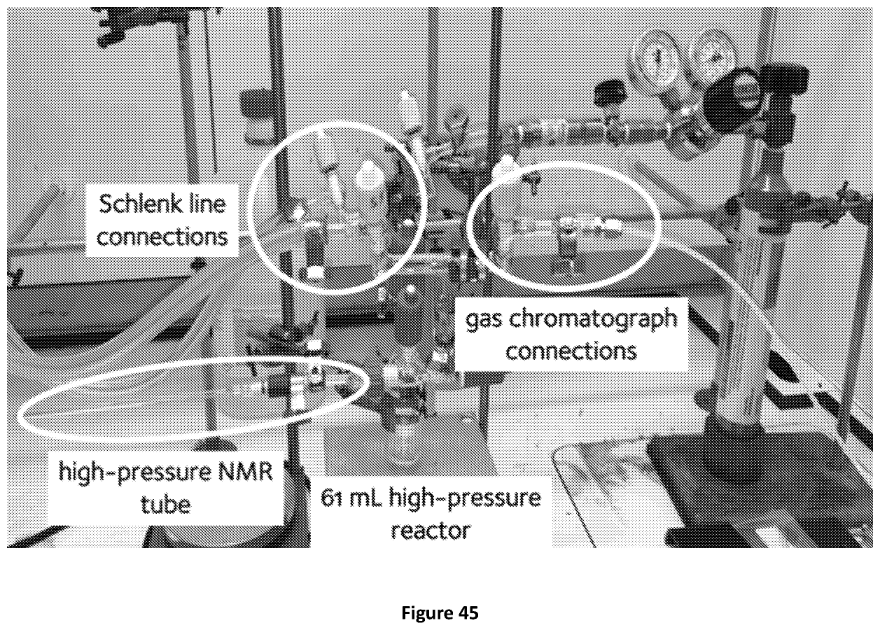

FIG. 45 shows the experimental set-up to study the gas-phase isomerization of 1-butene to 2-butene.

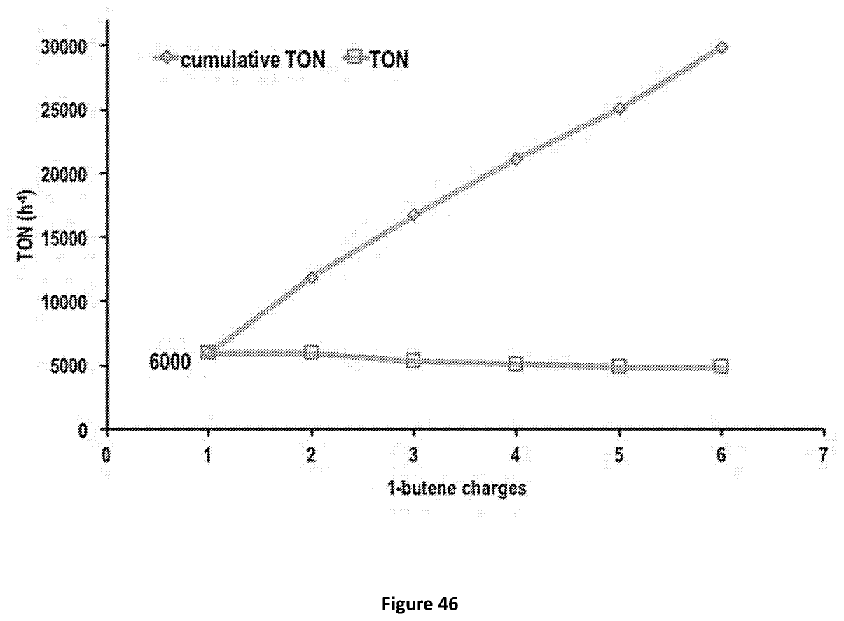

FIG. 46 shows catalytic data for the isomerization of 1-butene to 2-butene using a batch reactor of 61 mL of volume for recharges of 1-butene. Catalysts [Rh(Cy₂P(CH₂)₂PCy₂)(η²:η²-ethene)₂][BAr^{F}₄]-Hex (1 mg) and subsequent recharges.

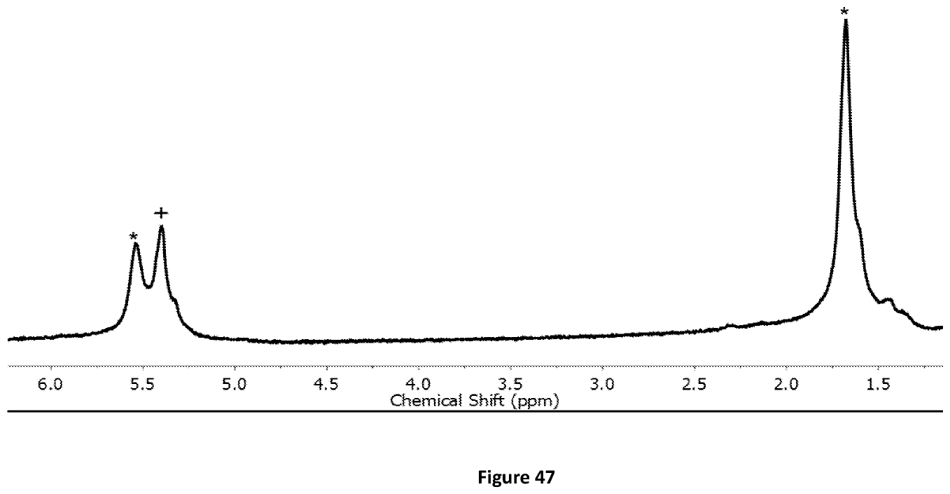

FIG. 47 shows a gas-phase NMR of [1-NBA][BAr^{F}₄]-crushed left under ethene for two weeks.

### MATERIALS AND METHODS

All manipulations (unless otherwise stated) were performed under an atmosphere of argon, using standard Schlenk techniques on a dual vacuum/inlet manifold or by employment of an MBraun glovebox. Glassware was dried in an oven at 130° C. overnight prior to use. Pentane, hexane and CH₂Cl₂ were dried using an MBraun SPS-800 solvent purification system and degassed by three freeze-pump-thaw cycles. CD₂Cl₂ and C₆H₅F were both dried by stirring over CaH₂ overnight before being vacuum distilled and subsequently degassed by three freeze-pump-thaw cycles. 1,2-F₂C₆H₄ was stirred over Al₂O₃ for two hours then over CaH₂ overnight overnight before being vacuum distilled and subsequently degassed by three freeze-pump-thaw cycles. Ethylene, propylene and but-1-ene were all supplied by CK gases. Propylene-d₃ was supplied by Cambridge isotopes laboratory.

Solution NMR data were collected on either a Brucker AVD 500 MHz or a Bruker Ascend 400 MHz spectrometer at room temperature unless otherwise started. Non-deuterated solvents were locked to standard CD₂Cl₂ solutions. Residual protio solvent resonances were used as a reference for ¹H NMR spectra. A small amount of CD₂Cl₂ was added as a reference for ²H{¹H} NMR spectra. ³¹P{H} NMR spectra were referenced externally to 85% H₃PO₄. All chemical shifts (δ) are quoted in ppm and coupling constants in Hz.

¹H/¹³C solid state NMR (SSNMR) spectra (including two dimensional measurements) were obtained on a Bruker Avance III HD spectrometer equipped with a 9.4 Tesla magnet, operating at 399.9 MHz for ¹H and 100.6 MHz for ¹³C using 4 mm O.D. rotors containing approximately 70 mg of sample and a MAS rate of 10 kHz. Powdered microcrystalline samples were prepared by grinding using the back of a spatula in a glovebox and subsequently loaded into 4 mm rotors. Hydrogenation and deuteration reactions were undertaken by exposing the open rotors in a J. Young's flask to an atmosphere (2 atm) of H₂/D₂ respectively before removal of the atmosphere and capping the rotors in a glovebox. For ¹³C CP/MAS a sequence with a variable X-amplitude spin-lock pulse¹ and spinal64 proton decoupling was used. 4500 transients were acquired using a contact time of 2.5 ms, an acquisition time of 25 ms (2048 data points zero filled to 32 K) and a recycle delay of 2 s. All ¹³C spectra were referenced to adamantane (the upfield methine resonance was taken to be at δ=29.5 ppm² on a scale where δ(TMS)=0 ppm as a secondary reference. For the fslg-HETCOR,³ 128 transients (2048 data points in F2) and 80 increments in F1 (zero filled to 4k×1k) were acquired with a contact time 0.4 ms and a recycle delay of 5 s. ³¹ P{¹H} spectra were reference externally to 85% H₃PO₄. Low temperature measurements were undertaken using standard Bruker variable temperature set-up.

Gas phase ¹H NMR spectroscopy was carried out using a Bruker Ascend 400 MHz spectrometer. The T1 delay was set to 1 s, and this has been previously shown to allow for the accurate comparison of integrals. Samples were loaded into a high-pressure NMR tube sealed with a Teflon stopcock, before being transferred to a Schlenk vacuum line, evacuated and then loaded with the gaseous reagents (via a custom made glass T-piece adaptor). The spectrometer was locked and shimmed to a separate CD₂Cl₂ sample in a similar bore tube, the sample was then replaced and spectra run. For isomerisation catalytic runs the machine was locked and shimmed before the gaseous reagents were added (full experimental details of isomerization catalysis are below).

Electrospray ionisation mass spectrometry (ESI-MS) was carried out using a Bruker MicrOTOF instrument directly connected to a modified Innovative Technology glovebox.⁴ Typical acquisition parameters were used (sample flow rate: 4 μL min⁻¹, nebuliser gas pressure: 0.4 bar, drying gas: Argon at 333 K flowing at 4 L min⁻¹, capillary voltage: 4.5 kV, exit voltage: 60 V). The spectrometer was calibrated using a mixture of tetraalkyl ammonium bromides [N(CₙH₂ₙ₊₁)₄]Br (n=2-8, 12, 16 and 18). Samples were diluted to a concentration of 1×10⁻⁶ M in the appropriate solvent before sampling by ESI-MS.

Single crystal X-ray diffraction data for all samples were collected as follows: a typical crystal was mounted on a MiTeGen Micromounts using perfluoropolyether oil and cooled rapidly to 150 K in a stream of nitrogen gas using an Oxford Cryosystems Cryostream unit.⁵ Data were collected with an Agilent SuperNova diffractometer (Cu Kα radiation, λ=1.54180 Å). Raw frame data were reduced using CrysAlisPro.^{6,7} The structures were solved using SuperFlip⁸ and refined using full-matrix least squares refinement on all F² data using the CRYSTALS program suite.^{9, 10} In general distances and angles were calculated using the full covariance matrix. Dihedral angles were calculated using PLATON.¹¹

Isomerisation runs were carried out in the gas phase by loading a high pressure NMR tube (of known volume) with a crystalline sample of the catalyst in an argon-filled glovebox. The tubes were sealed by a Teflon stopcock and transferred to a Schlenk line fitted with a custom-built glass T-piece adaptor, allowing for exposure to vacuum/argon on one side, and the reagent gas on the other side. The T-piece and connecting tubing were thrice pumped and refilled with argon, then thrice pumped and refilled with but-1-ene, before being evacuated (<1×10⁻² mbar) and subsequently opening the Teflon stopcock on the NMR tube (thus exposing the argon-filled tube to dynamic vacuum). During this final evacuation the NMR machine was prepared by locking and shimming to a sample of CD₂Cl₂ in a similar bore NMR tube. The sample was then refilled with but-1-ene gas as a timer was simultaneously started. The tube was sealed and transferred to the NMR machine as quickly as possible. The first data collection was immediately started. The extent of conversion was measured by the comparison of the integral of the two alkene resonances of but-2-ene and the alkyl CH₂ resonance of but-1-ene. These have been previously shown to be comparable by gas phase NMR. TON and TOF are calculated assuming that all site are equally catalytically active, and are therefore, a minimum number. Intuitively surface sites would be more active than those at the centre of the bulk by a simple mass transit argument.

### Example 1

### Synthesis and Characterisation of Rh Complexes

### 1.1—Synthesis and characterisation of [(Cy₂PCH₂CH₂PCy₂)Rh(η²:η²-C₇H₁₂)][BAr^{F}₄] ([1-NBA][BAr^{F}₄])

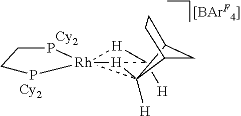

One Schlenk flask was charged with [Rh(COD)₂][BAr^{F}₄] (500 mg, 0.423 mmol) and another was filled with Cy₂PCH₂CH₂PCy₂ (180 mg, 0.426 mmol). Both solids were dissolved in CH₂Cl₂ (30 ml each) and the phosphine was added to [Rh(COD)₂][BAr^{F}₄] with vigorous stirring. The solution was allowed to stir for one hour before the solvent was removed in vacuo. The subsequent solid was washed with pentane (3×20 ml) before being taken up in C₆H₅F (30 ml) and filtered via cannula into a Young's flask. The solution was freeze-pump-thaw degassed three times then H₂ gas was added (1 bar). The solution was allowed to stir for four hours before the H₂ and solvent was removed in vacuo. The remaining solidwas washed with pentane (3×20m1) then taken up in CH₂Cl₂ (50 ml) and filtered via cannula into a Schlenk flask. This solution was stirred vigorously and an excess of norbornadiene was added (0.6 ml, 5.904 mmol) and the solution darkened over 15 minutes to a blood-orange red. The solvent was removed in vacuo and excess norbornadiene and C₆H₅F were removed by washing with pentane (3×20 ml) before the resultant solid was taken up in the minimum volume of CH₂Cl₂ and filtered into a Young's crystallization tube and layered with pentane. Yield 370 mg (59%). [Rh(Cy₂PCH₂CH₂PCy₂)(η²:η²-C₇H₈)][BAr^{F}₄]. Hydrogenation (1 atm) of a crystalline sample of [Rh(Cy₂PCH₂CH₂PCy₂)(η²:η²-C₇H₈)][BAr^{F}₄] led to the quantitative formation of [1-NBA][BAr^{F}₄] after five minutes. The crystalline sample goes opaque but there is little other colour change.

³¹P{¹H} SS-NMR (162 MHz, 10 kHz spin rate): δ 110.5 (two overlapping d, J_{Rh-P1}=207 Hz, J_{Rh-P2}=216 Hz). ¹³C{¹H} SS-NMR (101 MHz, 10 kHz spin rate): δ 163.18 (br, BAr^{F}₄), 134.54 (br, BAr^{F}₄), 129.80 (br, BAr^{F}₄), 124.30 (br, BAr^{F}₄), 118.19 (br, BAr^{F}₄), 115.84 (br, BAr^{F}₄), 43.71, 39.65, 38.98, 35.95, 35.34, 31.86, 31.20, 30.17, 29.01, 26.90, 25.33, 20.69 (multiple aliphatic resonances). ¹H projection from ¹H/¹³C Frequency Switched LeeGoldburg HECTOR SS-NMR: δ 8.09 (sh), 7.10 (m, br), 0.83 (s), −1.82 (w). ¹³C projection from ¹H/¹³C Frequency Switched LeeGoldburg HECTOR SS-NMR: δ 134.80, 130.00, 118.60, 116.00, 44.10, 39.50, 36.00, 30.70, 27.40, 25.50, 21.40. Elemental analysis found (calculated): C 52.46 (52.55) H 4.80 (4.89)

### 1.2—Synthesis and characterisation of [Rh(Cy₂PCH₂CH₂PCy₂)(η²C₂H₄)₂][BAr^{F}₄] ([1-(ethene)₂][BAr^{F}₄])

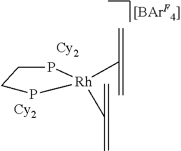

### Attempted Solution Phase Synthesis

A crystalline sample of [(Cy₂PCH₂CH₂FCy₂)Rh(η⁶-F₂C₆H₄)][BAr^{F}₄] ([¹⁻C₆H₄F₂][BAr_{F4}]¹³) (25 mg, 0.0166 mmol) was taken up in CD₂Cl₂ (0.5 ml) in a high pressure NMR tube. This was freeze-pump-thaw degassed (<1×10⁻² mbar) three times before ethylene gas (1 bar) was added. An immediate darkening of the yellow solution to orange occurred. ³¹P{¹H} NMR spectroscopy indicated that near quantitative conversion to [1-(ethene)₂][BAr^{F}₄] had occurred after 15 minutes (FIG. 1, bottom), however an amount of the starting [1-C₆H₄F₄][BAr^{F}₄] remains (labelled *), indicating it is in equilibrium with [1-(ethene)₂][BAr^{F}₄]. Any attempted work-up involving a vacuum results in the complete decomposition of the species, to presumed solvent (C—H or C—Cl) activated products (indicated from mass spectroscopy showing the presence of chloride-bridged rhodium dimers). Furthermore leaving [1-(ethene)₂][BAr^{F}₄] complex in CH₂Cl₂ solution, at room temperature, also resulted in similar decomposition over a period of approximately an hour. To date it has not been possible to isolate [1-(ethene)₂][BAr^{F}₄] via solution methods.

### Solid state synthesis of octahedral [Rh(Cy₂PCH₂CH₂PCy₂)(η²-C₂H₄)₂][BAr^{F}₄] ([1-(ethene)₂][BAr^{F}₄]-Oct)

To an orange sample of crystalline [1-NBA][BAr^{F}₄] (25 mg, 0.0168 mmol) in an evacuated (<1×10⁻² mbar) J Young's flask (c. 50 ml) ethylene gas (1 bar, 298 K) is added and left standing overnight. Little colour change is observed, though the crystals take on the appearance of liquid on the surface assumed to be norbornane. It is not possible to remove the residual norbornane (attempts to do so by washing with pentane did not work), however the synthesis goes in >95% yield by ³¹P{¹H} solid state NMR spectroscopy and ³¹P{¹H} solution NMR spectroscopy when dissolved up in CD₂Cl₂ (the only other signal being due to an uncharacterised decomposition product, which mass spectroscopic evidence suggests a product of CH₂Cl₂ activation). After 16 hours in CD₂Cl₂ the compound decomposes to a range of products. Dissolving the product in difluorobenzene results in the formation of [1-C₆H₄F₂][BAr^{F}₄].

### Solid state synthesis of hexagonal [Rh(Cy₂PCH₂CH₂PCy₂)(η² -C₂H₄)₂][BAr^{F}₄] ([1-(ethene)₂][BAr^{F}₄]-Hex)

To an orange sample of crystalline [1-NBA][BAr^{F}₄] (100 mg, 67.3 μmol) in an evacuated (<1×10⁻² mbar) J Young's flask (c. 50 ml) ethylene gas (1 bar, 298 K) is added and left standing overnight, to form [1-(ethene)₂][BAr^{F}₄]-Oct. Working under an atmosphere of ethylene (1 bar), the sample is then dissolved in a minimum volume of freshly degassed CH₂Cl₂ before quick filtration via cannula and layering with freshly degassed pentane. The sample is then stored at −78° C. and allowed to crystallise over at least a week. Single crystals, directly selected from the mother liquor, are suitable for X-ray diffraction analysis, however attempts to isolate the bulk sample resulted in the loss of crystallinity. Nevertheless solution NMR data are identical to [1-(ethene)₂][BAr^{F}₄] confirming the loss of long range order is not due to the loss of ethylene. Due to the limited amount of crystalline material obtained solid-state NMR spectroscopy was not undertaken. Isolated yield on the non-crystalline material: 77 mg (53.3 μmol, 79.2%).

### Characterisation data for [1-(ethene)₂][BAr^{F}₄]

FIG. 1 shows the ³¹P{¹H} solution NMR spectrum of the attempted solution phase synthesis of [1-(ethene)₂][BAr^{F}₄]. The bottom spectrum is measured after 5 minutes reaction, the top spectrum is after attempted work up. The primary resonance in the bottom spectrum corresponds to [1-(ethene)₂][BAr^{F}₄] (vide infra). Both spectra were measured at 253 K to ensure sharp resonances.

¹H solution NMR (CD₂Cl₂, 298 K, 400 MHz) δ: 7.72 (8H, s, o-BAr^{F}₄), 7.56 (4H, s, p-BAr^{F}₄), 4.43 (8H, v br, v_{1/2}=94 Hz, ethylene), 2.0-1.0 ppm (multiple overlapping aliphatic resonances). FIG. 2 shows the solution ¹H NMR spectrum (CD₂Cl₂) of [1-(ethene)₂][BAr^{F}₄] (from dissolved [1-(ethene)₂][BAr^{F}₄]-Oct) measured at room temperature. The resonance marked * is due to residual protio solvent. FIG. 3 shows the solution ¹H NMR spectrum (CD₂Cl₂) of [1-(ethene)₂][BAr^{F}₄] (from dissolved [1-(ethene)₂][BAr^{F}₄]-Hex) measured at room temperature. The resonance marked * is due to residual protio solvent. This spectra is identical in all the key features to that in FIG. 2.

³¹P{¹H} solution NMR (CD₂Cl₂, 298 K, 162 MHz) δ: 73.7 (v. br, v^{1/2}≈500 Hz). FIG. 4 shows the solution ³¹P{¹H} NMR spectrum (CD₂Cl₂) of [1-(ethene)₂][BAr^{F}₄] (from dissolved [1-(ethene)₂][BAr^{F}₄]-0ct) measured at room temperature. The resonance at approximately 82 ppm is due to the presumed solvent induced decomposition product. FIG. 5 shows the solution ³¹ P{¹H} NMR spectrum (CD₂Cl₂) of [1-(ethene)₂][BAr^{F}₄] (from dissolved [1-(ethene)₂][BAr^{F}₄]-Hex) measured at room temperature. The resonance at approximately 82 ppm is due to the presumed solvent induced decomposition product.

¹H solution NMR (CD₂Cl₂, 193 K, 400 MHz) δ: 7.71 (8H, s, o-BAr^{F}₄), 7.54 (4H, s, p-BAr^{F}₄), 4.15 (8H, s, ethylene), 2.0-1.0 ppm (multiple overlapping aliphatic resonances). FIG. 6 shows the solution ¹H NMR (CD₂Cl₂) spectrum of [1-(ethene)₂][BAr^{F}₄] (from dissolved 1-(ethene)₂][BAr^{F}₄]-Oct) measured at 193 K. The resonance marked * is due to residual protio-solvent. FIG. 7 shows the solution ¹H NMR (CD₂Cl₂) spectrum of [1-(ethene)₂][BAr^{F}₄] (from dissolved [1-(ethene)₂][BAr^{F}₄]-Hex) measured at 193 K. The resonance marked * is due to residual protio-solvent. This spectrum is identical in all the key features to that in FIG. 6.

³¹P{¹H} solution NMR (CD₂Cl₂, 193 K, 162 MHz) δ: 73.6 (d, J_{RhP}=145 Hz). FIG. 8 shows the solution ³¹P{¹H} NMR (CD₂Cl₂) spectrum of 1-(ethene)₂][BAr^{F}₄] (from dissolved [1-(ethene)₂][BAr^{F}₄]-Oct) measured at 193 K. This is done on the same sample as FIG. 4, and the solvent induced decomposition product is still present at 82 ppm. FIG. 9 shows the solution ³¹P{¹H} NMR (CD₂Cl₂) spectrum of [1-(ethene)₂][BAr^{F}₄] (from dissolved [1-(ethene)₂][BAr^{F}₄]-Hex) measured at 193 K. This spectrum is effectively indentcal to that in FIG. 8.

³¹P{¹H} solid state NMR (for [1-(ethene)₂][BAr^{F}₄]-Oct; 162 MHz, 10 kHz spin rate) δ: 73.7 (br, v^{1/2}≈410 Hz). FIG. 10 shows the solid state ³¹P{¹H} NMR of [1-(ethene)₂][BAr^{F}₄]-Oct, made by the exposure of [1-NBA][BAr^{F}₄] to ethylene (1 bar), in a solid state NMR rotor, for 2 hours. The inset is a zoom of the central resonance. Resonances marked+are spinning sidebands, those marked * are residual starting material (and respective spinning sidebands). Due to the experimental set up of solid state NMR and the reaction taking place in the rotor reaction rates are considerably slower, and in this case did not go to completion.

¹³C{¹H} solid state NMR (for [1-(ethene)₂][BAr^{F}₄]-Oct; 101 MHz, 10 kHz spin rate) δ: 164.0 (BAr^{F}₄), 134.7 (BArF4), 130.4 (BAr^{F}₄), 125.3 (BAr^{F}₄), 117.2 (BAr^{F}₄), 82.23 (Ethylene) 15-40 (multiple overlapping aliphatic resonances). FIG. 11 shows the solid state ¹³C{¹H} NMR of [1-(ethene)₂][BAr^{F}₄]-Oct, made by the exposure of [1-NBA][BAr^{F}₄] to ethylene (2 bar), in a solid state NMR rotor, for 2 hours. The resonance marked * is a spinning sideband, those marked+are due to a small amount of [1-Butadiene][BAr^{F}₄], which comes from the dehydrogenative coupling of ethylene (vide infra). Due to the experimental set up of solid state NMR and the reaction taking place in the rotor reaction rates are considerably slower.

Mass Spec found (calc.): 581.2189 (581.2907) note: there is considerable presence of [1-butadiene][BAr^{F}₄] and decomposition product of formula m/z=[{(Cy₂PCH₂CH₂PCy₂)Rh}Cl₂]²⁺-H₂. There is no evidence for [1-butadiene][BAr^{F}₄] in bulk samples so it is assumed to form via an in-situ ESI-MS process.

Elemental analysis found (calc.) (carried out with a sample of [1-(ethene)₂][BAr^{F}₄]-Hex): C 51.37% (51.51%), H 4.74% (4.63%). Satisfactory Elemental analysis for [1-(ethene)₂][BAr^{F}₄]-Oct has not been attained due to persistent contamination with excess norbornane.

Crystal structure: The transformation from [1-NBA][BAr^{F}₄] to [1-(ethene)₂][BAr^{F}₄]-Oct is also a singlecrystal to single-crystal one, as shown by an X-ray structure determination at 150 K; and starting from [1-NBD][BAr^{F}₄] this represents a rare example of a sequential reaction sequence for such processes.³⁷ It is believed that the CF₃ groups on the anions results in some plasticity in the solidstate lattice, which allows for the movement of the NBA,³⁸ given that there are no clear channels in the crystal lattice. There is a space group change from to P21/n (Z=4) in [1-NBA][BAr^{F}₄] to C2/c (Z=4) in [1-(ethene)₂][BAr^{F}₄]-Oct on substitution.

FIG. 12 shows the solidstate structure of [1-(ethene)₂][BAr^{F}₄]Oct. (a) Cation showing the numbering scheme, displacement ellipsoids shown at the 50% probability level, 50% disorder component shown as open ellipsoids; (b) Local environment around the cation showing the arrangement of [BAr^{F}₄]⁻ anions; H-atoms are omitted. The final refined structural model has a significant R-factor (10%) which we attribute to an increase in mosaicisity on the singlecrystal tosingle crystal reaction in which the highangle X-ray data is diminished in quality. Nevertheless the refinement is unambiguous and shows a [Rh(Cy₂PCH₂CH₂PCy₂)(η²-C₂H₄)₂]⁺ cation encapsulated by an almost perfect octahedron of [BAr^{F}₄]⁻ anions in the extended lattice. The ethene ligands are disordered over two sites, are canted slightly from lying in the square plane by 14°, and the C═C distance is 1.37(1) Å consistent with a double bond.

In the transformation from [1-(ethene)₂][BAr^{F}₄]-Oct to [1-(ethene)₂][BAr^{F}₄]Hex, the space group change is from monoclinic C2/c (Z=4) to hexagonal P6₃22 (Z=6). FIG. 13a shows the solidstate structure of an isolated cation, which demonstrates that this polymorph has a very similar cation compared with [1-(ethene)₂][BAr^{F}₄]Oct, [e.g. d(C═C)=1.35(1) Å]. The major, unexpected, difference is that the [BAr^{F}₄]⁻ anions now do not form an octahedron around the metal cation, but are arranged so that only 5surround the cation leaving a gap proximate to the {Rh(η²H₂C═CH₂)₂}⁺ fragment (FIG. 13b). This results in ethene ligands that sit in a well defined pocket of [BAr^{F}₄]⁻ anions (FIG. 13c). When inspected down the crystallographic caxis the cations and anions are arranged under 3-fold symmetry so that they form a hexagonal structure of three ion pairs (FIG. 13d), resulting in cylindrical pores that run through the crystalline lattice (FIG. 13e). Moreover, these pores are decorated with the inward pointing {Rh(η²H₂C═CH₂)₂}⁺ fragments, so that the ethene ligands are potentially accessible from the pore channels (FIG. 13f). Taking into account the Van der Waals radii³⁹ this porewidth is just less than 1 nm, and the calculated (PLATON⁴⁰) solventaccessible volume is 25%, making [1-(ethene)₂][BAr^{F}₄]Hex a microporous material.⁴¹ This compares with [1-NBA][BAr^{F}₄] and [1-(ethene)₂][BAr^{F}₄]-Oct in which there are no solventaccessible voids. These pores are presumably filled with solvent, but no definitive regions of electron density that we could assign to pentane (the most likely candidate) or CH₂Cl₂ were found. Thus the calculated solvent accessible volume likely represents the upper limit. The quality of the refinement was reasonable (R=6.6%). There are other, smaller trigonal prismatic, pores but these are formed from the CF₃ groups of the [BAr^{F}₄]⁻ anion and do not contain any {Rh(ethene)₂}⁺ fragments. Crystals of [1-(ethene)₂][BAr^{F}₄]-Hex lose long range order when isolated in bulk by removal of solvent and rapid drying under vacuum, as measured by x-ray crystallography. It is suggested that this is due to loss of the disordered solvent in the pores, as ¹H and ³¹P{1H} solution NMR spectroscopy of this material shows essentially identical signals to [1-(ethene)₂][BAr^{F}₄]-Oct showing that ethene has not been lost; while elemental analysis is consistent with the formulation. Due to this loss in crystallinity, though, it has not been possible to reliably measure solidstate NMR spectra for [1-(ethene)₂][BAr^{F}₄]-Hex.

FIG. 14 shows X-ray powder diffraction patterns for [1-(ethene)₂][BAr^{F}₄]-Oct and for [1-(ethene)₂][BAr^{F}₄]Hex. For [1-(ethene)₂][BAr^{F}₄]-Oct, strong peaks are seen at 2theta=9.1953, 19.1186°. For [1-(ethene)₂][BAr^{F}₄]-Hex, strong peaks are seen at 2theta=3.9514, 6.8133°.

It is noted that the structure of [1-(ethene)₂][BAr^{F}₄]-Oct has an elevated R-factor, as well as a low full θₘₐₓ value. This is primarily due to a loss in high angle data—which is rationalised by the synthetic route (single-crystal to single-crystal to single-crystal!) putting strain on the lattice. For [1-(ethene)₂][BAr^{F}₄]-Hex no such loss of data is presented, however the CheckCif output contains one A alert due to the very large voids in the structure.

1.3—Synthesis and characterisation of [Rh(Cy₂PCH₂CH₂PCy₂)(η²-C₃H₆)][BAr^{F}₄] ([1-propene][BAr^{F}₄])

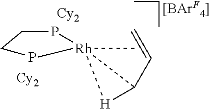

### Attempted Solution Phase Synthesis

A crystalline sample of [1-C₆H₄F₄][BAr^{F}₄] (25 mg, 0.0166 mmol) was taken up in CD₂Cl₂ (0.5 ml) in a high pressure NMR tube. This was freeze-pump-thaw degassed (<1×10⁻² mbar) three times before propylene gas (1 bar) was added. No discernible (by eye) colour change occured. ³¹P{¹H} NMR spectroscopy indicated that very little conversion to [1-propene][BAr^{F}₄] had occurred, with the bulk of the material remaining as the starting [1-C₆H₄F₄][BAr^{F}₄]. Any attempted work-up involving a vacuum results in either starting material or the complete decomposition of the species, to presumed solvent (C—H or C—Cl) activated products (indicated from mass spectroscopy showing the presence of chloride-bridged rhodium dimers). Furthermore leaving [1-propene][BAr^{F}₄] in CH₂Cl₂ solution, at room temperature, resulted in similar decomposition over a period of approximately half an hour. To date it has not been possible to isolate [1-propene][BAr^{F}₄] via solution methods.

### Solid state synthesis of [1-propene][BAr^{F}₄]

To an orange sample of crystalline [1-NBA][BAr^{F}₄] (25 m, 0.0168 mmol) in an evacuated (<1×10⁻² mbar) J Young's flask (c. 50 ml) propylene gas (1 bar, 298 K) is added and left standing overnight. Little colour change is observed, but evidence of a colourless liquid/oil is sometimes observed on the sides of the flask (assumed to be liberated NBA)—it is this sample that was used for spectroscopic analysis. Under a propene atmosphere this compound appears stable for at least 72 hours at room temperature (shown by ³¹P{¹H} solid state NMR). The long-term stability under an argon atmosphere has not been investigated. Attempts to recrystallize the material by dissolving in CH₂Cl₂ led to (presumably solvent induced) decomposition over the period of 30 mins (at room temperature). Dissolving the material in difluorobenzene resulted in the formation of [1-C₆H₄F₂][BAr^{F}₄]. In light of this attempts to recrystallize have been met with failure, and, because of the contamination of norbornane, it has not been possible to attain an acceptable elemental analysis. Yield: Quantitative (>95%) by ³¹P{¹H} solution and solid state NMR (no other signals observed).

### Characterisation of [1-propene](BAr^{F}₄]

¹H solution NMR (CD₂Cl₂, 500 MHz, 298 K) δ: 7.72 (8H, s o-BAr^{F}₄), 7.56 (4H, s, p-BAr^{F}₄), 5.07 (v br, propene), 2.10-1.00 (multiple overlapping aliphatic resonance, i.e. a forest). FIG. 15 shows the solution ¹H NMR spectrum (CD₂Cl₂) of [1-propene][BAr^{F}₄], measured at room temperature, measured immediately upon dissolution. The resonance labelled * is due to residual protio solvent, the labelled+are due to the previously synthesised zwitterionic BAr^{F}₄ complex (1-BAr^{F}₄).

³¹P{¹H} solution NMR (CD₂Cl₂, 202 MHz, 298 K) δ: 95.2 (br d, J_{RhP}=181 Hz). FIG. 16 shows the solution ³¹P{¹H} spectrum of [1-propene][BAr^{F}₄] (Cl₂Cl₂) measured at room temperature (immediately upon dissolution).

¹H solution NMR (CD₂Cl₂, 500 MHz, 193 K) δ: 7.71 (8H, s, o-BAr^{F}₄), 7.54 (4H, s, p-BAr^{F}₄), 4.84 (1H, br, propylene), 4.54 (1H, br, propene), 3.55 (1H, br, propene), 2.02-0.94 (multiple overlapping aliphatic resonances), -0.02 (3H, br, propene agostic CH₃). FIG. 17 shows the solution ¹H NMR spectrum of [1-propene][BAr^{F}₄] measured at 193 K, measured immediately on dissolution and using a pre-cooled spectrometer. The resonance marked * is due to residual protio solvent.

³¹P{¹H} solution NMR (CD₂Cl₂, 202 MHz, 193 K) δ: 100.4 (br, J_{RhP}=200 Hz), 89.9 (br, J_{RhP}=161 Hz). FIG. 18 shows the solution ³¹P{¹H} spectrum of [1-propene][BAr^{F}₄] (CD₂Cl₂) measured at 193 K (immediately upon dissolution).

³¹P{¹H} solid state NMR (162 MHz, 298 K, 10 kHz spin rate) δ: 95.6 (asym. br. s, v_{1/2}=503 Hz). FIG. 19 shows the ³¹P{¹H} solid state NMR of [1-propene][BAr^{F}₄] complex, measured at room temperature. The resonances marked+are due to unknown impurities. The resonances marked * are due to spinning sidebands. The inset is a zoom of the central resonances. The complex is synthesised by direct addition of propylene to [1-NBA][BAr^{F}₄] pre-loaded into the solid state NMR rotor.

³¹P{¹H} solid state NMR (162 MHz, 158 K, 10 kHz spin rate) δ: 101.3 (br, v_{1/2}=510 Hz), 90.4 (br, v_{1/2}=463 Hz). FIG. 20 shows the ³¹P{¹H} solid state NMR of [1-propene][BAr^{F}₄] complex, measured at 158 K. The resonances marked+are due to unknown impurities. The resonances marked * are due to spinning sidebands. The inset is a zoom of the central resonances. The complex is synthesised by direct addition of propylene to [1-NBA][BAr^{F}₄] pre-loaded into the solid state NMR rotor.

FIG. 21 shows a stack plot of the variable temperature solid-state ³¹P{¹H} NMR, demonstrating the coalescence of central resonance.

¹³C{¹H} solid state NMR (101 MHz, 298 K, 10 kHz spin rate) δ: 164.0 (BAr^{F}₄), 134.4 (BAr^{F}₄), 130.4 (BAr^{F}₄), 124.5 (BAr^{F}₄), 118.4 (BAr^{F}₄), 116.9 (BAr^{F}₄), 93.7 (v. br, v_{1/2}=582 Hz), 46-15 (multiple overlapping aliphatic resonances). FIG. 22 shows the ¹³C{¹H} solid state NMR spectrum of [1-propene][BAr^{F}₄], measured at room temperature. The resonance marked * is due to a spinning side band. The inset is a zoom if the broad resonances between 90-100 ppm.

¹³C{¹H} solid state NMR (101 MHz, 158 K, 10 kHz spin rate) δ: 163.7 (BAr^{F}₄), 133.8 (BAr^{F}₄), 130.1 (BAr^{F}₄), 124.6 (BAr^{F}₄), 118.4 (BAr^{F}₄), 116.1 (BAr^{F}₄), 94.2 (Propene C═C), 78.8 (Propene C═C), 46-15 (multiple aliphatic resonances), 6.5 (Propene agostic CH₃). FIG. 23 shows the ¹³C{¹H} solid state NMR spectrum of [1-propene][BAr^{F}₄], measured at 158 K. The resonance marked * is due to a spinning side band. The resonance marked+(˜6 ppm) is the carbon involved in the C—H agostic interaction.

FIG. 24 shows the solid-state fslg-HETCOR 13C/1H spectrum of [1-propene][BAr^{F}₄] the propene complex, measured at 158 K. The cross-peaks assigned to the propene fragment are highlighted.

H/D scrambling in [1-propylene-D₃][BAr^{F}₄]: In an effort to elucidate the precise mechanism of isomerisation of but-1-ene, model experiments were carried out using propylene-D3. [1-NBA][BAr^{F}₄] (20 mg, 0.0135 mmol) was loaded into a high pressure NMR tube in an argon-filled glovebox. This was then sealed using a Teflon stop-cock, before transferring to a Schlenk-line and evacuated. The tube was refilled with propylene-D₃ (1 bar). The head space was then monitored using gas-phase ²H{¹H} NMR.

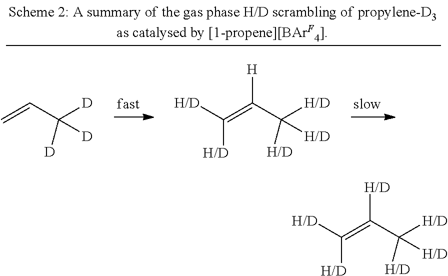

FIG. 25 shows the gas phase ²H{¹H} NMR of the headspace of the reaction of [1-NBA][BAr^{F}₄] with propene-D₃ after 1 hour. The integrals show that scrambling between the end positions (CH₃, 6 -1.7 and CH₂, 6 -5.0) has effectively gone to completion, whereas the central position (CH, 6 -6.0) is still primarily hydrogen. FIG. 26 shows the gas phase ²H{¹H} NMR of the headspace of the reaction of [1-NBA][BAr^{F}₄] with propene-D₃ after 16 hour. The integrals show that scrambling between all positions has effectively gone to completion (CH₃, 6 -1.7; CH₂, 6 -5.0; CH 6 -6.0).

Mass Spec: Not stable under mass spectrometric conditions. Species observed (with appropriate isotopic distributions) at m/z=[{(Cy₂PCH₂CH₂PCy₂)Rh}₂CH₄Cl₂]²⁺; RCy₂PCH₂CH₂PCy₂)Rh(C₄H₈)]⁺;[(Cy₂PCH₂CH₂PCy₂)Rh(C₅H₆)]⁺;[(Cy₂PCH₂CH₂PCy₂)Rh(C₆H₆)]⁺.

Crystal structure: FIG. 27 shows the solid-state structure of [1-propene][BAr^{F}₄]. Displacement ellipsoids are shown at the 30% probability level. (a) Cation with selected hydrogen atoms shown; (b) Disordered propene ligand (with the two components shown in red and white); (c) Packing of the [BAr^{F}₄]⁻ anions with fluorine atoms omitted for clarity.

Similarly to [(1-ethene)₂][BAr^{F}₄]-Oct there is a somewhat elevated R-factor and a low emax value, again due to the loss of high angle data due to crystal quality degrading due to sequential single-crystal to single-crystal transformation.

### 1.4—Synthesis and characterisation of [Rh(Cy₂PCH₂CH₂PCy₂)(η²-C₄H₈)][BAr^{F}₄] ([1-butene][BAr^{F}₄])

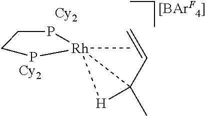

### Attempted Solution Phase Synthesis

A sample of [1-C₆H₄F₂][BAr^{F}₄] (20 mg, 0.0133 mmol) was taken up in CH₂Cl₂ before being freeze-pump-thawed degassed three times and but-1-ene (1 bar) was added. The yellow solution immediately turned orange, and continued to go deeper in colour. It was shown (via ³¹P{¹H} solution NMR spectroscopy), conversion to [1-butadiene][BAr^{F}₄] would occur over the period of one hour in solution.

### Solid state synthesis of [1-butene](BAr^{F}₄]

In order to attain spectroscopic data for [1-butene][BAr^{F}₄] but-1-ene gas (1 bar) is added to an orange sample of crystalline [1-NBA][BAr^{F}₄] (20 mg) in a high pressure NMR tube at room temperature. The solid is allowed to stand for 5 minutes and then is exposed to a dynamic vacuum for 3 minutes (<1×10⁻² mbar). The sample is then dissolved up in CD₂Cl₂ and NMR data immediately recorded. The dehydrogenation to produce the butadiene complex is considerably quicker in solution than in the solid state.

### Characterisation of [1-butene](BAr^{F}₄]

³¹P{¹H} solution NMR (CD₂Cl₂, 202 MHz, 298 K) δ: 95.4 (br. d, J_{RhP}=169 Hz). FIG. 28 shows the solution ³¹P{¹H} NMR spectrum of [1-butene][BAr^{F}₄]. The resonance labelled * is due to the butadiene complex growing in, which begins immediately.

³¹P{¹H} solid state NMR (162 MHz, 298 K, 10 kHz spin rate) δ: 98.4 (br), 95.1 (br). FIG. 29 shows the ³¹P{¹H} solid state NMR spectrum of [1-butene][BAr^{F}₄], 40 minutes after addition. The resonance marked + is due to butadiene complex growing in. The resonances marked * are due to spinning sidebands. The inset is a zoom of the central resonances.

¹³C{¹H} solid state NMR (101 MHz, 298 K, 10 kHz spin rate) δ: 164.3 (BAr^{F}₄), 134.9 (BAr^{F}₄), 130.3 (BAr^{F}₄), 125.1 (BAr^{F}₄), 120.6 (BAr^{F}₄), 118.6 (BAr^{F}₄), 116.7 (BAr^{F}₄), 91.8 (br, butene), 42-15 (multiple overlapping aliphatic resonances), 6.3 (br, butene agostic). FIG. 30 shows the solid state ¹³C{¹H} NMR spectrum of butene complex, 40 minutes after addition.

Mass Spec found (calc.): Under mass spectral conditions the only identifiable signal is due to [1-butadiene][BAr^{F}₄].

Identification of isomer of butene in [1-butene][BAr^{F}₄]: In order to determine which isomer of butane (but-1-ene or but-2-ene) is present in the complex [1-butene][BAr^{F}₄] in the solid state (and thus imply the resting state of the isomerisation catalysis) labelling studies were conducted (Scheme 3). [1-butene][BAr^{F}₄] was made in-situ by addition of but-1-ene (1 bar) to [1-NBA][BAr^{F}₄] (30 mg, 0.0202 mmol) in a high pressure NMR tube. This was allowed to stand for 5 minutes, before subjection to vacuum to remove excess but-1-ene gas (cycled three time), and then D₂ gas was added (1 bar, to form butane-D₂). The deuterated material was dissolved up in CH₂Cl₂ and ²H{¹H} solution NMR was used to identify the locations of the deuterium atoms.

FIG. 31 shows the solution ²H{¹H} NMR of the product of D₂ addition to the in-situ formed [1-butene][BAr^{F}₄] complex. The signal marked * is due to CD₂Cl₂ added for reference.

### 1.5—Synthesis and characterisation of [Rh(Cy₂PCH₂CH₂PCy₂)(η²η²-C₄H₆)][BAr^{F}₄] ([1-butadiene][BAr^{F}₄]) (comparative example)

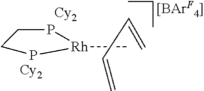

### Synthesis of [1-butadiene](BAr^{F}₄]

To an orange sample of crystalline [1-NBA][BAr^{F}₄] (50 mg, 0.0333 mmol) in a J. Young's flask (c. 100 ml), but-1-ene gas (1 bar) is added and left standing for six hours. Over this time the sample goes a deep burgundy colour. Though crystallinity appears to be retained considerable data loss occurs (for single crystal X-ray diffraction), especially at high angle, and even getting absolute connectivity is not possible. ³¹P{¹H} solution NMR on the dissolved sample showed the product to be formed quantitatively and to be chemically identical to that produced by solution route.

### Characterisation of [1butadiene](BAr^{F}₄]

¹H solution NMR (CD₂Cl₂, 500 MHz) : 7.72 (8H, s, o-BArF4), 7.56 (4H, s, p-BArF4), 5.47 (2H, br t, C2/C3, J_{HH}≈9 Hz), 4.51 (2H, br d, C1/C4, J_{HH}=6 Hz), 2.83 (2H, d, C1/C4, J_{HH}=14 Hz). FIG. 32 shows the solution phase 1H NMR spectrum of [1butadiene][BAr^{F}₄] (CD2Cl2, measured at 298 K). The peak labelled * is residual protio solvent.

³¹P{¹H} solution NMR (CD₂Cl₂, 202 MHz) : 82.0 (d, J_{RhP}=169 Hz). FIG. 33 shows the solution phase 31P{1H} NMR spectrum of [1-butadiene][BAr^{F}₄] measured at 298 K.

³¹P{¹H} solid state NMR δ: 81.0 (asym. br.). FIG. 34 shows the solid state 31P{1H} NMR spectrum of [1-butadiene][BAr^{F}₄] after 6 hours.

¹³C{¹H} solid state NMR δ: 164.3 (BAr^{F}₄), 134.4 (BAr^{F}₄), 130.3 (BAr^{F}₄), 125.1 (BAr^{F}₄), 118.6 (BAr^{F}₄), 116.7 (BAr^{F}₄), 103.5 (butadiene), 99.6 (butadiene), 87.8 (butadiene), 63.2 (butadiene), 42-15 (multiple overlapping aliphatic resonances). FIG. 35 shows the 130{1H} NMR solid state spectrum of [1butadiene][BAr^{F}₄].

Mass Spec found (calc.): 579.2733 (579.2750). Note considerable signal (with appropriate isotopic distribution) at m/z=[(Cy₂PCH₂CH₂PCy₂)Rh(C₂H₄)]⁺; [(Cy₂PCH₂CH₂PCy₂)Rh(C₆H₁₀)]⁺; [(Cy₂PCH₂CH₂PCy₂)Rh(C₇H₁₂)]⁺.

### 1.6—Synthesis and characterization of [Rh(Cy₂P(CH₂)₃PCy₂)(η²:η²-C₈H₁₂)][BAr^{F}₄]

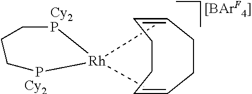

### Synthesis

One Schlenk flask was charged with [Rh(cod)₂][BAr^{F}₄] (350 mg, 0.296 mmol) and dissolved in CH₂Cl₂ (5 mL). Then Cy₂P(CH₂)₃PCy₂ (1.5 mL, 0.2 M solution in C₆H₄F, 0.3 mmol) was added dropwise with vigorous stirring. The resulting light orange solution was allowed to stir for two hours at room temperature before the solvent was partially removed in vacuo (2 mL) and n-pentane (25 mL) was added. The resulting orange solid was filtered via cannula, washed with pentane (3×5 mL), and dried in vacuo to give [Rh(Cy₂P(CH₂)₃PCy₂)(η²:η²-C₈H₁₂)][BAr^{F}₄] as an orange solid. Yield: 400 mg, 0.264 mmol, 89%.

### Characterisation

¹H solution NMR (400.1 MHz, CD₂Cl₂, 298K): δ 7.76 (br s, 8 H, BAr^{F}), 7.60 (s, 4H, BAr^{F}), 5.07 (br s, 4 H, C₈H₁₂), 2.38-2.22 (m, 12H, C₈H₁₂+phosphine), 1.96-1.79 (m, 22H, C₈H₁₂+phosphine), 1.51 (m, 4H, phosphine or C₈H₁₂), 1.42-1.10 (m, 20H, C₈H₁₂+phosphine). ¹¹B{¹H} solution NMR (128.4 MHz, CD₂Cl₂, 298K): δ-6.5 (s). ¹⁹F{¹H} solution NMR (376.5 MHz, CD₂Cl₂, 298K): δ-62.9 (s). ³¹P{¹H} solution NMR (162.0 MHz, CD₂Cl₂, 298K): δ 12.6 (d, J_{RhP2}=139 Hz). Elemental analysis found (calculated) for C₆₇H₇₄P₂F₂₄BRh: C, 53.26 (53.37); H, 4.94 (4.91).

### 1.7—Synthesis and characterization of [Rh(Cy₂P(CH₂)₃PCy₂)(η²:η²-C₇H₈)]]BAr^{F}₄]

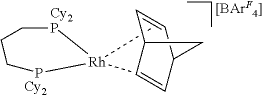

### Synthesis

One Young's flask was charged with [Rh(Cy₂P(CH₂)₃PCy₂)(η²:η²-C₈H₁₂)][BAr^{F}₄] (145 mg, 0.096 mmol) and dissolved in 1,2-F₂C₈H₄ (3 mL). The orange solution was freeze-pump-thaw degassed three times before H₂ gas (1 bar) was added. The reaction mixture was allowed to stir for one hour resulting in lighter orange solution, and then H₂ and solvent were removed in vacuo. The resulting solid was washed with pentane (3×10 mL) and dissolved in CH₂Cl₂ (5 mL). Addition of an excess of norbornadiene (0.14 mL, 1.344 mmol) and stirring for one hour resulted in the darkening of the solution. The solvent was partially removed under vacuum (3.0 mL) and the resulting solution was filtered via cannula into a Young's crystallization tube. Crystals of [Rh(Cy₂P(CH₂)₃PCy₂)(η²:η²-C₇H₈)][BAr^{F}₄] were obtained by layering the resulting solution with n-pentane. Yield: 96 mg, 0.063 mmol, 66%.

### Characterisation

¹H solution NMR (400.1 MHz, CD₂Cl₂, 298K): δ 7.72 (br s, 8H, BAr^{F}), 7.56 (s, 4H, BAr^{F}), 5.16 (br s, 4H, C₇H₈), 4.10 (s, 2 H, C₇H₈), 2.21 (overlapped br s, 2H each, C₇H₈), 1.96-1.73 (m, 26H, phosphine), 1.52 (m, 4H, phosphine), 1.40-1.17 (m, 20H, phosphine). ¹¹B{¹H} solution NMR (128.4 MHz, CD₂Cl₂, 298K): δ-6.5 (s). ¹⁹F{¹H} solution NMR (376.5 MHz, CD₂Cl₂, 298K): δ-62.9 (s). ³¹P{¹H} solution NMR (202.4 MHz, CD₂Cl₂, 298K): δ 15.7 (d, J_{RhP2}=147 Hz). Elemental analysis found (calculated) for C₆₆H₇₀B₁F₂₄F₂Rh₁: C, 53.03 (52.92); H, 4.72 (4.55).

### 1.8—Synthesis and characterization of [Rh(Cy₂P(CH₂)₃PCY₂)(η²:η²-C₇H₁₂)][BAr^{F}₄]

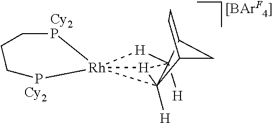

### Synthesis

Addition of H₂ gas (1 bar) to a crystalline samples of [Rh(Cy₂P(CH₂)₃PCY₂)(η²:η²-C₇H₈)][BAr^{F}₄] led to the quantitative formation of [Rh(Cy₂P(CH₂)₃PCy₂)(η²:η²-C₇H₁₂)][BAr^{F}₄] after 5 minutes. The crystalline sample goes opaque and dark upon hydrogenation.

### Characterisation

Single crystal X-ray raw data were collected at 150 K using an Agilent SuperNova diffractometer (Cu Kα radiation, λ=1.54180 Å). Collected crystal lattice parameters: monoclinic (P2/n), a=19.07172(10), b=17.81061(10), c=19.83810(10), β=92.2275(5), V=6733.49(6), Z=4.

### 1.9—Synthesis and characterization of [Rh(Cy₂P(CH₂)₃PCy₂)(η²-propene)][BAr^{F}₄]

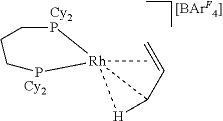

### Synthesis

Addition of propene gas (1 bar) to a crystalline samples of [Rh(Cy₂P(CH₂)₃PCY₂)(η²:η²-C₇H₈)][BAr^{F}₄] led to the quantitative formation of [Rh(Cy₂P(CH₂)₃PCy₂)(η²-Propene)][BAr^{F}₄] after eight hours. The crystalline sample becomes light orange.

### Characterisation

Single crystal X-ray raw data were collected at 100 K using a Rigaku 007 HF (High Flux) diffractometer (Cu Kα radiation, λ=1.54180 Å) equipped with a HyPix-600HE detector. Collected crystal lattice parameters: monoclinic (C2/c), a=19.2343(14), b=16.7377(11), c=20.0147(10), η=91.134(5), V=6442.2(7) Å³, Z=4.

### 1.10—Synthesis and characterization of [Rh(Cy₂P(CH₂)₃PCy₂)(η²:η²-C₈H₁₂)][BAr^{F}₄]

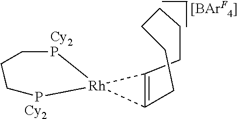

### Synthesis

Addition of H2 gas (1 bar) to a crystalline samples of [Rh(Cy₂P(CH₂)₃PCY₂)(η²:η²-C₈H₁₂)PAr^{F}₄] led to the quantitative formation of RRh(Cy₂P(CH₂)₃PCY₂)(η²:η²-C₈H₁₄)][BAr^{F}₄] after 30 mins. The crystalline sample goes opaque and dark orange upon hydrogenation.

### Characterisation

Single crystal X-ray raw data were collected at 150 K using an Agilent SuperNova diffractometer (Cu Kα radiation, λ=1.54180 Å). Collected crystal lattice parameters: triclinic (P-1), a=13.0186(7), b=13.1664(7), c=20.1179(3), α=87.719(3), β=87.838(3), γ=86.484(4), V=3437.0(3) Å³, Z=2.

### 1.11—Synthesis and characterization of [Rh(Cy₂P(CH₂)₄PCy₂)(η²:η²-C₈H₁₂)][BAr^{F}₄]

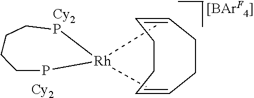

### Synthesis

One Schlenk flask was charged with [Rh(cod)₂][BAr^{F}₄] (270 mg, 0.228 mmol) and another filled with Cy₂P(CH₂)₄PCy (103 mg, 0.228 mmol). Both solids were dissolved in CH₂Cl₂ (3 mL each) and the phosphine was added dropwise to [Rh(cod)₂][BAr^{F}₄] via cannula with vigorous stirring. The resulting light orange solution was allowed to stir for two hours at room temperature before the solvent was partially removed in vacuo (2 mL) and n-pentane (25 mL) was added. The resulting orange solid was filtered via cannula, washed with pentane (3×5 mL), and dried in vacuo to give [Rh(Cy₂P(CH₂)₄PCy₂)(η²:η²-C₈H₁₂)][BAr^{F}₄] as an orange solid. Yield: 300 mg, 0.197 mmol, 86%.

### Characterisation

¹H solution NMR (400.1 MHz, CD₂Cl₂, 298K): δ 7.72 (br s, 8H, BAr^{F}), 7.57 (s, 4H, BAr^{F}), 5.02 (br s, 4 H, C₈H₁₂), 2.37-2.18 (m, 12H, C₈H₁₂+phosphine), 1.86-1.74 (m, 24 H, C₈H₁₂+phosphine), 1.56 (m, 4H, phosphine or C₈H₁₂), 1.37-1.28 (m, 20H, C₈H₁₂+phosphine). ¹¹B{¹H} solution NMR (128.4 MHz, CD₂Cl₂, 298K): δ-6.5 (s). ¹⁹F{¹H} solution NMR (376.5 MHz, CD₂Cl₂, 298K): δ-62.9 (s). ³¹P{¹H} solution NMR (162.0 MHz, CD₂Cl₂, 298K): δ 12.5 (d, J_{RhP2}=140 Hz). Elemental analysis found (calculated) for C₆₈H₇₆B₁F₂₄P₂Rh₁: C, 53.56 (53.49); H, 4.02 (4.91).

### 1.12—Synthesis and characterization of [Rh(Cy₂P(CH₂)₄PCy₂)(η²:η²-C₇H₈)][BAr^{F}₄]

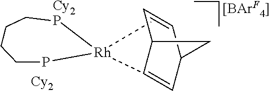

### Synthesis

One Young's flask was charged with Rh(Cy₂P(CH₂)₄PCy₂)(η²:η²-C₈H₁₂)][BAr^{F}₄] (220 mg, 0.144 mmol) and dissolved in 1,2-F₂C₆H₄ (3 mL). The orange solution was freeze-pump-thaw degassed three times before H₂ gas (1 bar) was added. The reaction mixture was allowed to stir for one hour before H₂ and solvent were removed in vacuo. The remaining solid was washed with pentane (3×10 mL) and then dissolved in CH₂Cl₂ (3 mL). Addition of an excess of norbornadiene (0.22 mL, 2.166 mmol) and stirring for one hour resulted in the darkening of the solution. The solvent was partially removed under vacuum (2.0 mL) and the resulting solution was filtered via cannula into a Young's crystallization tube. Crystals of [Rh(Cy₂P(CH₂)₄PCy₂)(η²:η²-C₇H₁₂)][BAr^{F}₄} were obtained by layering the resulting solution with n-pentane. Yield: 168 mg, 0.111 mmol, 77%.

### Characterisation

¹H solution NMR (400.1 MHz, CD₂Cl₂, 298K): δ 7.72 (m, 8 H, BAr^{F}), 7.57 (s, 4H, BAr^{F}), 4.91 (overlapped dt, 4H, J_{HH}=2.5, 1.9 Hz, C₇H₈), 4.04 (br s, 2 H, C₇H₈), 2.15 (br s, 4H, C₇H₈), 1.94-1.68 (m, 30H, phosphine), 1.43-1.21 (m, 22H, phosphine). “B{¹H} solution NMR (128.4 MHz, CD₂Cl₂, 298K): δ-6.6 (s). ¹⁹F{¹H} solution NMR (376.5 MHz, CD₂Cl₂, 298K): 8 -62.9 (s). ³¹P{¹H} solution NMR (162.0 MHz, CD₂Cl₂, 298K): δ 26.8 (d, J_{RhP2}=152 Hz). Elemental analysis found (calculated) for C₆₇H₇₂P₂F₂₄BRh: C, 53.33 (53.26); H, 4.81 (4.60).

### 1.13—Synthesis and characterization of [Rh(Cy₂P(CH₂)₄PCy₂)(η²:η²-C₇H₁₂)]]BAr^{F}₄]

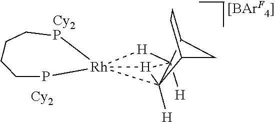

### Synthesis

Addition of H₂ gas (1 bar) to a crystalline samples of [Rh(Cy₂P(CH₂)₄PCY₂)(η²:η²-C₇H₈)][BAr^{F}₄] led to the quantitative formation of [Rh(Cy₂P(CH₂)₄PCy₂)(η²:η²-C₇H₁₂)][BAr^{F}₄] after 5 minutes. The crystalline sample goes opaque and dark upon hydrogenation.

### Characterisation

Single crystal X-ray raw data were collected at 150 K using an Agilent SuperNova diffractometer (Cu Kα radiation, λ=1.54180 Å). Collected crystal lattice parameters: monoclinic (P2/n), a=19.00390(10), b=18.02740(10), c=20.06620(10), β=92.2230(10), V=6869.33(6), Z=4.

### 1.14—Synthesis and characterization of [Rh(Cy₂P(CH₂)₄PCy₂)(η²-propene)][BAr^{F}₄]

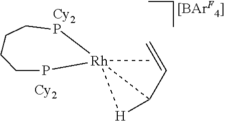

### Synthesis

Addition of propene gas (1 bar) to a crystalline samples of [Rh(Cy₂P(CH₂)₃PCy₂)(η²:η²-C₇H₈)][BAr^{F}₄] led to the quantitative formation of [Rh(Cy₂P(CH₂)₄PCy₂)(η²-Propene)][BAr^{F}₄] after eight hours. The crystalline sample becomes light orange.

### Characterisation

Single crystal X-ray raw data were collected at 100 K using a Rigaku 007 HF (High Flux) diffractometer (Cu Kα radiation, λ=1.54180 Å) equipped with a HyPix-600HE detector. Collected crystal lattice parameters: monoclinic (C2/c), a=18.773(5), b=16.951(2), c=19.809(3), β=90.109(15), V=6303(2) Å³, Z=4.

### 1.15—Synthesis and characterization of Cy₂F(CH₂)₅PCy₂

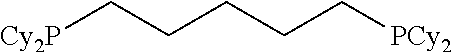

### Synthesis

One Schlenk flask was charged with [Cy₂PLi.(THF)]—(1 g, 3.62 mmol) and suspended in dry 1,4-dioxane (15 mL) at room temperature. Then, 1,5-dibromopentane (0.24 mL, 1.76 mmol) was added dropwise via syringe promptly producing a colourless solution. The solution was stirred at room temperature for two hours yielding a white suspension. The resulting suspension was filtered via cannula and 1,4-dioxane was removed in vacuo to give a colourless solid. This solid was dissolved in dry ethanol (15 mL) upon warming up. Cy₂P(CH₂)₅PCy₂ was obtained as a colorless crystalline solid by storing the resulting solution at 4° C. for 24 h. Yield: 620 mg, 1.33 mmol, 76%.

### Characterisation

¹H solution NMR (400.1 MHz, C₆D₆, 298K): δ 1.89-1.50 (m, 30H), 1.42 (m, 4H, phosphine), 1.32-1.15 (m, 20H). ³¹P{¹H} solution NMR (162.0 MHz, C₆D₆, 298K): δ-5.8 (d, J_{RhP2}=139 Hz). ¹³C{¹H} solution NMR (100.6 MHz, C₆D₆, 298K): 8 34.0 (d, J_{CP}=15 Hz, CH), 30.9 (d, J_{CP}=15 Hz), 29.5 (d, J_{CP}=9 Hz), 28.8 (d, J_{Cp}=21 Hz), 27.76 (d, J_{CP}=17 Hz), 27.74 (br s), 27.0 (s), 21.9 (d, J_{CP}=19 Hz).

### 1.16—Synthesis and characterization of [Rh(Cy₂P(CH₂)₅PCy₂)(η²:η²-C₈H₁₂)][BAr^{F}₄]

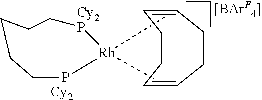

### Synthesis

One Schlenk flask was charged with [Rh(cod)₂][BAr^{F}₄] (500 mg, 0.423 mmol) and another filled with Cy₂P(CH₂)₅PCy (197 mg, 0.423 mmol). Both solids were dissolved in CH₂Cl₂ (5 mL each) and the phosphine was added dropwise to [Rh(cod)₂][BAr^{F}₄] via cannula with vigorous stirring. The resulting light orange solution was allowed to stir for two hours at room temperature before the solvent was partially removed in vacuo (4 mL) and n-pentane (25 mL) was added. The resulting orange solid was filtered via cannula, washed with pentane (3×10 mL), and dried in vacuo to give [Rh(Cy₂P(CH₂)₅PCy₂)(η²:η²-C₈H₁₂)][BAr^{F}₄] as an orange solid.

### Characterisation

¹H solution NMR (400.1 MHz, CD₂Cl₂, 298K): δ 7.72 (br s, 8H, BAr^{F}), 7.56 (s, 4H, BAr^{F}), 4.90 (br s, 4H, C₈H₁₂), 2.37-1.52 (several m, 42H, C₈H₁₂+phosphine), 1.53-1.26 (m, 20H, C₈H₁₂+phosphine). “B{¹H} solution NMR (128.4 MHz, CD₂Cl₂, 298K): δ-6.5 (s). ¹⁹F{¹H} solution NMR (376.5 MHz, CD₂Cl₂, 298K): δ-62.9 (s). ³¹P{¹H} solution NMR (162.0 MHz, CD₂Cl₂, 298K): δ 8.3 (d, J_{RhP2}=138 Hz).

### 1.17—Synthesis and characterization of [Rh(Cy₂P(CH₂)₈PCy₂)(η²:η²-C₇H₈)][BAr^{F}₄]

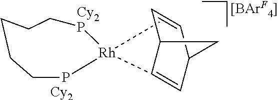

### Synthesis

One Young's flask was charged with [Rh(Cy₂P(CH₂)₈PCy₂)(η²:η²-C₈H₁₂)][BAr^{F}₄] (160 mg, 0.104 mmol) and dissolved in 1,2-F₂C₈H₄ (3 mL). The orange solution was freeze-pump-thaw degassed three times before H₂ gas (1 bar) was added. The reaction mixture was allowed to stir vigorously for 5 mins and it was immediately freeze-pump-thaw degassed three times to remove H₂. n-Pentane (25 mL) was then added to give a pale yellow suspension. The resulting solid was filtered via cannula, washed with pentane (3×10 mL), dried in vacuo and then dissolved in CH₂Cl₂ (3 mL). Addition of an excess of norbornadiene (0.15 mL, 1.47 mmol) and stirring for one hour gave a dark red solution. The solvent was partially removed under vacuum (1.5 mL) and the solution was filtered via cannula into a Young's crystallization tube. Crystals of [Rh(Cy₂P(CH₂)₅PCy₂)(η²:η²-C₇H₈)][BAr^{F}₄] were obtained by layering the resulting solution with n-pentane. Yield: 140 mg, 0.091 mmol, 88%.

### Characterisation

¹H solution NMR (400.1 MHz, CD₂Cl₂, 298K): δ 7.71 (br s, 8H, BAr^{F}), 7.56 (s, 4H, BAr^{F}), 4.69 (br m, 4H, C₇H₈), 3.97 (br s, 2H, C₇H₈), 2.22 (m, 4H, C₇H₈), 1.91-1.68 (m, 34H, phosphine), 1.43-1.21 (m, 20H, phosphine). ¹¹B{¹H} solution NMR (128.4 MHz, CD₂Cl₂, 298K): δ-6.6 (s). ¹⁹F{¹H} solution NMR (376.5 MHz, CD₂Cl₂, 298K): δ-62.9 (s). ³¹P{¹H} solution NMR (162.0 MHz, CD₂Cl₂, 298K): δ 18.7 (d, J_{RhP2}=150 Hz).

### 1.18—Synthesis and characterization of [Rh(Cy₂P(CH₂)₈PCy₂)(η²:η²-C₇H₁₂)][BAr^{F}₄]

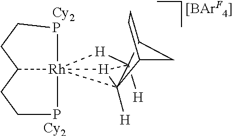

### Synthesis

Addition of H₂ gas (1 bar) to a crystalline samples of [Rh(Cy₂P(CH₂)₃PCy₂)(η²:η²-C₇H₈)][BAr^{F}₄] led to the quantitative formation of a compound of formulae “[Rh(Cy₂P(CH₂)₅PCY₂)(η²:η²-C₇H₁₂)][BAr^{F}₄]” after 5 minutes. The crystalline sample turned yellow upon hydrogenation.

### Characterisation

Single crystal X-ray raw data were collected at 100 K using an Agilent SuperNova diffractometer (Cu Kα radiation, λ=1.54180 Å). Collected crystal lattice parameters: monoclinic (/2/a), a=20.6165(3), b=17.74689(19), c=77.1292(6), β=94.5127(9), V=28132.4(5), Z=20.

### 1.19—Synthesis and characterization of [Rh(Cy₂PCH₂CH₂PCy₂)(NBD)][BAr^{Cl}₄]

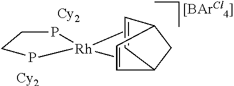

### Synthesis

A stirred slurry of Na[BAr^{Cl}₄] (168 mg, 0.27 mmol) and NBD (0.25 mL) in CH₂Cl₂ (20 mL) was treated with a yellow solution of [Rh(Cy₂PCH₂CH₂PCy₂)Cl]₂ (153 mg, 0.136 mmol) in CH₂Cl₂ (10 mL). The resultant red mixture was stirred at ambient temperature for 4 h and then filtered. The filtrate was concentrated under vacuum (ca. 2 mL) and layered with pentane. Dark orange crystals suitable for an x-ray diffraction study were obtained. Yield: 273 mg (84%).

### Characterisation

¹H NMR (CD₂Cl₂, 400 MHz, 298 K): δ 7.04 (m, 8H, ortho-ArH), 7.01 (t, 4H, para-ArH), 5.53 (br s, 4H, alkene CH), 4.17 (br s, 2H, bridgehead CH), 2.00-1.98 (br d, 4H, overlapping aliphatic CH), 1.93-1.65 (m, 26H, overlapping aliphatic CH), 1.36-1.19 (m, overlapping 16H, aliphatic CH), 1.14-1.04 (m, 4H, overlapping aliphatic CH). ³¹P{¹H} NMR (CD₂Cl₂, 162 MHz): δ 69.9 (d, J_{RhP} 154Hz). ¹¹B{¹H} NMR (CD₂Cl₂, 128 MHz, 298 K): δ-6.9 (s). ³¹P{¹H} SSNMR (162 MHz, 10 kHz spin rate, 294 K): δ 64.7 (d, J_{RhP} 145 Hz), 63.0 (d, J_{RhP} 147 Hz). ¹³C{¹H} SSNMR (101 MHz, 10 kHz spin rate, 294 K):δ 165.3 (br, [BAr^{Cl}₄]⁻), 134.7 ([BAr^{Cl}₄]⁻), 131.1 (br, [BAr^{CL}₄]⁻), 122.5 ([BAr^{Cl}₄]⁻), 88.4 (C═C), 87.4 (C═C), 80.3 (C═C), 79.4 (C═C), 69.8 (bridge C), 55.1 (2C, bridgehead C), 34.2-18.9 (multiple aliphatic resonances). ¹H projection from ¹H/¹³C Frequency Switched Lee-Goldburg HETCOR SSNMR: δ 7.02 (br), 2.18 (br). ESI-MS found (calc.): m/z 617.29 (617.29). Elemental analysis found (calc. for C₅₇H₆₈BCl₈P₂Rh): C 56.31 (56.47), H 5.70 (5.65).

### 1.20—Synthesis and characterization of [Rh(Cy₂PCH₂CH₂PCy₂)(NBD)][BAr(F)₄]

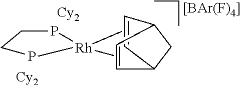

### Synthesis

A stirred slurry of Na[BAr^{F}₄] (91 mg, 0.19 mmol) and NBD (0.25 mL) in CH₂Cl₂ (20 mL) was treated with a yellow solution of [Rh(Cy₂PCH₂CH₂PCy₂)Cl]₂ (96 mg, 0.086 mmol) in CH₂Cl₂ (10 mL). The resultant red mixture was stirred at ambient temperature for 4 h and then filtered. The filtrate was concentrated under vacuum (ca. 1 mL) and layered with pentane. Dark orange crystals suitable for an x-ray diffraction study were obtained. Yield: 154 mg (83%).

### Characterisation

¹H NMR (CD₂Cl₂, 400 MHz, 298 K): δ 6.74 (m, 8H, ortho-ArH), 6.42 (br t, 4H para-ArH), 5.53 (br s, 4H, alkene CH), 4.17 (br s, 2H, bridgehead CH), 2.01-1.98 (br d, 4H, overlapping aliphatic CH), 1.93-1.67 (m, 26H, overlapping aliphatic CH), 1.37-1.19 (m, 16H, overlapping aliphatic CH), 1.14-1.04 (m, 4H, overlapping aliphatic CH). ³¹ P{¹H} NMR (CD₂Cl₂, 162 MHz, 298 K): 6 69.8 (d, J_{RhP} 154 Hz). ¹¹6{¹H} NMR (CD₂Cl₂, 128 MHz, 298 K): δ-6.6 (s). ¹⁹F{¹H} NMR (CD₂Cl₂, 376 MHz, 298 K): δ-115.2 (s). ³¹ P{¹H} SSNMR (162 MHz, 10 kHz spin rate, 294 K): δ 70.9 (br s). ¹³C{¹H} SSNMR (101 MHz, 10 kHz spin rate, 294 K): δ 162.5 (m, [BAr^{F}₄]⁻), 116.8 ([BAr^{F}₄]⁻), 114.3 ([BAr^{F}₄]⁻), 97.7 ([BAr^{F}₄]⁻), 89.3 (C═C), 79.6 (C=C), 71.4 (bridge C), 54.4 (bridgehead C), 34.5-20.7 (multiple aliphatic resonances). ¹H projection from ¹H/¹³C Frequency Switched Lee-Goldburg HETCOR SSNMR: δ 6.42 (br), 2.65 (br). ESI-MS found (calc.): m/z 617.29 (617.29). Elemental analysis found (calc. for C₅₇H₆₈BF₈P₂Rh): C 3.26 (63.34), H 6.42 (6.34).

### 1.21—Synthesis and characterization of [Rh(Cy₂PCH₂CH₂PCy₂)(NBD)][BAr^{H}₄] (comparator)

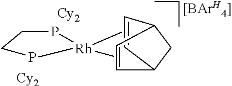

### Synthesis

A stirred slurry of Na[BAr^{H}₄] (55 mg, 0.16 mmol) and NBD (0.25 mL) in CH₂Cl₂ (20 mL) was treated with a yellow solution of [Rh(Cy₂PCH₂CH₂PCy₂)Cl]₂ (90 mg, 0.080 mmol) in CH₂Cl₂ (10 mL). The resultant red mixture was stirred at ambient temperature for 4 h and then filtered. The filtrate was concentrated under vacuum (ca. 1 mL) and layered with pentane. Dark orange crystals suitable for an x-ray diffraction study were obtained. Yield: 117 mg (78%).

### Characterisation

¹H NMR (CD₂Cl₂, 400 MHz, 298 K): δ 7.32 (br m, 8H, ortho-ArH), 7.04 (br t, J_{HH} 7.5 Hz, 8H, meta-ArH), 6.89 (br t, J_{HH} 7.5 Hz, 4H, para-ArH), 5.52 (br s, 4H, alkene CH), 4.16 (s, 2H, bridgehead CH), 1.99 (br d, J_{HH} 12.3 Hz, 4H, overlapping aliphatic CH), 1.92-1.61 (m, 26H, overlapping aliphatic CH), 1.38-1.20 (m, 16H, overlapping aliphatic CH), 1.14-1.01 (m, 4H, overlapping aliphatic CH). ³¹P{¹H} NMR (CD₂Cl₂, 162 MHz, 298 K): δ 69.8 (d, _{JRhP} 154 Hz). ¹¹B{¹H} NMR (CD₂Cl₂, 128 MHz, 298 K): δ-6.6 (s). ³¹ P{¹H} SSNMR (162 MHz, 10 kHz spin rate, 293 K): δ 75.8 (d, J_{RhP} 134 Hz), 64.8 (d, J_{RhP} 132 Hz). ¹³C{¹H} SSNMR (101 MHz, 10 kHz spin rate, 293 K): δ 165.2-158.5 (m, [BAr^{H}₄]⁻), 136.2-135.1 (m, [BAr^{E1}₄]⁻), 125.6-120.7 (m, [BAr^{H}₄]), 89.3 (C═C), 85.4 (C═C), 83.8 (C═C), 81.8 (C═C), 70.6 (bridge C), 54.5 (2C, bridgehead C), 35.8-15.8 (multiple aliphatic resonances). ¹H projection from ¹H/¹³C Frequency Switched Lee-Goldburg HETCOR SSNMR: δ 6.98 (br), 2.00 (br). ESI-MS found (calc.): m/z 617.29 (617.29). Elemental analysis found (calc. for C₅₇H₇₆BP₂Rh): C 73.13 (73.07), H 8.08 (8.18).

### 1.22—Synthesis and characterization of [Rh(Cy₂PCH₂CH₂PCy₂)(NBD)[A{OC(CF₃)₃}₄]

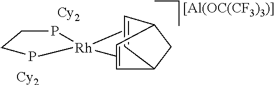

### Synthesis

A solution of [Rh(NBD)₂][Al{OC(CF₃)₃}₄] (123 mg, 0.10 mmol) in CH₂Cl₂ (40 mL) was treated dropwise with a solution of dcpe (42 mg, 0.99 mmol) in CH₂Cl₂ (20 mL) at −60° C. Upon complete addition the color of the reaction solution changed from burgundy to orange. After 2 h, the solution was allowed to warm to ambient temperature. The solvent was then removed under vacuum and the resultant red residue was washed with pentane (3×10 mL). Extraction into CH₂Cl₂ (2 mL) followed by layering with pentane afforded large red crystals suitable for an x-ray diffraction study. Yield: 127 mg (80%).

### Characterisation

¹H NMR (CD₂Cl₂, 400 MHz, 298 K): δ 5.54 (br s, 4H, alkene CH), 4.20 (br s, 2H, bridgehead CH), 2.02-1.98 (br d, 4H, overlapping aliphatic CH), 1.93-1.61 (m, 26H, overlapping aliphatic CH), 1.36-1.21 (m, overlapping 16H, aliphatic CH), 1.14-1.04 (m, 4H, overlapping aliphatic CH). ³¹P{¹H} NMR (CD₂Cl₂, 202 MHz): δ 69.8 (d, J_{RhP} 154Hz). ²⁷Al NMR (CD₂Cl₂, 104 MHz, 298 K): δ 34.6 (s). ³¹P{¹H} SSNMR (162 MHz, 10 kHz spin rate, 294 K): δ 70.2 (d, J_{RhP} 155 Hz), 69.0 (d, J_{RhP} 156 Hz). ¹³C{¹H} SSNMR (101 MHz, 10 kHz spin rate, 294 δ K):121.6 (br q, J_{CF} 280 Hz, CF₃), 94.1 (C═C, 2C), 84.7 (C═C), 82.5 (d, C═C), 79.5 (AIDC), 72.0 (bridge C), 56.5 (bridgehead C), 56.0 (bridgehead C), 38.7-22.3 (multiple aliphatic resonances). ²⁷Al SSNMR (104 MHz, 15 kHz spin rate, 294 K): δ 33.7. ¹H projection from ¹H/¹³C Frequency Switched Lee-Goldburg HETCOR SSNMR: δ 6.00 (br), 4.51 (br), 1.97 (br). ESI-MS found (calc.): m/z 617.29 (617.29). Elemental analysis found (calc. for C₄₃H₅₆AlF₃₆O₄P₂Rh): C 37.08 (37.14), H 3.47 (3.56).

### 1.23—Synthesis and characterization of [Rh(Cy₂PCH₂CH₂PCy₂)(NBA)][BAr^{Cl}₄]

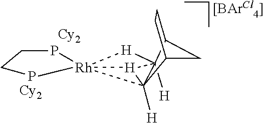

### Synthesis

Hydrogenation (1 atm) of a crystalline sample of [Rh(Cy₂PCH₂CH₂PCy₂)(NBD)][BAr^{Cl}₄] led to the formation of [Rh(Cy₂PCH₂CH₂PCy₂)(NBA)][BAr^{Cl}₄] in 1 h.

### Characterisation

³¹P{¹H} SSNMR (162 MHz, 10 kHz spin rate, 158 K): δ 101.5 (br), 94.7 (br). ¹³C{¹H} SSNMR (101 MHz, 10 kHz spin rate, 158 K):δ 163.7 (br, [BAr^{Cl}₄]⁻), 131.5 (br, [BAr^{Cl}₄]⁻), 121.9 ([BAr^{Cl}₄]⁻), 37.0-14.6 (multiple aliphatic resonances). ¹H projection from ¹H/¹³C Frequency Switched Lee-Goldburg HETCOR SSNMR: δ 7.05 (br), 2.29 (br), −1.76 (br).

### 1.24—Synthesis and characterization of [Rh(Cy₂PCH₂CH₂PCy₂)(NBA)][BAr(F)₄].

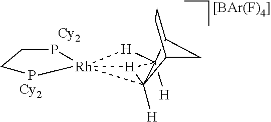

### Synthesis

Hydrogenation (1 atm) of a crystalline sample of [Rh(Cy₂PCH₂CH₂PCy₂)(NBD)][BAr^{F}₄] led to the formation of [Rh(Cy₂PCH₂CH₂PCy₂)(NBA)][BAr^{F}₄] in 3 h.

### Characterisation

³¹P{¹H} SSNMR (162 MHz, 10 kHz spin rate, 158 K): δ 103.6 (br). ¹³C{¹H} SSNMR (101 MHz, 10 kHz spin rate, 158 K):δ 165.5-159.7 (br m, [BAr^{F}₄]⁻), 116.2 ([BAr^{F}₄]⁻), 114.1 ([BAr^{F}₄]⁻), 97.4 ([BAr^{F}₄]⁻), 35.1-20.2 (multiple aliphatic resonances). ¹H projection from ¹H/¹³C Frequency Switched Lee-Goldburg HETCOR SSNMR: δ 6.45 (br), 2.64 (br), −1.62 (br).

### 1.24—Synthesis and characterization of [Rh(Cy₂PCH₂CH₂PCy₂)(H)₂][Al{OC(CF₃)₃}₄]

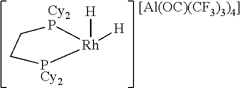

### Synthesis

Hydrogenation (1 atm) of a crystalline sample of [Rh(Cy₂PCH₂CH₂PCy₂)(NBD)] [Al{OC(CF₃)₃}₄] led to the formation of [Rh(Cy₂PCH₂CH₂PCy₂)(H)₂][Al{0C(CF₃)₃}₄] in 1 h.

### Characterisation

³¹P{¹H} SSNMR (162 MHz, 10 kHz spin rate, 294 K): δ 99.5 (br). ¹³C{¹H} SSNMR (101 MHz, 10 kHz spin rate, 294 K):δ 121.6 (br q, J_{CF} 280 Hz, CF₃), 79.3 (AIDC), 38.2-19.9 (multiple aliphatic resonances). ²⁷Al SSNMR (104 MHz, 15 kHz spin rate, 294 K): δ 32.7. ¹H projection from ¹H/¹³C Frequency Switched Lee-Goldburg HETCOR SSNMR: δ 2.00 (br). Elemental analysis found (calc. for C₄₂H₅₀AlF₃₆O₄P₂Rh): C 33.73 (33.75), H 3.19 (3.37).

### Example 2

### Alkene Isomerisation Catalytic Studies

### 2.1—Alkene isomerisation with 1-butene

The complexes [1-NBA][BAr^{F}₄], [1-(ethene)₂][BAr^{F}₄]-Oct, [1-(ethene)₂][BAr^{F}₄]-Hex have been screened (but conditions not optimised) in the isomerization of 1-butene to 2-butene in solid/gas catalysis, acting SMOM-cat. This was performed on a small, but convenient, scale by taking a thick-walled NMR tube of volume ca. 1.9 cm³ fitted with Teflon stopcock that allows for the addition of gases, adding a crystalline sample of catalyst (˜3 mg, ˜2 gmoles), brief evacuation, refilling with 1-butene gas (15 psi, ˜79 gmoles⁷⁸) and analysis by gas-phase ¹H NMR spectroscopy. This loading, assuming all sites in the crystalline material have the same activity, gives TON_{(bulk)} of ˜42 for 100% conversion. This represents a minimum TON, as if only the most accessible sites, or those nearest to the surface, were kinetically competent then the actual number of active sites would be lower. To probe the influence of surface area for [1-NBA][BAr^{F}₄], large (edge length ca. 1-2 mm) crystals and finely crushed samples were prepared for which the surface area would be significantly greater. For both polymorphs of [1-(ethene)₂][BAr^{F}₄] crushed samples were used as large crystals could not be grown (FIG. 36). The samples were not explicitly graded, in the main due to the sensitivity of [1-(ethene)₂][BAr^{F}₄]-Hex, and so the catalytic data presented should be viewed as indicative of the overall rate of isomerization rather than an absolute measure.

### [1-NBA](BAr^{F}₄] (big crystals)

[1-NBA][BAr^{F}₄] (big crystals) was used to catalyse the conversion of but-1-ene to but-2-ene. Conditions used: 2.5 mg catalyst loading, 15 psi but-1-ene, NMR tube volume 1.9 ml. The results are presented in Table 1.

TABLE 1
Data for the catalytic isomerisation of but-1-ene to but-
2-ene by crystals of [1-NBA][BAr₄^{F}] (big crystals).
The conversion was measured by comparing the integrals
of but-1-ene and but-2-ene in the gas phase NMR. The TON
and TOF presented are the minimum possible, assuming all
catalytic sites throughout the bulk to be catalytically active.

| Time (mins) | Time (hr) | % but-2-ene |
|---|---|---|
| 0 | 0.000 | 0 |
| 2 | 0.033 | 55 |
| 5 | 0.083 | 70 |
| 10 | 0.167 | 79 |
| 15 | 0.250 | 84 |
| 20 | 0.333 | 87 |
| 25 | 0.417 | 88 |
| 30 | 0.500 | 88 |
| 35 | 0.583 | 89 |
| 40 | 0.667 | 91 |
| 45 | 0.750 | 91 |
| 50 | 0.833 | 91 |
| 55 | 0.917 | 91 |
| 60 | 1.000 | 93 |
| 100 | 1.667 | 93 |

### [1NBA](BAr^{F}₄] (crushed crystals)

[1NBA][BAr^{F}₄] (crushed crystals) was used to catalyse the conversion of but-1-ene to but-2-ene. Conditions used: 2.0 mg catalyst loading, 15 psi but-1-ene, NMR tube volume 1.9 ml. The results are presented in Table 2.

TABLE 2
Data for the catalytic isomerisation of but-1-ene to but-2-ene by
crystals of [1-NBA][BAr₄^{F}] (crushed crystals). The conversion
was measured by comparing the integrals of but-1-ene and but-2-ene
in the gas phase NMR. The TON and TOF presented are the minimum
possible, assuming all catalytic sites throughout the bulk to be
catalytically active. This dataset is from the initial scoping of
catalysis where the sample was left for 100 mins with no reloading.

| Time (mins) | Time (hr) | % but-2-ene |
|---|---|---|
| 0 | 0.0000 | 0 |
| 1 | 0.0167 | 55 |
| 5 | 0.0833 | 84 |
| 10 | 0.1667 | 92 |
| 15 | 0.2500 | 95 |
| 20 | 0.3333 | 94 |
| 25 | 0.4167 | 95 |
| 30 | 0.5000 | 93 |
| 45 | 0.7500 | 93 |

### [1-(ethene)₂](BAr^{F}₄]-Oct (crushed crystals)

[1-(ethene)₂][BAr^{F}₄]-Oct was used to catalyse the conversion of but-1-ene to but-2-ene. Conditions used: 2.6 mg catalyst loading, 15 psi but-1-ene, NMR tube volume 1.8 ml. The results are presented in Table 3.

TABLE 3
Data for the catalytic isomerisation of but-1-ene to but-
2-ene by [1-(ethene)₂][BAr₄^{F}]-oct. This dataset is
from the initial scoping of catalysis where the sample was
left for 30 mins with no reloading. Conditions used: 2.6
mg catalyst loading, 15 psi but-1-ene, NMR tube volume 1.8 ml.

| Time (mins) | Time (hr) | % but-2-ene |
|---|---|---|
| 0 | 0.000 | 0 |
| 1 | 0.017 | 63 |
| 3 | 0.050 | 79 |
| 5 | 0.083 | 85 |
| 7 | 0.117 | 88 |
| 9 | 0.150 | 89 |
| 10 | 0.167 | 92 |
| 15 | 0.250 | 94 |
| 20 | 0.333 | 95 |
| 25 | 0.417 | 96 |
| 30 | 0.500 | 95 |

### [1-(ethene)₂](BAr^{F}₄]-Hex (crushed crystals)

[1-(ethene)₂][BAr^{F}₄]-Hex was used to catalyse the conversion of but-1-ene to but-2-ene. Conditions used: 6.0 mg catalyst loading, 15 psi but-1-ene, NMR tube volume 1.9 ml. The results are presented in Table 4.

TABLE 4
Data for the catalytic isomerisation of but-1-ene to but-2-ene by [1-
(ethene)₂][BAr₄^{F}]-hex. Due to the high catalytic loading isomerisation
had reached equilibrium by the first data point. Conditions used: 6.0
mg catalyst loading, 15 psi but-1-ene, NMR tube volume 1.9 ml.

| Time (min) | Time (hr) | % but-2-ene |
|---|---|---|
| 0 | 0.000 | 0 |
| 1 | 0.017 | 91 |
| 5 | 0.083 | 91 |
| 10 | 0.167 | 91 |

### Discussion

FIG. 37 shows time/conversion behaviour for the four catalyst systems ([1NBA][BAr^{F}₄] (big crystals), [1-NBA][BAr^{F}₄] (crushed crystals), [1-(ethene)₂][BAr^{F}₄]-Oct (crushed crystals) and [1-(ethene)₂][BAr^{F}₄]-Hex (crushed crystals)), and demonstrates clear structure/activity relationships. FIG. 37 also illustrates the catalytic behaviour of two comparator catalysts containing isobutyl ligands instead of cyclohexyl ligands ([(Bu₂PCH₂CH₂PⁱBu₂)Rh(η²:η²-C₇H₁₂)][BAr^{F}₄] [ⁱBu-NBA] and [(ⁱBu₂PCH₂CH₂P′Bu₂)Rh(η²-η₂H₄)₂][BAr^{F}₄] [ⁱBu-(ethene)₂]).

FIG. 37 illustrates that all four of the exemplary catalysts exhibit superior catalytic activity to the isobutyl-containing comparator catalysts. Of all of the complexes porous [1-(ethene)₂][BAr^{F}₄]-Hex is by far the fastest catalyst, the system reaching equilibrium (˜92% conversion) at the first measured point (1 minute, TOF₍ₘᵢₙ₎=1020 hr⁻¹). Slower, but similar to each other, are [1-(ethene)₂][BAr^{F}₄]-Oct and [1-NBA][BAr^{F}₄]-crushed, reaching completion after 15 minutes (TOF 200-300 hr⁻¹). [1NBA][BAr^{F}₄]large was slower, taking 60 minutes to reach equilibrium (TOF 43 hr⁻¹). All the catalysts yield close to the thermodynamic equilibrium mixture of 1-butene:2-butene of ˜8:92,¹⁴ in a cis:trans ratio of 1:2 as measured by gasphase infrared and ¹H NMR spectroscopy (CD₂Cl₂) of the dissolved gas.

### 2.2. Recyclability Studies

### [1-NBA](BAr^{F}₄] (big crystals)

The recyclability of [1-NBA][BAr^{F}₄] (big crystals) in the conversion of but-1-ene to but-2-ene was assessed. Conditions used: 2.6 mg catalyst loading, 15 psi but-1-ene, 1.8 ml NMR tube volume. The results are presented in Table 5.

TABLE 5
Data for the catalytic isomerisation of but-1-ene to but-2-ene
by a sample of [1-NBA][BAr₄^{F}]-large. This dataset is
from the recyclability experiment - over three reloadings no
significant drop-offs in activity is observed. Conditions used:
2.6 mg catalyst loading, 15 psi but-1-ene, NMR tube volume 1.8 ml.

| | | | Cumulative |
|---|---|---|---|
| Time (mins) | Time (hr) | % but-2-ene | conversion |
| 0 | 0.000 | 0 | 0 |
| 1 | 0.017 | 27 | 27 |
| 3 | 0.050 | 39 | 39 |
| 5 | 0.083 | 47 | 47 |
| 8 | 0.133 | 57 | 57 |
| 10 | 0.167 | 65 | 65 |
| 15 | 0.250 | 71 | 71 |
| 20 | 0.333 | 76 | 76 |
| 25 | 0.417 | 78 | 78 |
| 30 | 0.500 | 80 | 80 |
| 35 | 0.583 | 80 | 80 |
| 40 | 0.667 | 83 | 83 |
| 45 | 0.750 | 84 | 84 |
| 46 | 0.767 | 45 | 129 |
| 48 | 0.800 | 53 | 137 |
| 50 | 0.833 | 61 | 145 |
| 52 | 0.867 | 65 | 149 |
| 55 | 0.917 | 73 | 157 |
| 60 | 1.000 | 78 | 162 |
| 65 | 1.083 | 78 | 162 |
| 70 | 1.167 | 82 | 166 |
| 80 | 1.333 | 87 | 171 |
| 85 | 1.417 | 85 | 169 |
| 90 | 1.500 | 87 | 171 |
| 91 | 1.517 | 29 | 200 |
| 93 | 1.550 | 44 | 215 |
| 95 | 1.583 | 52 | 223 |
| 97 | 1.617 | 62 | 233 |
| 99 | 1.650 | 64 | 235 |
| 100 | 1.667 | 66 | 237 |
| 105 | 1.750 | 73 | 244 |
| 110 | 1.833 | 78 | 249 |
| 115 | 1.917 | 77 | 248 |
| 120 | 2.000 | 78 | 249 |
| 125 | 2.083 | 82 | 253 |
| 130 | 2.167 | 82 | 253 |
| 135 | 2.250 | 83 | 254 |
| 200 | 3.333 | 87 | 258 |

### [1-NBA](BAr^{F}₄] (crushed crystals)

The recyclability of [1-NBA][BAr^{F}₄] (crushed crystals) in the conversion of but-1-ene to but-2-ene was assessed. Conditions used: 3.4 mg catalyst loading, 15 psi but-1-ene, 1.8 ml NMR tube. The results are presented in Table 6.

TABLE 6
Data for the catalytic isomerisation of but-1-ene to
but-2-ene by a sample of [1-NBA][BAr₄^{F}]-crushed.
This dataset is from the recyclability experiment -
over three reloadings no significant drop-offs in activity
is observed. Conditions used: 3.4 mg catalyst loading,
15 psi but-1-ene, NMR tube volume 1.8 ml.

| | | | Cumulative |
|---|---|---|---|
| Time (min) | Time (hr) | % but-2-ene | conversion |
| 0 | 0.000 | 0 | 0 |
| 1 | 0.017 | 20 | 20 |
| 3 | 0.050 | 30 | 30 |
| 5 | 0.083 | 47 | 47 |
| 7 | 0.117 | 66 | 66 |
| 10 | 0.167 | 76 | 76 |
| 15 | 0.250 | 86 | 86 |
| 20 | 0.333 | 88 | 88 |
| 25 | 0.417 | 91 | 91 |
| 30 | 0.500 | 93 | 93 |
| 31 | 0.517 | 12 | 105 |
| 33 | 0.550 | 30 | 123 |
| 35 | 0.583 | 44 | 137 |
| 37 | 0.617 | 56 | 149 |
| 39 | 0.650 | 65 | 158 |
| 40 | 0.667 | 68 | 161 |
| 45 | 0.750 | 78 | 171 |
| 50 | 0.833 | 83 | 176 |
| 55 | 0.917 | 86 | 179 |
| 60 | 1.000 | 90 | 183 |
| 65 | 1.083 | 92 | 185 |
| 70 | 1.167 | 94 | 187 |
| 71 | 1.183 | 15 | 202 |
| 73 | 1.217 | 30 | 217 |
| 75 | 1.250 | 42 | 229 |
| 77 | 1.283 | 55 | 242 |
| 79 | 1.317 | 62 | 249 |
| 80 | 1.333 | 66 | 253 |
| 85 | 1.417 | 71 | 258 |
| 90 | 1.500 | 81 | 268 |
| 95 | 1.583 | 85 | 272 |
| 100 | 1.667 | 87 | 274 |
| 105 | 1.750 | 92 | 279 |
| 110 | 1.833 | 94 | 281 |

### [1-(ethene)₂][BAr^{F}₄]-Oct (crushed crystals)

The recyclability of [1-(ethene)₂][BAr^{F}₄]-Oct (crushed crystals) in the conversion of but-1-ene to but-2-ene was assessed. Conditions used: 2.6 mg cat. Loading, 15 psi but-1-ene, 1.8 ml NMR tube. The results are presented in Table 7.

TABLE 7
Data for the catalytic isomerisation of but-1-ene to but-2-ene
by a sample of [1-(ethene)₂][BAr₄^{F}]-oct. This dataset
is from the recyclability experiment - over three reloadings
no significant drop-offs in activity is observed. Conditions
used: 2.6 mg catalyst loading, 15 psi but-1-ene, NMR tube volume 1.8 ml

| | | | Cumulative |
|---|---|---|---|
| Time (min) | Time (hr) | % but-2-ene | conversion |
| 0 | 0 | 0 | 0 |
| 1 | 0.017 | 63 | 63 |
| 3 | 0.050 | 79 | 79 |
| 5 | 0.083 | 85 | 85 |
| 7 | 0.117 | 88 | 88 |
| 9 | 0.150 | 89 | 89 |
| 10 | 0.167 | 92 | 92 |
| 15 | 0.250 | 94 | 94 |
| 20 | 0.333 | 95 | 95 |
| 25 | 0.417 | 96 | 96 |
| 30 | 0.500 | 95 | 95 |
| 31 | 0.517 | 39 | 134 |
| 33 | 0.550 | 53 | 148 |
| 35 | 0.583 | 62 | 157 |
| 37 | 0.617 | 66 | 161 |
| 39 | 0.650 | 69 | 164 |
| 40 | 0.667 | 72 | 167 |
| 45 | 0.750 | 77 | 172 |
| 50 | 0.833 | 81 | 176 |
| 55 | 0.917 | 86 | 181 |
| 60 | 1.000 | 85 | 180 |
| 65 | 1.083 | 86 | 181 |
| 70 | 1.167 | 89 | 184 |
| 75 | 1.250 | 88 | 183 |
| 76 | 1.267 | 32 | 215 |
| 78 | 1.300 | 45 | 228 |
| 80 | 1.333 | 53 | 236 |
| 82 | 1.367 | 59 | 242 |
| 84 | 1.400 | 64 | 247 |
| 85 | 1.417 | 66 | 249 |
| 90 | 1.500 | 70 | 253 |
| 95 | 1.583 | 75 | 258 |
| 100 | 1.667 | 77 | 260 |
| 105 | 1.750 | 79 | 262 |
| 110 | 1.833 | 83 | 266 |
| 115 | 1.917 | 85 | 268 |
| 120 | 2.000 | 88 | 271 |
| 150 | 2.500 | 97 | 280 |

### [1-(ethene)₂](BAr^{F}₄]-Hex (crushed crystals)

The recyclability of [1(ethene)₂][BAr^{F}₄]-Hex (crushed crystals) in the conversion of but-1-ene to but-2-ene was assessed. Conditions used: 6.0 mg cat. Loading, 15 psi but-1-ene, 1.9 ml NMR tube. The results are presented in Table 7.

TABLE 8
Data for the catalytic isomerisation of but-1-ene to
but-2-ene by a sample of [1-(ethene)₂][BAr₄^{F}]-oct.
This dataset is from the recyclability experiment -
over three reloadings no significant drop-offs in activity
is observed. Conditions used: 6.0 mg catalyst loading,
15 psi but-1-ene, NMR tube volume 1.9 ml.

| | | | Cumulative |
|---|---|---|---|
| Time | Time (hr) | % but-2-ene | conversion |
| 0 | 0.000 | 0 | 0 |
| 1 | 0.017 | 91 | 91 |
| 5 | 0.083 | 91 | 91 |
| 10 | 0.167 | 91 | 91 |
| 15 | 0.250 | 89 | 180 |
| 20 | 0.333 | 91 | 182 |
| 21 | 0.350 | 91 | 182 |
| 22 | 0.367 | 59 | 241 |
| 26 | 0.433 | 89 | 271 |
| 32 | 0.533 | 92 | 274 |

### Discussion

The four catalyst systems ([1-NBA][BAr^{F}₄] (big crystals), [1-NBA][BAr^{F}₄] (crushed crystals), [1-(ethene)₂][BAr^{F}₄]-Oct (crushed crystals) and [1-(ethene)₂][BAr^{F}₄]-Hex (crushed crystals)) can all be recycled, and FIG. 38 shows time/conversion plots for two recharge events, when fresh 1-butene is added immediately after equilibrium has been achieved. All four systems reach the equilibrium position (i.e. ˜92% 2-butene) with a very similar temporal profile compared to the first addition of 1-butene. [1-(ethene)₂][BAr^{F}₄]-Hex shows a drop in activity on recycling which may be due to a partial collapse of the porous network (ToF third charge=500 hr⁻¹), but is still significantly faster than the others. Consistent with this, exposing [1-(ethene)₂][-BAr^{F}₄]-Hex to prolonged dynamic vacuum results in complete loss of activity. For [1-(ethene)₂][BAr^{F}₄]-Oct ten charging cycles have been performed for 1-butane isomerisation, with no appreciable drop in conversion between the first and last recharges (ESI).

In contrast to the exemplary catalysts, comparator catalysts containing isobutyl ligands instead of cyclohexyl ligands ([(Bu₂PCH₂CH₂PⁱBu₂)Rh(η²:η²-C₇H₁₂)][BAr^{F}₄] [ⁱBu-NBA] and [(^{l}Pu₂PCH₂CH₂PBu₂)Rh(η²-C₂H₄)₂HBAr^{F}₄] [ⁱBu-(ethene)₂]) demonstrated no recyclability.

### 2.3—Passivation Studies

Samples of [1-NBA][BAr^{F}₄] (big crystals) and [1-NBA][BAr^{F}₄] (crushed crystals) were exposed to CO (2 bar) for 150 seconds. Solution ³¹P{¹H} NMR showed the sample had gone to 70% completion. [(Cy₂PCH₂CH₂PCy₂)Rh(CO)₂][BAr^{F}₄] is inactive in the catalytic isomerisation of butane. The surface of the crystals (both big and crushed) would react faster than the bulk, effectively turning off the surface for catalysis—the intention being investigating whether this is a surface process or bulk process. However with [1-NBA][BAr^{F}₄] (big crystals) it was noted that significant fracturing of the crystals occurred during the exposure to CO (and presumably the same would be happening on [1-NBA][BAr^{F}₄] (crushed crystals), but not be observable by the naked eye).

Similar studies were carried out with [1-(ethene)₂][BAr^{F}₄]Oct and [1-(ethene)₂][BAr^{F}₄]-Hex, however due to the small amount of sample of both available quantification of the extent of passivation by ³¹ P{¹H} NMR was not possible. The same conditions were used (150 seconds of CO at 2 bar).

### CO-passivated [1-NBA](BAr^{F}₄] (big crystals)

CO-passivated [1-NBA][BAr^{F}₄] (big crystals) was used to catalyse the conversion of but-1-ene to but-2-ene. Conditions used: 2.8 mg cat. Loading, 15 psi but-1-ene, 1.9 ml NMR tube volume. The results are presented in Table 9.

TABLE 9
Data for the catalytic isomerisation of but-1-ene to but-2-ene by
a sample of [1-NBA][BAr₄^{F}]-big after CO passivation

| | | | | min. TOF |
|---|---|---|---|---|
| Time (mins) | Time (hr) | % but-2-ene | min. TON | (hr⁻¹) |
| 0 | 0.000 | 0 | 0.0 | 0.0 |
| 1 | 0.017 | 33 | 13.9 | 831.3 |
| 3 | 0.050 | 47 | 19.7 | 394.7 |
| 5 | 0.083 | 57 | 23.9 | 287.2 |
| 7 | 0.117 | 61 | 25.6 | 219.5 |
| 9 | 0.150 | 66 | 27.7 | 184.7 |
| 10 | 0.167 | 67 | 28.1 | 168.8 |
| 15 | 0.250 | 71 | 29.8 | 119.2 |
| 20 | 0.333 | 74 | 31.1 | 93.2 |
| 25 | 0.417 | 76 | 31.9 | 76.6 |
| 30 | 0.500 | 78 | 32.7 | 65.5 |
| 35 | 0.583 | 79 | 33.2 | 56.9 |
| 40 | 0.667 | 79 | 33.2 | 49.8 |
| 45 | 0.750 | 80 | 33.6 | 44.8 |
| 50 | 0.833 | 81 | 34.0 | 40.8 |
| 55 | 0.917 | 82 | 34.4 | 37.6 |
| 60 | 1.000 | 82 | 34.4 | 34.4 |

FIG. 39 shows time/conversion behaviour for [1-NBA][BAr^{F}₄] (big crystals) and CO-passivated [1-NBA][BAr^{F}₄] (big crystals) in the conversion of 1-butene to 2-butene.

### CO-passivated [1-NBA](BAr^{F}₄] (crushed crystals)

CO-passivated [1-NBA][BAr^{F}₄] (crushed crystals) was used to catalyse the conversion of but-1-ene to but-2-ene. Conditions used: 2.6 mg cat. Loading, 15 psi but-1-ene, 1.8 ml NMR tube volume. The results are presented in Table 10.

TABLE 10
Data for the catalytic isomerisation of but-1-ene to but-2-ene by a
sample of [1-NBA][BAr₄^{F}]-crushed after CO passivation

| | | | | min. TOF |
|---|---|---|---|---|
| Time (mins) | Time (hr) | % but-2-ene | min. TON | (hr⁻¹) |
| 0 | 0.000 | 0 | 0.0 | 0.0 |
| 1 | 0.017 | 23 | 9.9 | 591.9 |
| 3 | 0.050 | 46 | 19.7 | 394.6 |
| 5 | 0.083 | 57 | 24.4 | 293.4 |
| 7 | 0.117 | 65 | 27.9 | 239.0 |
| 9 | 0.150 | 69 | 29.6 | 197.3 |
| 10 | 0.167 | 70 | 30.0 | 180.2 |
| 15 | 0.250 | 73 | 31.3 | 125.3 |
| 20 | 0.333 | 76 | 32.6 | 97.8 |
| 25 | 0.417 | 76 | 32.6 | 78.2 |
| 30 | 0.500 | 78 | 33.5 | 66.9 |
| 35 | 0.583 | 79 | 33.9 | 58.1 |
| 40 | 0.667 | 82 | 35.2 | 52.8 |
| 45 | 0.750 | 81 | 34.7 | 46.3 |

FIG. 40 shows time/conversion behaviour for [1-NBA][BAr^{F}₄] (crushed crystals) and CO-passivated [1-NBA][BAr^{F}₄] (crushed crystals) in the conversion of 1-butene to 2-butene.

### Discussion

It has been shown that addition of CO_{(g)} to crystalline samples of [Rh(Bu₂PCH₂CH₂PBu₂)(η²,η²⁻C₄H₆)][BAr^{F}₄] is slow enough (days) to form a catalytically inactive, passivated, layer of [Rh(Bu₂PCH₂CH₂PBu₂)(CO)₂][BAr^{F}₄] in the resulting crystalline material.⁴² This allows for the activity of surface sites to be probed in catalysis, which were suggested to be considerably more active compared to the bulk. This approach was inspired by the work of Brookhart on single-crystal solid/gas catalysis using [PCP^{iPr}=κ³-C₆H₃-2,6-(OP(C₆H₂-2,4,6-(CF₃)₃)₂]⁴³ For the complexes reported here reaction with CO is much faster, i.e. large crystals of [1-NBA][BAr^{F}₄] react in ˜2 minutes to form [(Cy₂PCH₂CH₂PCy₂)Rh(CO)₂][BAr^{F}₄] in 70% conversion. At the same time considerable cracking of the crystals also occurred, that likely exposes the interior of the crystals.” This means that passivation of just the surface sites is problematic and has therefore not been pursued further with these samples. However, that [1-NBA][BAr^{F}₄]-large shows a significantly lower TOF (based on the bulk) compared to more finely—divided [1-NBA][BAr^{F}₄]-crushed and [1-(ethene)₂][BAr^{F}₄]-Oct suggests that surface effects are import here, and the most active catalyst sites sit at, or near, the surface. This hypothesis is further strengthened by the larger TOF for porous [1-(ethene)₂][BAr^{F}₄]-Hex in which a significant proportion, if not all, of the metal sites are potentially active; pointing as they do into the large cylindrical pores of the single-crystal.

### 2.4. Overview of Catalytic Characteristics

FIGS. 41 and 42 provide an overview of the catalytic properties of the various exemplary catalysts.

In summary, it is believed that catalysts such as [1-(ethene)₂][BAr^{F}₄]-Hex are the first well-defined molecular systems that operate at 298 K under, industrially appealing, solid/gas conditions. In addition, they offer fine control of the spatial environment in the solid-state (i.e. show structure/activity relationships), show TOF₍ₘᵢₙ₎ that are competitive with the fast homogenous systems, and, moreover are recyclable.

### Example 3

### Further Alkene Isomerisation Catalytic Studies

### 3.1—Alkene isomerisation with 1-butene

The ability of compounds [Rh(Cy₂P(CH₂)₃PCy₂)(η²:η²-C₇H₁₂)][BAr^{F}₄], [Rh(Cy₂P(CH₂)₄PCy₂)(η²:η²-C₇H₁₂)][BAr^{F}₄], [Rh(Cy₂P(CH₂)₅PCy₂)(η²:η²-C₇H₁₂)][BAr^{F}₄], [Rh(Cy₂P(CH₂)₃PCy₂)(η²-Propene)][BAr^{F}₄], [Rh(Cy₂P(CH₂)₂PCy₂)(η²:η²-C₇H₁₂)][BAr^{H}₄], [Rh(Cy₂P(CH₂)₂PCy₂)(η²:η²-C₇H₁₂)][BAr(F)₄], [Rh(Cy₂P(CH₂)₂PCy₂)(η²:η²-C₇H₁₂)][BAr^{H}₄] (comparator, prepared in situ) and [Rh(Cy₂P(CH₂)₂PCy₂)(H)₂][Al{OC(CF₃)₃}₄] to catalyse the isomerisation of 1-butene to 2-butene was assessed. The results are presented in Tables 11-18 below:

TABLE 11
Phosphine effects in catalysis. Data for the catalytic
isomerization of but-1-ene to but-2-ene by crystals of
[Rh(Cy₂P(CH₂)₃PCy₂)(η²:η²-C₇H₁₂)][BAr₄^{F}].
The conversion was measured by gas phase ¹H NMR
spectroscopy comparing the integrals corresponding
to 1-butene and 2-butene.

| Time (mins) | Time (hr) | % 2-butene |
|---|---|---|
| 2.35 | 0.039 | 6 |
| 10 | 0.167 | 23 |
| 20 | 0.333 | 44 |
| 30 | 0.500 | 58 |
| 40 | 0.667 | 66 |
| 50 | 0.833 | 71 |
| 60 | 1.00 | 75 |
| 70 | 1.167 | 77 |
| 80 | 1.333 | 79 |
| 90 | 1.500 | 80 |
| 100 | 1.667 | 81 |
| 207 | 3.450 | 90 |

TABLE 12
Phosphine effects in catalysis. Data for the catalytic
isomerization of but-1-ene to but-2-ene by crystals of
[Rh(Cy₂P(CH₂)₄PCy₂)(η²:η²-C₇H₁₂)][BAr₄^{F}].
The conversion was measured by gas phase ¹H NMR spectroscopy
comparing the integrals corresponding to 1-butene and 2-butene

| Time (mins) | Time (hr) | % 2-butene |
|---|---|---|
| 1.65 | 0.028 | 6 |
| 10 | 0.167 | 32 |
| 20 | 0.333 | 45 |
| 30 | 0.500 | 54 |
| 40 | 0.667 | 60 |
| 50 | 0.833 | 65 |
| 60 | 1.00 | 68 |
| 70 | 1.167 | 71 |
| 80 | 1.333 | 73 |
| 90 | 1.500 | 74 |
| 100 | 1.667 | 76 |
| 290 | 4.833 | 90 |

TABLE 13
Phosphine effects in catalysis. Data for the catalytic
isomerization of but-1-ene to but-2-ene by crystals of
[Rh(Cy₂P(CH₂)₅PCy₂)(η²:η²-C₇H₁₂)][BAr₄^{F}].
The conversion was measured by gas phase ¹H NMR spectroscopy
comparing the integrals corresponding to 1-butene and 2-butene.

| Time (mins) | Time (hr) | % 2-butene |
|---|---|---|
| 1.65 | 0.028 | 18 |
| 10 | 0.167 | 53 |
| 20 | 0.333 | 64 |
| 30 | 0.500 | 69 |
| 40 | 0.667 | 73 |
| 50 | 0.833 | 75 |
| 60 | 1.00 | 76 |
| 70 | 1.167 | 78 |
| 80 | 1.333 | 80 |
| 90 | 1.500 | 81 |
| 100 | 1.667 | 82 |
| 205 | 3.417 | 90 |

TABLE 14
Phosphine effects in catalysis. Data for the catalytic
isomerization of but-1-ene to but-2-ene by crystals of
[Rh(Cy₂P(CH₂)₃PCy₂)(η²-propene)][BAr₄^{F}].
The conversion was measured by gas phase ¹H NMR spectroscopy
comparing the integrals corresponding to 1-butene and 2-butene

| Time (mins) | Time (hr) | % 2-butene |
|---|---|---|
| 1.6 | 0.027 | 4 |
| 13.3 | 0.221 | 18 |
| 27.8 | 0.463 | 27 |
| 36.1 | 0.602 | 31 |
| 57.9 | 0.965 | 40 |
| 95.3 | 1.588 | 50 |
| 161.7 | 2.695 | 61 |
| 241.5 | 4.025 | 71 |
| 315.0 | 5.25 | 77 |
| 603.0 | 10.05 | 90 |

TABLE 15
Anion effects in catalysis. Data for the catalytic
isomerization of but-1-ene to but-2-ene by crushed
[Rh(Cy₂P(CH₂)₂PCy₂)(η^{2:}η²-C₇H₁₂)][BAr₄^{Cl}].
The conversion was measured by gas phase ¹H NMR spectroscopy
comparing the integrals corresponding to 1-butene and 2-butene

| Time (mins) | Time (hr) | % 2-butene |
|---|---|---|
| 2.16 | 0.036 | 72 |
| 3.25 | 0.054 | 79 |
| 3.96 | 0.066 | 82 |
| 5.30 | 0.088 | 86 |
| 7.38 | 0.123 | 89 |
| 8.41 | 0.140 | 91 |
| 10.46 | 0.174 | 93 |
| 15.6 | 0.260 | 95 |
| 22 | 0.367 | 96 |

TABLE 16
Anion effects in catalysis. Data for the catalytic
isomerization of but-1-ene to but-2-ene by crushed
[Rh(Cy₂P(CH₂)₂PCy₂)(η²:η²-C₇H₁₂)][BAr(F)₄].
The conversion was measured by gas phase ¹H NMR spectroscopy
comparing the integrals corresponding to 1-butene and 2-butene.

| Time (mins) | Time (hr) | % 2-butene |
|---|---|---|
| 1.6 | 0.028 | 35 |
| 4.6 | 0.077 | 76 |
| 6.7 | 0.113 | 85 |
| 8.2 | 0.137 | 90 |
| 10.3 | 0.172 | 93 |
| 12.5 | 0.208 | 95 |
| 14.9 | 0.250 | 96 |
| 25.5 | 0.425 | 97 |
| 31.8 | 0.530 | 97 |

TABLE 17
Anion effects in catalysis. Data for the catalytic
isomerization of but-1-ene to but-2-ene by crushed
[Rh(Cy₂P(CH₂)₂PCy₂)(η²:η²-C₇H₁₂)][BAr₄^{H}]
(comparator) as prepared in situ. The conversion was measured by gas
phase ¹H NMR spectroscopy comparing the integrals
corresponding to 1-butene and 2-butene

| Time (mins) | Time (hr) | % 2-butene |
|---|---|---|
| 1.25 | 0.021 | 0 |
| 1.96 | 0.033 | 0.5 |
| 2.78 | 0.046 | 1 |
| 3.48 | 0.058 | 1.2 |
| 4.20 | 0.070 | 1.4 |
| 4.93 | 0.082 | 1.5 |
| 5.63 | 0.094 | 1.7 |
| 6.35 | 0.105 | 1.8 |
| 726.35 | 12.21 | 10.5 |

TABLE 18
Anion effects in catalysis. Data for the catalytic
isomerization of but-1-ene to but-2-ene by crushed
[Rh(Cy₂P(CH₂)₂PCy₂)(H)₂][Al{OC(CF₃)₃}₄].
The conversion was measured by gas phase ¹H NMR spectroscopy
comparing the integrals corresponding to 1-butene and 2-butene.

| Time (mins) | Time (hr) | % 2-butene |
|---|---|---|
| 0.92 | 0.015 | 0 |
| 2.40 | 0.040 | 71 |
| 4.18 | 0.070 | 84 |
| 5.65 | 0.094 | 89 |
| 7.33 | 0.122 | 91 |
| 8.70 | 0.145 | 92 |
| 10.16 | 0.169 | 93 |
| 11.70 | 0.195 | 93 |

The data presented in Tables 11-18 show that compounds [Rh(Cy₂P(CH₂)₃PCy₂)(η²:η²-C₇H₁₂)][BAr^{F}₄], [Rh(Cy₂P(CH₂)₄PCy₂)(η²:η²-C₇H₁₂)][BAr^{F}₄], [Rh(Cy₂P(CH₂)₅PCy₂)(η²:η²-C₇H₁₂)][BAr^{F}₄], [Rh(Cy₂P(CH₂)₃PCy₂)(η²-propene)][BAr^{F}₄], [Rh(Cy₂P(CH₂)₂PCy₂)(η²:η²- C₇H₁₂)][BAr^{Cl}₄], [Rh(Cy₂P(CH₂)₂PCy₂)(η²:η²-C₇H₁₂)][BAr(F)₄] and [Rh(Cy₂P(CH₂)₂PCy₂)(H)₂][Al{OC(CF₃)₃}₄] are effective catalysts in the conversion of 1-butene to 2-butene.

### 3.2 Phosphine effects in 1-butene isomerisation

The ability of compounds [Rh(Cy₂P(CH₂)₂PCy₂)(η²:η²-C₇H₁₂)][BAr^{F}₄], [Rh(Cy₂P(CH₂)₅PCy₂)(η²:η²-C₇H₁₂)][BAr^{F}₄], [Rh(Cy₂P(CH₂)₄PCy₂)(η²:η²-C₇H₁₂)][BAr^{F}₄] and [Rh(Cy₂P(CH₂)₃PCy₂)(η²:η²-C₇H₁₂)][BAr^{F}₄] to catalyse the isomerisation of 1-butene to 2-butene was assessed. The results are presented in FIG. 43.

### 3.3 Anion effects in 1-butene isomerisation

The ability of compounds [Rh(Cy₂P(CH₂)₂PCy₂)(η²:η²-C₇H₁₂)][BAr^{F}₄], [Rh(Cy₂P(CH₂)₂PCy₂)(H)₂][Al{OC(CF₃)₃}₄], [Rh(Cy₂P(CH₂)₂PCy₂)(η²:η²-C₇H₁₂)][BAr^{F}₄], [Rh(Cy₂P(CH₂)₂PCy₂)(η²:η²-C₇H₁₂)][BAr(F)₄] and [Rh(Cy₂P(CH₂)₂PCy₂)(η²:η²-C₇H₁₂)][BAr^{F}₄] (comparator, prepared in situ) to catalyse the isomerisation of 1-butene to 2-butene was assessed. The results are presented in FIG. 44.

### 3.4 Scale-up 1-butene isomerisation catalytic studies

Scale-up experiments were performed by loading crystalline samples of each catalyst (1.0-2.5 mg, ca. 0.7-1.7 μmol) into a high-pressure reactor of volume 61 mL fitted with Teflon stopcocks that allows for the addition of 1-butene gas (15-24 psi, 86-138 μmol), see FIG. 45. The isomerization and in situ conversion of 1-butene to 2-butene was monitored and measured by gas-chromatography and gas phase ¹H NMR spectroscopy.

These catalytic loadings gave TON(90%) of ca. 6000 for catalysis. FIG. 46 shows a time vs. conversion plot for [Rh(Cy₂P(CH₂)₂PCy₂)(η²:η²-ethene)₂][BAr^{F}₄]-Hex over 6 repeat cycles.

### Example 4

### Transfer Dehydrogenation Catalytic Studies

The ability for [1-NBA][BAr^{F}₄]-crushed and [1-(ethene)₂][BAr^{F}₄]-Hex to mediate the gas/solid transfer dehydrogenation of butane to butenes has been briefly explored (Scheme 3), as monitored by gas-phase NMR spectroscopy. A typical experiment was as follows a high pressure NMR tube (sealed with a Teflon stopcock) was loaded with 10 mg (0.00673 mmol) of [1-NBA][BAr^{F}₄] in an argon-filled glove box. This was then taken out of the glove box, and evacuated on a Schlenk line (<1×10⁻² mbar). To this butane gas was added (1 bar, 0.0762 mmol)) and the stopcock sealed. The gas feed was changed to ethene and set to the appropriate pressure (Table 19). The glass T-piece and connecting tubing was evacuated and refilled three times (with ethene), before the stopcock was opened. The loaded tubes were left to stand, and the reaction monitored by gas phase ¹H NMR of the head space.

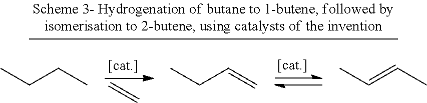

TABLE 19
Butane transfer dehydrogenation data using [1-NBA][
BAr₄^{F}]-crushed and [1-(ethene)₂][BAr₄^{F}]-Hex

| | % | | | | |
|---|---|---|---|---|---|
| | conversion | | | | |
| | butane to | Time | | Ethene:Butane | |
| Catalyst | butene | (hrs) | Temperature | ratio | TON |
| [1-NBA][BAr₄^{F}]-crushed | 63% | 168 | 80 | 2:1 | 3.86 |
| [1-NBA][BAr₄^{F}]-crushed | 40% | 24 | 80 | 2:1 | 2.45 |
| [1-NBA][BAr₄^{F}]-crushed | 15% | 72 | 25 | 1:1 | 1.39 |
| [1-NBA][BAr₄^{F}]-crushed | 27% | 24 | 25 | 1:2 | 3.31 |
| [1-NBA][BAr₄^{F}]-crushed | 33% | 168 | 25 | 1:2 | 4.04 |
| [1-NBA][BAr₄^{F}]-crushed | 18% | 24 | 25 | 1:2 | 2.21 |
| [1-(ethene)₂][BAr₄^{F}]-Hex | 18% | 24 | 25 | 1:2 | 2.21 |

Periodic monitoring of the head space in the NMR tube showed that slow transfer dehydrogenation was occurring to form 2-butene, presumably by slow dehydrogenation to form 1-butene (not observed) and rapid isomerization. For [1-NBA][BAr^{F}₄]-crushed, after 168 hrs at 298 K there was a 33% conversion, which equates to ˜4 turnovers. The catalysis was also shown to operate at 80° C. with an excess of ethene (2:1), under which conditions 68% conversion of butane to butenes is observed (TON=4). Although these turnover numbers are smaller those reported for the best solid-phase molecular catalyst Ir(PCP^{iPr})(C₂H₄) in the pentane/propene system at 240° C. (e.g. TON greater than 1000), the observation of any catalytic activity at 298 K for this challenging reaction is encouraging. It is believed that this is the first time solid/gas transfer dehydrogenation has been reported using a well-defined molecular catalyst at room temperature and low pressures.

### Example 5

### Dimerisation Catalytic Studies

The ability of [1-NBA][BAr^{F}₄]-crushed to effect the dimerization of ethene has been briefly assessed.

FIG. 47 is a gas-phase NMR of [1-NBA][BAr^{F}₄F-crushed left under ethene for two weeks. The resonance marked “+” is due to ethene, whereas the resonances marked “*” are but-2-ene.

While specific embodiments of the invention have been described herein for the purpose of reference and illustration, various modifications will be apparent to a person skilled in the art without departing from the scope of the invention as defined by the appended claims.

### REFERENCES

1. J. F. Hartwig, Organotransition Metal Chemistry, University Science Books, Sausalito, USA, 2010.
2. P. W. N. M. van Leeuwen, Homogeneous Catalysis: Understanding the Art, Springer Netherlands, Dordrecht, 2004.
3. A. Behr and P. Neubert, Applied Homogeneous Catalysis, Wiley VCH, Wienheim, 2012.
4. J. Skupinska, Chem. Rev., 1991, 91, 613-648.
5. J. Mol, Journal of Molecular Catalysis A: Chemical, 2004, 213, 39-45.
6. V. Goelden, D. Linke and E. V. Kondratenko, ACS Catal., 2015, 5, 7437-7445.
7. A. V. Lavrenov, L. F. Saifulina, E. A. Buluchevskii and E. N. Bogdanets, Catal. Ind., 2015, 7, 175-187.
8. M. Taoufik, E. Le Roux, J. Thivolle-Cazat and J. Basset, Angew Chem Int Ed Engl, 2007, 46, 7202-7205.
9. E. Larionov, H. Li and C. Mazet, Chem Commun (Comb), 2014, 50, 9816-9826.
10. R. Cramer and R. V. Lindsey, J. Am. Chem. Soc., 1966, 88, 3534-3544.
11. A. Vasseur, J. Bruffaerts and I. Marek, Nature Chem., 2016, 8, 209-219.
12. C. Chen, T. R. Dugan, W. W. Brennessel, D. J. Weix and P. L. Holland, J. Am. Chem. Soc., 2014, 136, 945-955.
13. H. Kanai, S. B. Choe and K. J. Klabunde, J. Am. Chem. Soc., 1986, 108, 2019-2023.
14. C. A. Tolman, J. Am. Chem. Soc., 1972, 94, 2994-2999.
15. C. S. Higman, L. Plais and D. E. Fogg, ChemCatChem, 2013, 5, 3548-3551.
16. S. Hanessian, S. Giroux and A. Larsson, Organic Letters, 2006, 8, 5481-5484.
17. K. Tanaka, S. Qiao, M. Tobisu, M. M. C. Lo and G. C. Fu, J. Am. Chem. Soc., 2000, 122, 9870-9871.
18. M. Yagupsky and G. Wilkinson, Journal of the Chemical Society A: Inorganic, Physical, Theoretical, 1970, DOI: 10.1039/119700000941, 941-944.
19. S. H. Bergens and B. Bosnich, J. Am. Chem. Soc., 1991, 113, 958-967.
20. S. Biswas, Z. Huang, Y. Choliy, D. Y. Wang, M. Brookhart, K. Krogh-Jespersen and A. S. Goldman, J. Am. Chem. Soc., 2012, 134, 13276-13295.
21. A. R. Chianese, S. E. Shaner, J. A. Tendler, D. M. Pudalov, D. Y. Shopov, D. Kim, S. L. Rogers and A. Mo, Organometallics, 2012, 31, 7359-7367.
22. S. M. M. Knapp, S. E. Shaner, D. Kim, D. Y. Shopov, J. A. Tendler, D. M. Pudalov and A. R. Chianese, Organometallics, 2014, 33, 473-484.
23. T. C. Morrill and C. A. D'Souza, Organometallics, 2003, 22, 1626-1629.
24. M. Mayer, A. Welther and A. Jacobi von Wangelin, ChemCatChem, 2011, 3, 1567-1571.
25. M. Akita, H. Yasuda, K. Nagasuna and A. Nakamura, Bulletin of the Chemical Society of Japan, 1983, 56, 554-558.
26. J. M. Thomas and W. J. Thomas, Principles and practice of heterogeneous catalysis, VCH publishers inc., 3 edn., 2005.
27. C. Copéret, A. Comas-Vives, M. P. Conley, D. P. Estes, A. Fedorov, V. Mougel, H. Nagae, F. NCifiez-Zarur and P. A. Zhizhko, Chem Rev, 2016, 116, 323-421.
28. J. Choi, A. H. R. MacArthur, M. Brookhart and A. S. Goldman, Chem. Rev., 2011, 111, 1761-1779.
29. M. C. Haibach, S. Kundu, M. Brookhart and A. S. Goldman, Acc. Chem. Res., 2012, 45, 947-958.
30. D. C. Leitch, J. A. Labinger and J. E. Bercaw, Organometallics, 2014, 33, 3353-3365.
31. D. C. Leitch, Y. C. Lam, J. A. Labinger and J. E. Bercaw, J. Am. Chem. Soc., 2013, 135, 10302-10305.
32. F. Liu, E. B. Pak, B. Singh, C. M. Jensen and A. S. Goldman, J. Am. Chem. Soc., 1999, 121, 4086-4087.
33. P. J. Perez, Alkane C—H Activation by Single-Site Metal Catalysis, 2012.
34. C. McGlade, J. Speirs and S. Sorrell, Energy, 2013, 55, 571-584.
35. J. Settler, J. Ruiz-Martinez, E. Santillan-Jimenez and B. Weckhuysen, Chem Rev, 2014, 114, 10613-10653.
36. Z. Nawaz, Rev. Chem. Eng., 2015, 31, 413-436.
37. M. E. Van Der Boom, Angew. Chem. Int. Ed., 2011, 50, 11846-11848.
38. S. Libri, M. Mahler, G. Minguez Espallargas, D. C. N. G. Singh, J. Soleimannejad, H. Adams, M. D. Burgard, N. P. Rath, M. Brunelli and L. Brammer, Angew Chem Int Ed Engl, 2008, 47, 1693-1697.
39. S. Alvarez, Dalton Trans, 2013, 42, 8617-8636.
40. A. Spek, J. Appl. Cryst., 2003, 36, 7-13.
41. J. Rouquerol, D. Avnir, C. W. Fairbridge, D. H. Everett, J. H. Haynes, N. Pernicone, J. D. F. Ramsay, K. S. W. Sing and K. K. Unger, Pure and Applied Chemistry, 1994, 66, 1739-1758.
42. S. D. Pike, T. Kra mer, N. H. Rees, S. A. Macgregor and A. S. Weller, Organometallics, 2015, 34, 1487-1497.
43. Z. Huang, P. S. White and M. Brookhart, Nature, 2010, 465, 598-601.
44. M. Olivan, A. V. Marchenko, J. N. Coalter and K. G. Caulton, J. Am. Chem. Soc., 1997, 119, 8389-8390.

1. A catalytic process comprising the step of:
a) activating one or more C—H bonds present within a C₄-C₁₀ hydrocarbon by contacting the C₄-C₁₀ hydrocarbon with a compound having a structure according to formula (I) shown below: 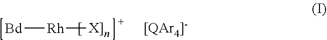
wherein
Bd is a bidentate ligand bonded to Rh via two heteroatoms independently selected from P, N and S,
wherein the two heteroatoms are independently optionally substituted with one or more substituents selected from iso-propyl, tert-butyl, sec-butyl, n-pentyl, iso-pentyl, neo-pentyl, tert-pentyl, sec-pentyl, 3-pentyl, iso-propoxy, tert-butoxy, sec-butoxy, n-pentoxy, iso-pentoxy, neo-pentoxy, tert-pentoxy, sec-pentoxy, 3-pentoxy, 6-8 membered carbocyclyl, 6-8 membered heterocyclyl, aryl or adamantyl, any of which may be optionally substituted with one or more substituents selected from halo, oxo, hydroxyl, (1-4C)alkyl, (2-4C)alkenyl, (2-4C)alkynyl, (1-4C)alkoxy, (1-4C)haloalkyl and —N(Rₓ)(R_{y}),
wherein Rₓ and R_{y} are each independently selected from hydrogen and (1-4C)alkyl;
each X is independently a ligand that is weakly coordinated to Rh via one or more bond;
n is 1, 2 or 3;
Q is selected from B, Al, In and Ga; and
each Ar is independently
i. a phenyl group substituted with one or more substituents selected from halo, (1-3C)alkyl and (1-3C)haloalkyl, or
ii. a (1-3C)alkoxy group substituted with one or more substituents selected from halo, (1-3C)alkyl and (1-3C)haloalkyl.

2. The catalytic process of claim 1, wherein each X is independently a ligand that is weakly coordinated to Rh via one or more bond, wherein the total energy of coordination of Rh to each X is <130 KJ mol⁻¹.

3-5. (canceled)

6. The catalytic process of claim 1, wherein Bd is a bis-phosphine bidentate ligand.

7. The catalytic process of claim 6, wherein the bis-phosphine bidentate ligand has a structure according to formula (II) shown below: 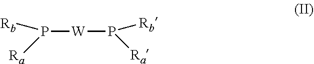
wherein
Rₐ, Rₐ′, R_{E}, and R_{b}′ are each independently iso-propyl, tert-butyl, 6-8 membered carbocyclyl, aryl or adamantyl, any of which may be optionally substituted with one or more substituents selected from halo, hydroxyl, (1-4C)alkyl and (1-4C)alkoxy; and
W is a (1-5C)alkylene linking group optionally substituted with one or more groups R_{c}, wherein each R_{c} is independently (1-4C)alkyl or (1-4C)alkoxy, and/or two groups R_{c} may be linked, such that when taken with the atoms to which they are attached, they form a phenyl group optionally substituted with one or more substituents selected from halo, (1-4C)alkyl and (1-4C)alkoxy.

8. (canceled)

9. The catalytic process of claim 7, wherein Rₐ, Rₐ′, R_{b} and R_{b}′ are cyclohexyl.

10-13. (canceled)

14. The catalytic process of claim 6 wherein the two P atoms are linked by a linking group selected from (1-5C)alkylene, (2-5C)alkenylene and (2-5C)alkynylene, any of which may be optionally substituted with one or more substituents selected from halo, oxo, hydroxyl, (1-4C)alkyl, (2-4C)alkenyl, (2-4C)alkynyl and (1-4C)alkoxy.

15. (canceled)

16. The catalytic process of claim 14, wherein the two P atoms are linked by a methylene, ethylene, propylene, butylene or pentylene linking group.

17-18. (canceled)

19. The catalytic process of claim 1, wherein each X is hydrogen, an alkane, an alkene or dinitrogen.

20-24. (canceled)

25. The catalytic process of claim 1, wherein n is 1 and X is norbornane or n is 2 and each X is ethene.

26. The catalytic process of any preceding claim claim 1, wherein Q is B, Al or In.

27. (canceled)

28. The catalytic process of claim 1, wherein each Ar is either i) a phenyl group substituted at the 3-, 4- and/or 5-position with one or more substituents selected from halo (1-3C)alkyl and (1-3C)haloalkyl, or ii) a (1-3C)alkoxy group substituted with one or more substituents selected from halo (1-3C)alkyl and (1-3C)haloalkyl.

29-35. (canceled)

36. The catalytic process of claim 1, wherein [QAr₄] has any of the following structures: 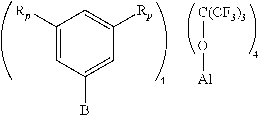
wherein Rₚ is fluoro, chloro or trifluromethyl.

37. The catalytic process of claim 1, wherein the compound according to formula (I) has any one of the following structures 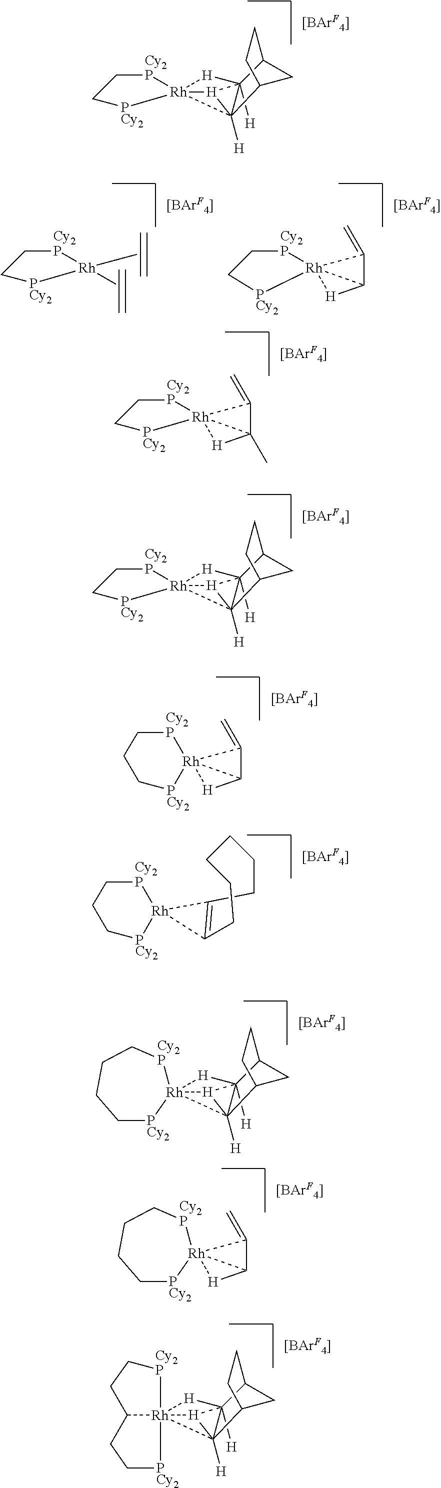 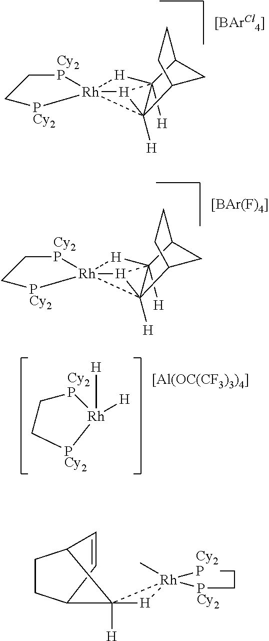
wherein ‘Cy’ denotes cyclohexyl,
‘Ar^{F}’ denotes 3,5-(CF₃)₂C₆H₃,
‘Ar^{Cl}’ denotes 3,5-(Cl)₂C₆H₃, and
‘Ar(F)’ denotes 3,5-(F)₂C₆H₃,

38-42. (canceled)

43. The catalytic process of claim 1, wherein the catalytic process is an alkene isomerisation, alkane transfer dehydrogenation and alkene dimerization.

44. The catalytic process of claim 1, wherein the C₄-C₁₀ hydrocarbon is an alkene comprising one or more C═C bonds, and step a) results in the migration of the one or more C═C bonds within the alkene.

45-48. (canceled)

49. The catalytic process of claim 1, wherein the C₄-C₁₀ hydrocarbon is an alkane, and step a) is conducted in the presence of a hydrogen acceptor, and wherein step a) results in the dehydrogenation of the alkane and the hydrogenation of the hydrogen acceptor.

50-53. (canceled)

54. The catalytic process of claim 1, wherein step a) results in the dimerization of two molecules of the C₄-C₁₀ hydrocarbon.

55. A compound having a structure according to formula (Ia) shown below: 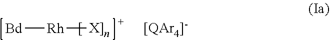
wherein
Bd is a bidentate ligand bonded to Rh via two heteroatoms independently selected from P, N and S,
wherein the two heteroatoms are independently optionally substituted with one or more substituents selected from iso-propyl, tert-butyl, sec-butyl, n-pentyl, iso-pentyl, neo-pentyl, tert-pentyl, sec-pentyl, 3-pentyl, iso-propoxy, iso-butoxy, tert-butoxy, sec-butoxy, n-pentoxy, iso-pentoxy, neo-pentoxy, tert-pentoxy, sec-pentoxy, 3-pentoxy, 6-8 membered carbocyclyl, 6-8 membered heterocyclyl, aryl or adamantyl, any of which may be optionally substituted with one or more substituents selected from halo, oxo, hydroxyl, (1-4C)alkyl, (2-4C)alkenyl, (2-4C)alkynyl, (1-4C)alkoxy, (1-4C)haloalkyl and —N(Rₓ)(R_{y}),
wherein Rₓ and R_{y} are each independently selected from hydrogen and (1-4C)alkyl;
each X is independently a ligand that is weakly bound to Rh via one or more bond, wherein the total energy of coordination of Rh to each X is <130 KJmol⁻¹, and wherein each X is selected from hydrogen, dinitrogen, a linear or branched (2-10C)alkene, a 5-10 membered cycloalkene, a linear or branched (6-10C)alkane and a 8-10 membered cycloalkane, any of which may be optionally substituted with one or more substituents selected from halo, oxo, hydroxyl, (1-4C)alkyl, (2-4C)alkenyl, (2-4C)alkynyl, (1-4C)alkoxy, (1-4C)haloalkyl and —N(Rᵥ)(R_{w}),
wherein Rᵥ and R_{w} are each independently selected from hydrogen and (1-4C)alkyl;
n is 1, 2 or 3;
Q is selected from B, Al, In and Ga; and
each Ar is independently
i. a phenyl group optionally substituted with one or more substituents selected from halo, (1-3C)alkyl and (1-3C)haloalkyl, or
ii. a (1-3C)alkoxy group substituted with one or more substituents selected from halo, (1-3C)alkyl and (1-3C)haloalkyl.

56-58. (canceled)

59. The compound of claim 55, wherein n is 1 and X is selected from propene, butane, pentene, hexadiene and cyclooctene, and/or n is 2 and each X is independently selected from hydrogen, ethene and dinitrogen.

60-63. (canceled)

64. The compound of claim 55, wherein Bd is a bis-phosphine bidentate ligand,
(i) wherein the bis-phosphine bidentate ligand has a structure according to formula (II) shown below: 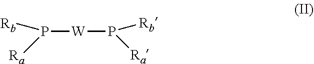
wherein
Rₐ, Rₐ′, R_{b}, and R_{b}′ are each independently iso-propyl, tent-butyl, 6-8 membered carbocyclyl, aryl or adamantyl, any of which may be optionally substituted with one or more substituents selected from halo, hydroxyl, (1-4C)alkyl and (1-4C)alkoxy; and
W is a (1-5C)alkylene linking group optionally substituted with one or more groups R_{c}, wherein each R_{c} is independently (1-4C)alkyl or (1-4C)alkoxy, and/or two groups R_{c} may be linked, such that when taken with the atoms to which they are attached, they form a phenyl group optionally substituted with one or more substituents selected from halo, (1-4C)alkyl and (1-4C)alkoxy, and/or
(ii) wherein the two P atoms in the bis-phosphine bidentate ligand are linked by a linking group selected from (1-5C)alkylene, (2-5C)alkenylene and (2-5C)alkynylene, any of which may be optionally substituted with one or more substituents selected from halo, oxo, hydroxyl, (1-4C)alkyl, (2-4C)alkenyl, (2-4C)alkynyl and (1-4C)alkoxy.

65. The compound of claim 55, wherein Q is B, Al or In.

66. The compound of claim 55, wherein
Ar is either i) a phenyl group substituted at the 3-, 4- and/or 5-position with one or more substituents selected from halo (1-3C)alkyl and (1-3C)haloalkyl, or ii) a (1-3C)alkoxy group substituted with one or more substituents selected from halo (1-3C)alkyl and (1-3C)haloalkyl, and/or
[QAr₄] has any of the following structures: 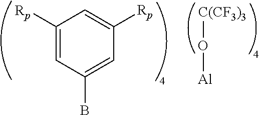
wherein Rₚ is fluoro, chloro or trifluromethyl.

67. (canceled)

68. The compound of claim 55,
wherein the compound has any one of the following structures 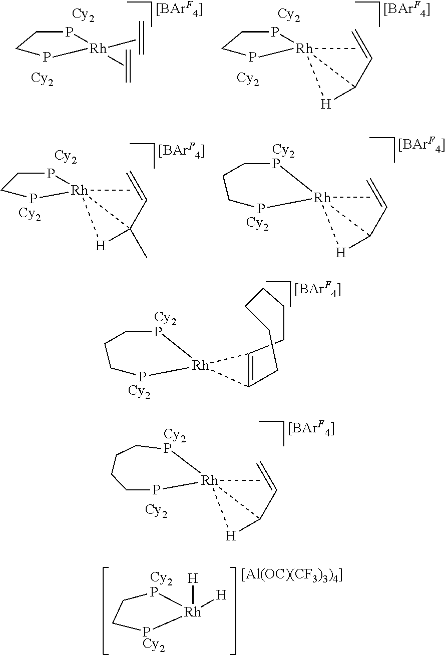
wherein ‘Cy’ denotes cyclohexyl and ‘Ar^{F}’ denotes 3,5-(CF₃)₂C₆H₃.

69-73.(canceled)
